(19) United States
(12) Patent Application Publication
(10) Pub. No.: US 2026/0120879 A1
(43) Pub. Date: Apr. 30, 2026

(54) MACHINE LEARNING TECHNIQUE FOR IDENTIFYING ICI RESPONDERS AND NON-RESPONDERS

(71) Applicant: BostonGene Corporation, Waltham, MA (US)

(72) Inventors: Aleksandr Zaitsev, Yerevan (AM); Anastasiia Nikitina, Waltham, MA (US); Michael F. Goldberg, Brookline, MA (US); Evgenii Bolshakov, Yerevan (AM); Ravshan Ataullakhanov, Moscow (RU); Tatiana Vasileva, Yerevan (AM); Anastasiia Terenteva, Yerevan (AM)

(73) Assignee: BostonGene Corporation, Waltham, MA (US)

(21) Appl. No.: 18/933,893

(22) Filed: Oct. 31, 2024

Related U.S. Application Data
(60) Provisional application No. 63/594,948, filed on Oct. 31, 2023.

(51) Int. Cl. G16H 50/30; G16B 25/10; G16H 20/10

(52) U.S. Cl. CPC G16H 50/30; G16B 25/10; G16H 20/10

(57) ABSTRACT

Described herein are techniques for predicting whether a subject will respond to an immune checkpoint inhibitor (ICI) therapy based on RNA expression data and cytometry data obtained for the subject. In some embodiments, the techniques include: obtaining the RNA expression data, the RNA expression data having been previously obtained from a tumor sample from the subject; selecting, using the RNA expression data, an MF profile type for the tumor sample; obtaining the cytometry data, the cytometry data having been previously obtained from a blood sample from the subject; determining, using the cytometry data, a G2 score for the blood sample, wherein the G2 score is indicative a likelihood that the blood sample is of a Primed (G2) immunoprofile type of multiple immunoprofile types; and predicting, based on the selected MF profile type and the G2 score, whether the subject will respond to the ICI therapy.

### CROSS-REFERENCE TO RELATED APPLICATIONS

This application claims the benefit under 35 U.S.C. § 119 (e) of the filing date of U.S. Provisional Application No. 63/594,948, filed Oct. 31, 2023, and entitled “MACHINE LEARNING TECHNIQUE FOR IDENTIFYING ICI RESPONDERS AND NON-RESPONDERS,” the entire contents of which are incorporated by reference herein.

### BACKGROUND

In general, a tumor mass (or other diseased tissue) may comprise a population of malignant cells (e.g., cancer cells) and a microenvironment which may include, for example, immune cells, surrounding blood vessels, and fibroblasts.

The immune system is a complex network of biological systems that protects an organism against diseases, including cancer. The immune system includes white blood cells, which circulate in the blood and lymphatic vessels.

### SUMMARY

Some aspects provide for a method for predicting whether a subject will respond to an immune checkpoint inhibitor (ICI) therapy based on RNA expression data and cytometry data obtained for the subject, the method comprising: using at least one computer hardware processor to perform: obtaining the RNA expression data, the RNA expression data having been previously obtained from a tumor sample from the subject; selecting, from among multiple molecular-functional (MF) profile types and using the RNA expression data, an MF profile type for the tumor sample; obtaining the cytometry data, the cytometry data having been previously obtained from a blood sample from the subject; determining, using the cytometry data, a G2 score for the blood sample, wherein the G2 score is indicative a likelihood that the blood sample is of a Primed (G2) immunoprofile type of multiple immunoprofile types; and predicting, using a statistical model and based on the selected MF profile type and the G2 score, whether the subject will respond to the ICI therapy.

Some aspects provide for a system comprising: at least one computer hardware processor; and at least one non-transitory computer-readable storage medium storing processor-executable instructions that, when executed by the at least one computer hardware processor to perform a method for predicting whether a subject will respond to an immune checkpoint inhibitor (ICI) therapy based on RNA expression data and cytometry data obtained for the subject, the method comprising: obtaining the RNA expression data, the RNA expression data having been previously obtained from a tumor sample from the subject; selecting, from among multiple molecular-functional (MF) profile types and using the RNA expression data, an MF profile type for the tumor sample; obtaining the cytometry data, the cytometry data having been previously obtained from a blood sample from the subject; determining, using the cytometry data, a G2 score for the blood sample, wherein the G2 score is indicative a likelihood that the blood sample is of a Primed (G2) immunoprofile type of multiple immunoprofile types; and predicting, using a statistical model and based on the selected MF profile type and the G2 score, whether the subject will respond to the ICI therapy.

Some aspects provide for at least one non-transitory computer-readable storage medium storing processor-executable instructions that, when executed by at least one computer hardware processor, cause the at least one computer hardware processor to perform a method for predicting whether a subject will respond to an immune checkpoint inhibitor (ICI) therapy based on RNA expression data and cytometry data obtained for the subject, the method comprising: obtaining the RNA expression data, the RNA expression data having been previously obtained from a tumor sample from the subject; selecting, from among multiple molecular-functional (MF) profile types and using the RNA expression data, an MF profile type for the tumor sample; obtaining the cytometry data, the cytometry data having been previously obtained from a blood sample from the subject; determining, using the cytometry data, a G2 score for the blood sample, wherein the G2 score is indicative a likelihood that the blood sample is of a Primed (G2) immunoprofile type of multiple immunoprofile types; and predicting, using a statistical model and based on the selected MF profile type and the G2 score, whether the subject will respond to the ICI therapy.

Some aspects provide for method for predicting whether a subject will respond to an immune checkpoint inhibitor (ICI) therapy based on RNA expression data and cell population data obtained for the subject, the method comprising: using at least one computer hardware processor to perform: obtaining the RNA expression data, the RNA expression data having been previously obtained from a tumor sample from the subject; selecting, from among multiple molecular-functional (MF) profile types and using the RNA expression data, an MF profile type for the tumor sample; obtaining the cell population data, the cell population data having been previously obtained from a blood sample from the subject; determining, using the cell population data, a G2 score for the blood sample, wherein the G2 score is indicative a likelihood that the blood sample is of a Primed (G2) immunoprofile type of multiple immunoprofile types; and predicting, using a statistical model and based on the selected MF profile type and the G2 score, whether the subject will respond to the ICI therapy.

Some aspects provide for a system comprising: at least one computer hardware processor; and at least one non-transitory computer-readable storage medium storing processor-executable instructions that, when executed by the at least one computer hardware processor to perform a method for predicting whether a subject will respond to an immune checkpoint inhibitor (ICI) therapy based on RNA expression data and cell population data obtained for the subject, the method comprising: obtaining the RNA expression data, the RNA expression data having been previously obtained from a tumor sample from the subject; selecting, from among multiple molecular-functional (MF) profile types and using the RNA expression data, an MF profile type for the tumor sample; obtaining the cell population data, the cell population data having been previously obtained from a blood sample from the subject; determining, using the cell population data, a G2 score for the blood sample, wherein the G2 score is indicative a likelihood that the blood sample is of a Primed (G2) immunoprofile type of multiple immunoprofile types; and predicting, using a statistical model and based on the selected MF profile type and the G2 score, whether the subject will respond to the ICI therapy.

Some aspects provide for at least one non-transitory computer-readable storage medium storing processor-executable instructions that, when executed by at least one computer hardware processor, cause the at least one computer hardware processor to perform a method for predicting whether a subject will respond to an immune checkpoint inhibitor (ICI) therapy based on RNA expression data and cell population data obtained for the subject, the method comprising: obtaining the RNA expression data, the RNA expression data having been previously obtained from a tumor sample from the subject; selecting, from among multiple molecular-functional (MF) profile types and using the RNA expression data, an MF profile type for the tumor sample; obtaining the cell population data, the cell population data having been previously obtained from a blood sample from the subject; determining, using the cell population data, a G2 score for the blood sample, wherein the G2 score is indicative a likelihood that the blood sample is of a Primed (G2) immunoprofile type of multiple immunoprofile types; and predicting, using a statistical model and based on the selected MF profile type and the G2 score, whether the subject will respond to the ICI therapy.

Some aspects provide for a method for predicting whether a subject will respond to an immune checkpoint inhibitor (ICI) therapy, the method comprising: using at least one computer hardware processor to perform: obtaining RNA expression data, the RNA expression data having been previously obtained from a tumor sample from the subject; selecting, from among multiple molecular-functional (MF) profile types and using the RNA expression data, an MF profile type for the tumor sample; obtaining a G2 score, wherein the G2 score is a score obtained using a G2 statistical model trained to predict a likelihood that the blood sample is of a Primed (G2) immunoprofile type using as input a plurality of cell composition percentages for a respective plurality of cell types in the blood sample; and determining whether the subject will respond to the ICI therapy using a statistical trained to predict a likelihood that the subject will respond to the ICI therapy using as input the G2 score and the selected MF profile type.

Embodiments of any of the above aspects may have one or more of the following features.

Some embodiments further comprise: after predicting that the subject will respond to the ICI therapy, recommending the ICI therapy for the subject or selecting the subject for treatment with the ICI therapy.

Some embodiments further comprise: administering the ICI therapy to the subject.

Some embodiments further comprise: a method of treating a subject who has been diagnosed as having a tumor, the method comprising: predicting whether the subject will respond to the ICI therapy using a method as described herein, and administering the ICI therapy to the subject when the subject has been determined as likely to respond to the ICI therapy.

In some embodiments, the ICI therapy comprises anti-PD-1 antibodies, anti-CTLA4 antibodies, and/or anti-PD-L1 antibodies.

In some embodiments, predicting whether the subject will respond to the ICI therapy comprises processing the selected MF profile type and the G2 score with the statistical model.

In some embodiments, the statistical model is a generalized linear model.

In some embodiments, the generalized linear model is a logistic regression model.

Some embodiments further comprise: determining, based on the RNA expression data, an expression of PD-L1 in the tumor sample, wherein determining whether the subject will respond to the ICI therapy comprises processing the selected MF profile type, the G2 score, and the expression of PD-L1 in the tumor sample using the statistical model.

In some embodiments, selecting the MF profile type for the tumor sample comprises: determining, using the RNA expression data, an MF profile for the tumor sample at least in part by determining a gene group expression level for each gene group in a set of gene groups; and selecting, using the MF profile, the MF profile type for the tumor sample.

Some embodiments further comprise: encoding the MF profile type selected for the tumor sample to obtain an encoded MF profile type, the encoding comprising: assigning a first value to the MF profile type when the MF profile type is a first MF profile type or a second MF profile type of the multiple MF profile types; and assigning a second value to the MF profile type when the MF profile type is a third MF profile type or a fourth MF profile type of the multiple MF profile types, wherein the second value is different from the first value.

In some embodiments, determining whether the subject will respond to the ICI therapy based on the selected MF profile type and the G2 score comprises: determining whether the subject will respond to the ICI therapy based on the encoded MF profile type and the G2 score.

In some embodiments, the first MF profile type is associated with inflamed and vascularized tumor samples and/or inflamed and fibroblast-enriched tumor samples, the second MF profile type is associated with inflamed and non-vascularized tumor samples and/or inflamed and non-fibroblast-enriched tumor samples, the third MF profile type is associated with non-inflamed and vascularized tumor samples and/or non-inflamed and fibroblast-enriched tumor samples, and the fourth MF profile type is associated with non-inflamed and non-vascularized tumor samples and/or non-inflamed and non-fibroblast-enriched tumor samples,

Some embodiments further comprise: obtaining the tumor sample from the subject.

Some embodiments further comprise: performing RNA sequencing of the tumor sample to obtain the RNA expression data.

In some embodiments, determining the G2 score using the cytometry data comprises: processing the cytometry data to determine cytometry-based cell composition percentages for a plurality of types of cells in the blood sample; and determining the G2 score using the cytometry-based cell composition percentages.

In some embodiments, determining the G2 score using the cytometry-based cell composition percentages comprises processing the cytometry-based cell composition percentages using a G2 score statistical model trained to predict the G2 score.

In some embodiments, processing the cytometry data to determine the cytometry-based cell composition percentages comprises: processing the cytometry data using one or more machine learning models to identify the types of the cells in the blood sample; and determining the cytometry-based cell composition percentages based on the identified types of the cells in the blood sample.

In some embodiments, the RNA expression data for the tumor sample is first RNA expression data. Some embodiments further comprise: obtaining second RNA expression data, the second RNA expression data having been previously obtained from the blood sample from the subject. In some embodiments, determining the G2 score comprises determining the G2 score using the cytometry data or the second RNA expression data.

In some embodiments, determining the G2 score using the second RNA expression data comprises: processing the second RNA expression data to determine RNA-based cell composition percentages for types of cells in the blood sample; and determining the G2 score using the RNA-based cell composition percentages.

In some embodiments, determining the G2 score using the RNA-based cell composition percentages comprises: processing the RNA-based cell composition percentages using a G2 score statistical model trained to predict the G2 score.

In some embodiments, processing the second RNA expression data to determine the RNA-based cell composition percentages comprises: processing the second RNA expression data using non-linear regression models corresponding respectively to the types of cells to obtain the RNA-based cell composition percentages.

Some embodiments further comprise performing RNA sequencing of the blood sample to obtain the second RNA expression data for the blood sample.

Some embodiments further comprise: obtaining the blood sample from the subject.

In some embodiments, the cytometry data is flow cytometry data.

Some embodiments further comprise: processing the blood sample using a cytometry platform to obtain the cytometry data.

In some embodiments, the multiple immunoprofile types comprise: a Naive (G1) immunoprofile type, the Primed (G2) immunoprofile type, a Progressive (G3) immunoprofile type, a Chronic (G4) immunoprofile type, and a Suppressive (G5) immunoprofile type.

In some embodiments, the subject has, is suspected of having, or is at risk of having carcinoma.

In some embodiments, the carcinoma is head and neck squamous cell carcinoma (HNSCC).

In some embodiments: determining the G2 score using the cell population data comprises: processing the cell population data to determine cell composition percentages for types of cells in the blood sample; and determining the G2 score using the cell composition percentages.

In some embodiments, determining the G2 score using the cell composition percentages comprises processing the cell composition percentages using a G2 score statistical model trained to predict the G2 score.

In some embodiments, the cell population data comprises blood RNA expression data or cytometry data, and wherein processing the cell population data to determine the cell composition percentages comprises: processing the blood RNA expression data or cytometry data using one or more machine learning models to identify the types of the cells in the blood sample; and determining the cell composition percentages based on the identified types of the cells in the blood sample.

In some embodiments, the cell population data is cytometry data, sequencing data, hematology data, or multiplex immunofluorescence (MIxF) data.

In some embodiments, the cell population data comprises the cytometry data, and the cytometry data comprises flow cytometry data, mass cytometry data, or spectral cytometry data.

In some embodiments, the cell population data comprises the sequencing data, and the sequencing data comprises bulk RNA sequencing (RNA-seq) data, single cell RNA-seq data, cellular indexing of transcriptomes and epitopes by sequencing (CITE-seq) data, or DNA methylation data.

Some embodiments further comprise: processing the blood sample using an immune platform and/or a sequencing platform to obtain the cell population data.

In some embodiments, the immune platform is a flow cytometry platform, a mass cytometry platform, a spectral cytometry platform, a hematology analyzer, a sequencing platform, or a MIxF imaging platform.

In some embodiments, selecting the MF profile type for the tumor sample comprises: determining, using the RNA expression data, an MF profile for the tumor sample, wherein the MF profile comprises a plurality of gene expression levels and/or gene group expression levels for a respective plurality of predetermined genes and/or gene groups; and selecting the MF profile type for the tumor sample by identifying a cluster of MF profiles from among a set of clusters of MF profiles that the MF profile is associated with, each cluster being associated with a respective MF profile type.

In some embodiments, the MF profiles included in the set of clusters are training MF profiles from a plurality of subjects.

In some embodiments, the MF profile types comprise: a first MF profile type characterized as immune-enriched and fibrotic, a second MF profile type characterized as immune-enriched and non-fibrotic, a third MF profile type characterized as fibrotic and non-immune-enriched, and a fourth MF profile type characterized as immune desert.

Some embodiments further comprise: obtaining flow cytometry data, mass cytometry data, spectral cytometry data, hematology data, sequencing data, and/or imaging data; and determining the plurality of cell composition percentages using the flow cytometry data, mass cytometry data, spectral cytometry data, hematology data, sequencing data, and/or imaging data.

In some embodiments, the plurality of cell types are immune cells.

In some embodiments, the plurality of cell types are the cell types listed in Table 2.

In some embodiments, the plurality of cell types are the cell types listed in Table 3.

In some embodiments, the plurality of types of cells are the cell types listed in Table 4.

In some embodiments, the G2 score statistical model is a machine learning model that has been trained using training data comprising cell composition percentages for a plurality of blood samples associated with the Primed (G2) immunoprofile type and cell composition percentages for a plurality of blood samples associated with one or more immunoprofile types other than the Primed (G2) immunoprofile type.

In some embodiments, the statistical model has been trained using training data comprising G2 scores and MF profile types for a first plurality of training samples from ICI responders and a second plurality of training samples from ICI non-responders.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A and FIG. 1B are diagrams of illustrative techniques for predicting whether a subject will respond to an immune checkpoint inhibitor (ICI) therapy, according to some embodiments of the technology described herein.

FIG. 2 is a block diagram of an example system 200 for predicting whether a subject will respond to an ICI therapy, according to some embodiments of the technology described herein.

FIG. 3A is a flowchart of an illustrative process 300 for predicting, using cytometry data, whether a subject will respond to an ICI therapy, according to some embodiments of the technology described herein.

FIG. 3B is a flowchart of an illustrative process 350 for predicting, using cell population data, whether a subject will respond to an ICI therapy, according to some embodiments of the technology described herein.

FIG. 4A is an illustrative example of selecting a molecular functional (MF) profile type for a subject, according to some embodiments of the technology described herein.

FIG. 4B is an illustrative example of determining a G2 score for a blood sample using cell population data, according to some embodiments of the technology described herein.

FIG. 4C is an illustrative example of determining a G2 score for a blood sample using RNA expression data, according to some embodiments of the technology described herein.

FIG. 5A is a flowchart of an illustrative process for determining a G2 score for a blood sample, according to some embodiments of the technology described herein.

FIG. 5B and FIG. 5C are example plots showing the relationship between immunoprofile types and G2 score, according to some embodiments of the technology described herein.

FIG. 6A is a flowchart of an illustrative process 600 for determining an immunoprofile type for a subject using cytometry data, according to some embodiments of the technology described herein.

FIG. 6B is a flowchart of an illustrative process 620 for determining an immunoprofile type for a subject using RNA expression data, according to some embodiments of the technology described herein.

FIG. 6C is a flowchart of an illustrative process 640 for determining an immunoprofile type for a subject using cell population data, according to some embodiments of the technology described herein.

FIG. 7 is a flowchart of an illustrative process for determining cell composition percentages based on cell counts determined for a plurality of cells of a biological sample, according to some embodiments of the technology described herein.

FIG. 8A is a flowchart of an illustrative process 800 for identifying an MF profile type with which to associate an MF profile for a subject, in accordance with some embodiments of the technology described herein.

FIG. 8B is a flowchart of an illustrative process 820 for generating MF profile clusters using RNA expression data obtained from subjects having a particular type of cancer, in accordance with some embodiments of the technology described herein.

FIG. 9A is an example showing the segregation of blood samples into different immunoprofile types, according to some embodiments of the technology described herein.

FIG. 9B and FIG. 9C, are example bar plots showing that more subjects who were responsive to an ICI therapy had a Primed (G2) immunoprofile type rather than a non-G2 immunoprofile type, according to some embodiments of the technology described herein.

FIG. 10 show example correlations between response to an ICI therapy and values of tumor expression biomarkers, according to some embodiments of the technology described herein.

FIG. 11A is an example heatmap showing the segregation of tumor samples into different MF profile types, according to some embodiments of the technology described herein.

FIG. 11B and FIG. 11C are example bar plots showing that more subjects who were responsive to an ICI therapy had an immune-enriched molecular profile type rather than a non-immune-enriched profile type, according to some embodiments of the technology described herein.

FIG. 12A and FIG. 12B are example bar plots showing that more subjects who were responsive to an ICI therapy had an immune-enriched molecular profile type rather than a non-immune-enriched profile type, according to some embodiments of the technology described herein.

FIG. 12C and FIG. 12D are example bar plots showing that more subjects who were responsive to an ICI therapy had a Primed (G2) immunoprofile type rather than a non-G2 immunoprofile type, according to some embodiments of the technology described herein.

FIG. 12E and FIG. 12F are example bar plots showing that more subjects who were responsive to ICI therapy had an immune enriched molecular profile type and/or a G2 immunoprofile type, according to some embodiments of the technology described herein.

FIG. 13A shows an example correlation between response to an ICI therapy and predicted probability of therapeutic response for subjects in a validation cohort, according to some embodiments of the technology described herein.

FIG. 13B shows an example receiver operating characteristic (ROC) curve showing the performance of a statistical model used to predict therapeutic response of subjects in a validation cohort, according to some embodiments of the technology described herein.

FIG. 13C shows an example correlation between response to an ICI therapy and predicted probability of therapeutic response for subjects in a human papillomavirus negative head and neck squamous cell carcinomas (HPV-HNSCC) validation cohort, according to some embodiments of the technology described herein.

FIG. 13D shows an example ROC curve showing the performance of a statistical model used to predict therapeutic response of subjects in an HPV-HNSCC validation cohort, according to some embodiments of the technology described herein.

FIG. 14 is a schematic diagram of an illustrative computing device with which aspects described herein may be implemented.

FIG. 15A is an example showing the segregation of blood samples into different immunoprofile types, according to some embodiments of the technology described herein.

FIG. 15B shows an example Sankey plot showing the distribution of five immunotypes among responders and non-responders to nivolumab, according to some embodiments of the technology described herein.

FIG. 15C shows example box plots representing comparison of pre-treatment samples of responders (R) and non-responders (NR) to nivolumab, according to some embodiments of the technology described herein.

FIG. 15D is an example heatmap showing the segregation of tumor samples into different MF profile types, according to some embodiments of the technology described herein.

FIG. 15E and FIG. 15F are example bar plots showing that more subjects who were responsive to an ICI therapy had an immune-enriched molecular profile type rather than a non-immune-enriched profile type, according to some embodiments of the technology described herein.

### DETAILED DESCRIPTION

The inventors have developed techniques for predicting whether a subject will respond to an immune checkpoint inhibitor (ICI) therapy. In some embodiments, the techniques include: (a) obtaining RNA expression data for a tumor sample from the subject, (b) selecting, from among multiple molecular-functional (MF) profile types and using the RNA expression data, an MF profile type for the tumor sample, (c) obtaining cell population data (e.g., cytometry data) for a blood sample from the subject, (d) determining, using the cell population data, a G2 score for the blood sample, and (e) predicting, using a statistical model and based on the selected MF profile type and the G2 score, whether the subject will respond to the ICI therapy. The G2 score may be indicative of a likelihood that the blood sample is of a Primed (G2) immunoprofile type. In some embodiments, the ICI therapy is administered to the subject (e.g., if the subject is predicted to respond to it).

An “MF profile type” may refer to a tumor microenvironment (TME) having certain features including certain gene expression levels, gene group expression levels, molecular and cellular compositions, and/or biological processes. In some embodiments, a TME may be characterized or classified as one of four molecular functional (MF) profile types, herein identified as the first MF profile type, second MF profile type, third MF profile type, and fourth MF profile type. TMEs of the first MF profile type may also be described as “immune-enriched/fibrotic”; TMEs of the second MF profile type may also be described as “immune-enriched/non-fibrotic”; TMEs of the third MF profile type may also be described as “fibrotic”; TMEs of the fourth MF profile type may be described as “immune desert.” Aspects of MF profile types are described herein including at least in the section “MF profile types.”

An “immunoprofile type” of a blood sample may refer to one of a plurality of immunoprofile types that can be associated with the blood sample, the plurality of immunoprofile types differing by their cell composition percentages for one or more types of immune cells (e.g., one or more types of peripheral blood mononuclear cells (PBMCs)). In some embodiments, a blood sample may be characterized or classified as one of five immunoprofile types. The five immunoprofile types may be described as a Naive type (G1), a Primed type (G2), a Progressive type (G3), a Chronic type (G4), and a Suppressive type (G5). Aspects of immunoprofile types are described herein including at least in the section “Immunoprofile Types.”

The highly heterogenous nature of cancer and the complexity of the immune system present significant therapeutic challenges. For example, different patients diagnosed with the same cancer diagnosis may have different response to the same treatments such as, for example, an immunotherapy. This makes it challenging to predict whether a particular therapy will be effective for a subject (e.g., whether the subject will respond to the therapy).

In evaluating whether a subject will respond to an immunotherapy, conventional techniques have focused on limited aspects of the overall system that contributes to immunotherapy response. For example, some conventional techniques focus on characteristics of the local tumor microenvironment (TME) to determine whether a subject will respond to an immunotherapy. The TME is complex and includes many components that may affect how a subject will respond to an immunotherapy. Therefore, understanding the composition of the TME may be important for predicting how a subject will respond to an immunotherapy. However, there are many other components that interact with the TME that may affect how a subject will respond to an immunotherapy. Beyond the TME, the body's immune system includes a complex network of biological processes that may interact with the tumor and TME and affect how a subject will respond to an immunotherapy. While an evaluation of characteristics of the TME may indicate that the subject is likely to respond to an immunotherapy, characteristics of the immune system may hinder that response. Alternatively, while an evaluation of the TME may indicate that the subject is not likely to respond to an immunotherapy, characteristics of the immune system may promote a response. Therefore, by focusing on only limited aspects of the overall system (e.g., the TME), the conventional techniques fail to account for other factors that may contribute to immunotherapy response, resulting in weak or inaccurate predictions.

Accordingly, the inventors have developed techniques that address the above-described challenges associated with the conventional techniques for predicting whether a subject will respond to an ICI therapy. In some embodiments, the techniques include: (a) obtaining RNA expression data previously obtained from a tumor sample from the subject, (b) selecting, from among multiple molecular-functional (MF) profile types and using the RNA expression data, an MF profile type for the tumor sample, (d) obtaining cell population data (e.g., cytometry data) previously obtained from a blood sample from the subject, (e) determining, using the cell population data, a G2 score for the blood sample, and (f) predicting, using a statistical model and based on the selected MF profile type and the G2 score, whether the subject will respond to the ICI therapy.

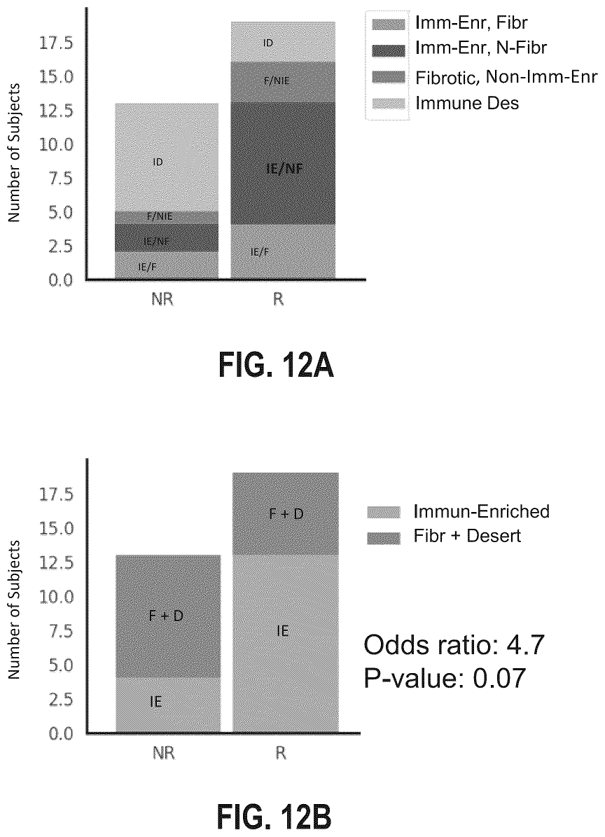

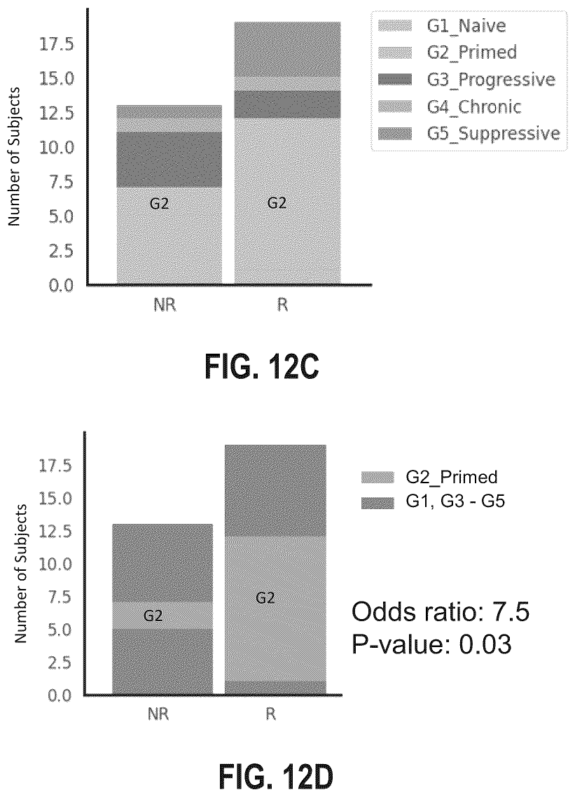

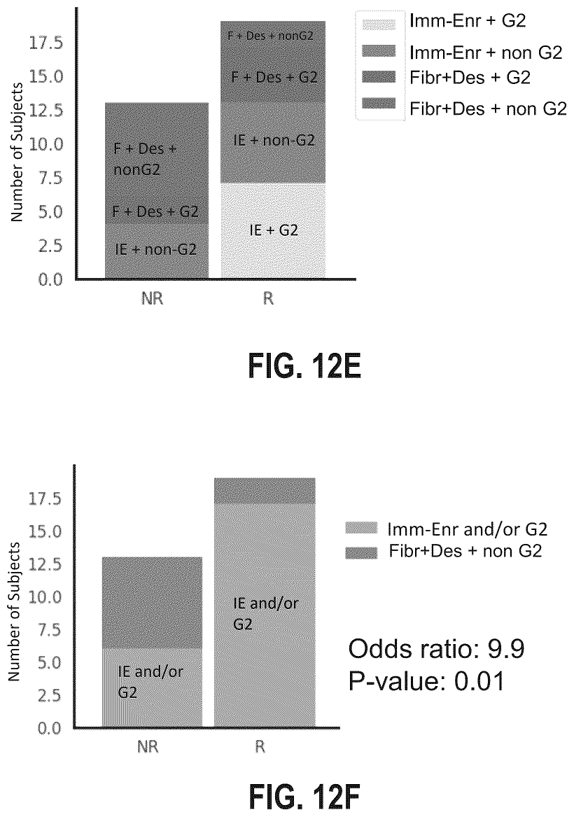

The techniques developed by the inventors are more comprehensive than conventional techniques because the prediction is based on characteristics of both molecular characteristics of a tumor sample (e.g., the MF profile type) and immune properties of a blood sample (e.g., the G2 score). Accordingly, the techniques account for characteristics of both the tumor microenvironment and the immune macroenvironment that may contribute to how a subject will respond to an ICI therapy. Because of this comprehensive approach, the techniques developed by the inventors can be used to obtain a more accurate and reliable prediction of whether the subject will respond to an ICI therapy. For example, FIGS. 12E-12F show that subjects having a certain combination of tumor and blood characteristics are more likely to be responsive to an ICI (e.g., nivolimumab) than subjects who do not have that combination of tumor and blood characteristics. When taken together, the combination of tumor microenvironment and immune macroenvironment characteristics increases prediction accuracy compared to when taken alone (FIGS. 12A-12D).

Furthermore, this is an improvement over previous work because previous work was focused on sub-classifying patients having the same cancer type, whereas this disclosure describes characteristics of different tumor microenvironments and immune properties that are common across samples from subjects having different cancer types; and therefore, may have pan-cancer utility in determining potentially effective therapeutics for a given patient.

Following below are descriptions of various concepts related to, and embodiments of, techniques for predicting whether a subject will respond to an ICI therapy. It should be appreciated that various aspects described herein may be implemented in any of numerous ways, as techniques are not limited to any particular manner of implementation. Examples of details of implementations are provided herein solely for illustrative purposes. Furthermore, the techniques disclosed herein may be used individually or in any suitable combination, as aspects of the technology described herein are not limited to the use of any particular technique or combination of techniques.

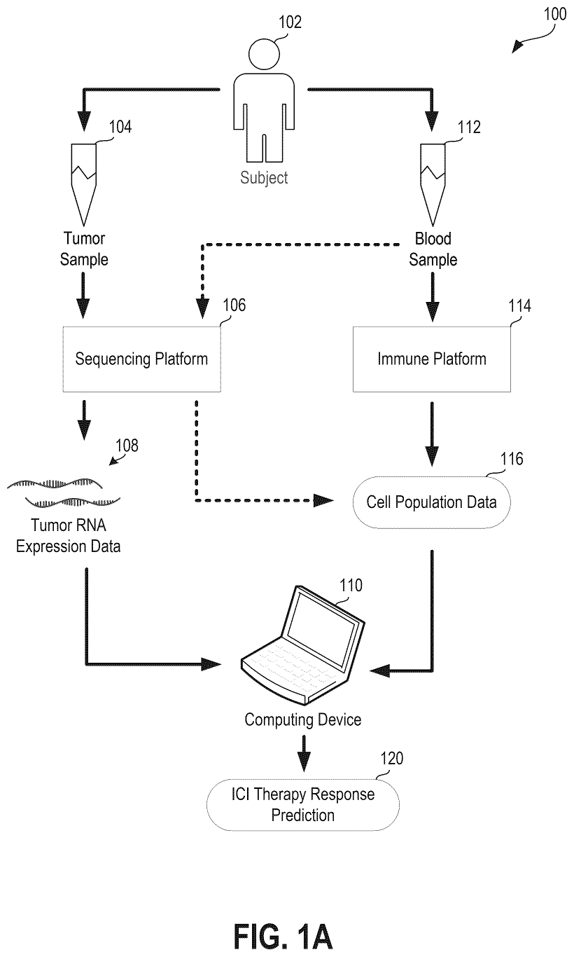

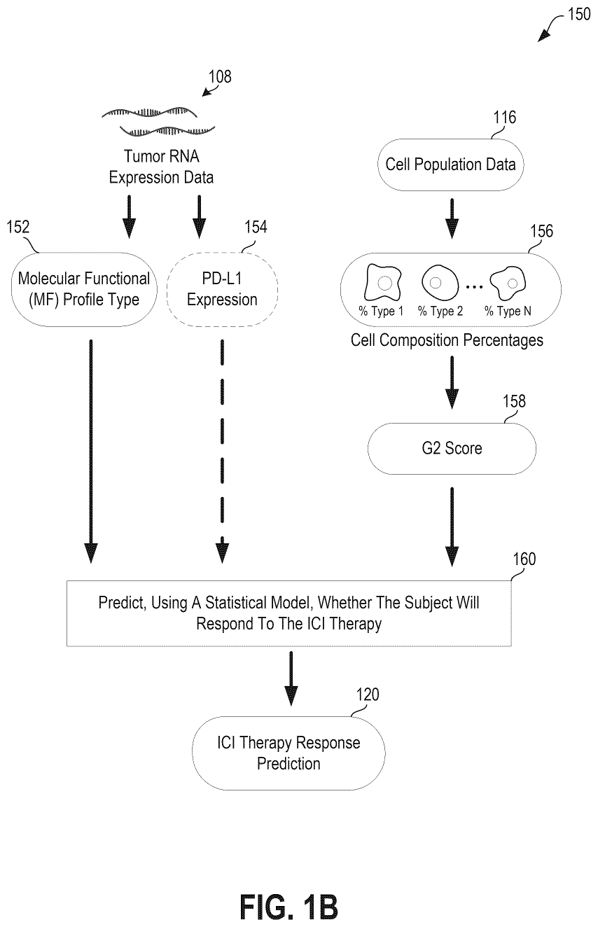

FIG. 1A is a diagram of an illustrative technique 100 for predicting whether a subject will respond to an immune checkpoint inhibitor therapy (ICI), according to some embodiments of the technology described herein. Technique 100 includes (a) obtaining RNA expression data 108 from a tumor sample 104 from the subject 102, (b) obtaining cell population data 116 from a blood sample 112 from the subject 102, and (c) processing the tumor RNA expression data 108 and the cell population data 116 using computing device 110 to obtain the ICI therapy response prediction 120. In some embodiments, technique 100 includes obtaining the tumor RNA expression data 108 by sequencing the tumor sample 104, respectively, using sequencing platform 106. In some embodiments, technique 100 includes obtaining the cell population data 116 by processing the blood sample 112 using the immune platform 114 and/or by sequencing the blood sample 112 using sequencing platform 106.

In some embodiments, aspects of the illustrative technique 100 may be implemented in a clinical or laboratory setting. For example, aspects of the technique 100 may be implemented on a computing device 110 that is located within the clinical or laboratory setting. In some embodiments, the computing device 110 may obtain tumor RNA expression data 108 and/or cell population data 116 from a sequencing platform 106 co-located with the computing device 110 within the clinical or laboratory setting. For example, the computing device 110 may be included in the sequencing platform 106. Additionally, or alternatively, the computing device 110 may obtain cell population data 116 from an immune platform 114 co-located with the computing device 110 within the clinical or laboratory setting. For example, the computing device 110 may be included in the immune platform 114. In some embodiments, the computing device 110 may indirectly obtain the RNA expression data and/or cell population data from a sequencing and/or immune platform located externally from or co-located with the computing device 110. For example, the computing device 110 may obtain RNA expression data and/or cell population data via at least one communication network, such as the Internet or any other suitable communication network(s), as aspects of the technology described herein are not limited in this respect.

In some embodiments, aspects of the illustrative techniques 100 may be implemented in a setting that is located externally from a clinical or laboratory setting. In this case, the computing device 110 may indirectly obtain RNA expression data and/or cell population data from a sequencing and/or immune platform located within or externally to a clinical or laboratory setting. For example, the RNA expression data and/or cell population data may be provided to the computing device 110 via at least one communication network, such as the Internet or any other suitable communication network(s), as aspects of the technology described herein are not limited in this respect.

In some embodiments, technique 100 includes obtaining a tumor sample 104 and a blood sample 112 from a subject. In some embodiments, the tumor sample 104 and/or blood sample 112 were previously-obtained from the subject 102. In some embodiments, the subject 102 has, is suspected of having, or is at risk of having cancer. In some embodiments, the cancer is a solid tumor. In some embodiments, the cancer is a non-hematological cancer. The cancer may be any suitable type of cancer, as aspects of the technology described herein are not limited in this respect. Nonlimiting examples of cancer types include melanoma, sarcomas, carcinomas, glioblastoma, gastric cancers, bladder cancers, follicular lymphoma or any other suitable types of cancer. For example, the subject 102 may have head and neck squamous cell carcinoma (HNSCC).

As shown in FIG. 1A, tumor RNA expression data 108 is obtained by processing a tumor sample 104 obtained for the subject 102. A tumor sample, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells. The origin, type, or preparation methods of the tumor sample 104 may include any of the embodiments relating to tumor samples described in the section “Biological Samples.”

Cell population data 116 is obtained by processing a blood sample 112 obtained for the subject 102. A blood sample, in some embodiments, refers to a sample comprising cells, e.g., cells from a blood sample. The blood sample can be any sample from which blood cell counts (e.g., immune cell counts, PBMC counts, etc.) can be obtained, including from whole cells or genetic material (e.g., RNA or DNA) derived therefrom. In some embodiments, the sample of blood comprises non-cancerous cells. In some embodiments, the sample of blood comprises precancerous cells. In some embodiments, the sample of blood comprises cancerous cells. In some embodiments, the sample of blood comprises blood cells. In some embodiments, the sample of blood comprises red blood cells. In some embodiments, the sample of blood comprises white blood cells. In some embodiments, the sample of blood comprises platelets. A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot. The origin, type, or preparation methods of the blood sample 112 may include any of the embodiments relating to blood samples described in the section “Biological Samples.”

In some embodiments, the tumor RNA expression data 108 and/or cell population data 116 is obtained using a sequencing platform 106 to obtain sequencing data. For example, the tumor RNA expression data 108 may be obtained by sequencing the tumor sample 104 using sequencing platform 106. Additionally or alternatively, the cell population data 116 may be obtained by sequencing the blood sample 112 using the sequencing platform 106. The sequencing platform 106 may include a next generation sequencing platform (e.g., Illumina®, Roche®, Ion Torrent®, etc.), any high-throughput or massively parallel sequencing platform, and/or a platform configured to perform sequencing techniques other than next generation sequencing (e.g., Sanger sequencing, microarrays, etc.). The sequencing data may comprise bulk RNA sequencing (RNA-seq) data, single cell RNA sequencing data (scRNA-seq), next generation sequencing (NGS) data, cellular indexing of transcriptomes and epitopes by sequencing (CITE-seq) data, DNA methylation data, and/or any sequencing data of any other suitable type, in any suitable format, and from any suitable source, as aspects of the technology described herein are not limited in this respect.

In some embodiments, the tumor RNA expression data 108 includes the sequencing data obtained from the sequencing platform 106 and/or data derived from the sequencing data obtained from sequencing platform 106. In some embodiments, the tumor RNA expression data 108 includes gene expression levels for one or more genes. In some embodiments, the tumor RNA expression data 108 is obtained by processing sequencing data obtained using the sequencing platform 106. This may be done in any suitable way and may involve expressing the bulk sequencing data in transcriptsper-million (TPM) units (or other units) and/or log transforming the RNA expression levels in TPM units. The origin, type, or preparation of the tumor RNA expression data 108 may include any of the embodiments described with respect to the section “Sequencing Data.”

The cell population data 116 may additionally or alternatively be obtained using an immune platform 114. For example, the cell population data 116 may be obtained by processing the blood sample 112 using the immune platform 114. An immune platform can be any assay and/or a system from which cell type counts can be obtained. For example, an immune platform can be any assay and/or system from which cell type counts can be obtained using cell type specific affinity reagents.

In some embodiments, the immune platform 114 includes a cytometry platform. For example, the cytometry platform may include any suitable flow cytometry platform. Flow cytometry may be performed using any suitable techniques such as, for example, the techniques described herein including in the section entitled “Flow Cytometry.” Additionally or alternatively, the cytometry platform may include any suitable mass cytometry platform. Mass cytometry may be performed using any suitable techniques such as, for example, the techniques described herein including in the section entitled “Mass Cytometry.” Additionally or alternatively, the cytometry platform may include any suitable spectral cytometry platform. Spectral cytometry may be performed using any suitable techniques such as, for example, the techniques described herein including in the section entitled “Spectral Cytometry.”

In some embodiments, the immune platform 114 includes a hematology analyzer. The hematology analyzer may be configured to count and differentiate between different types of cells in the blood sample 112. For example, the hematology analyzer may be configured to identify and count basophils, eosinophils, lymphocytes, monocytes, and/or neutrophils. The hematology analyzer may include a commercially available hematology analyzer, such as those available from Sysmex.

In some embodiments, the immune platform 114 includes a multiplexed immunofluorescence (MxIF) imaging platform. In some embodiments, the blood sample 112 is stained using one or more fluorescent markers, and the MxIF platform is configured to obtain immunofluorescence images of the blood sample 112. For example, the MxIF platform may include at least a microscope and a computing device configured to obtain the immunofluorescence images. MxIF imaging may be performed using any suitable techniques such as, for example, the techniques described herein including in the section entitled “MxIF Imaging.”

In some embodiments, the cell population data 116 includes information relating to a plurality of cells, for example, information relating to populations of immune cell types (e.g., PBMCs) of the subject. In some embodiments, the cell population data comprises information relating to the presence, absence, and/or relative amounts of at least some (or all) of the cells of the plurality of cells.

For example, the cell population data 116 may include sequencing data obtained from the sequencing platform 106 and/or data derived from the sequencing data obtained from sequencing platform 106. For example, the cell population data 116 may include bulk RNA-seq data, scRNA-seq, NGS data, CITE-seq data, and/or DNA methylation data. Additionally or alternatively, the cell population data 116 may include RNA expression data (“blood RNA expression data”). The RNA expression data may include gene expression levels for a plurality of genes. In some embodiments, the RNA expression data is obtained by processing sequencing data obtained using the sequencing platform 106. This may be done in any suitable way and may involve expressing bulk sequencing data in TPM units (or other units) and/or log transforming the RNA expression levels in TPM units. The origin, type, or preparation of the cell population data 116 may include any of the embodiments described with respect to the section “Sequencing Data.”

Additionally or alternatively, the cell population data 116 may include cytometry data generated by a cytometry protocol, and/or information that can be inferred or determined from the cytometry data. For example, the cytometry data may include flow cytometry data, cytometry by time-of-flight data (CyTOF), and/or spectral cytometry data.

Additionally or alternatively, the cell population data 116 may include one or more MxIF images and/or data derived therefrom. For example, information derived from MxIF images may include information that identifies the location of cells in the image(s) and/or the different types of cells in the blood sample 112.

In some embodiments, the computing device 110 is used to process the tumor RNA expression data 108 and/or cell population data 116, and/or blood RNA expression data 118 to determine the ICI response prediction 120 for the subject 102. The computing device 110 may be operated by a user such as a doctor, clinician, researcher, the subject 102, and/or any other suitable entity. For example, the user may provide the tumor RNA expression data 108 and/or cell population data 116 as input to the computing device 110 (e.g., by uploading a file), provide user input specifying processing or other methods to be performed using the RNA expression data and/or cell population data, and/or provide input specifying one or more clinical features associated the subject 102, the tumor sample 104, and/or the blood sample 112.

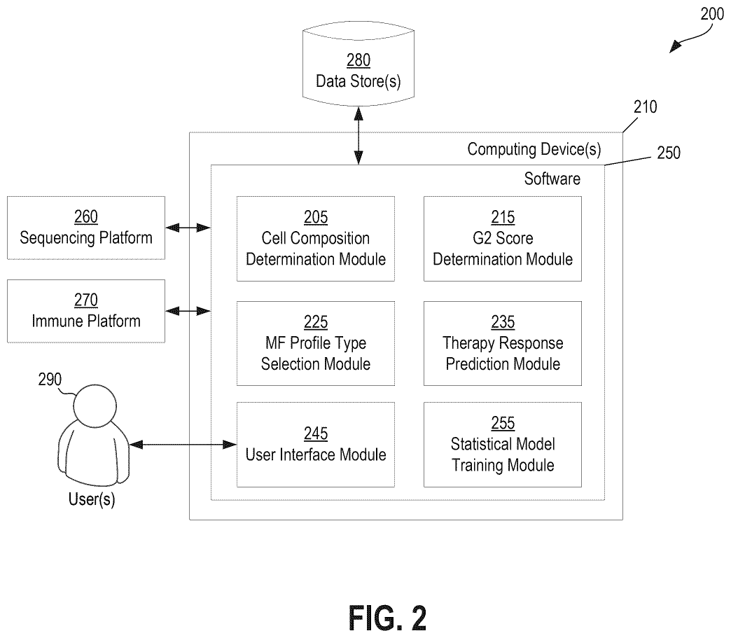

In some embodiments, software on the computing device 110 may be used to determine the ICI response prediction 120. An example of computing device 110 and such software is described herein including at least with respect to FIG. 2 (e.g., computing device(s) 210 and software 250).

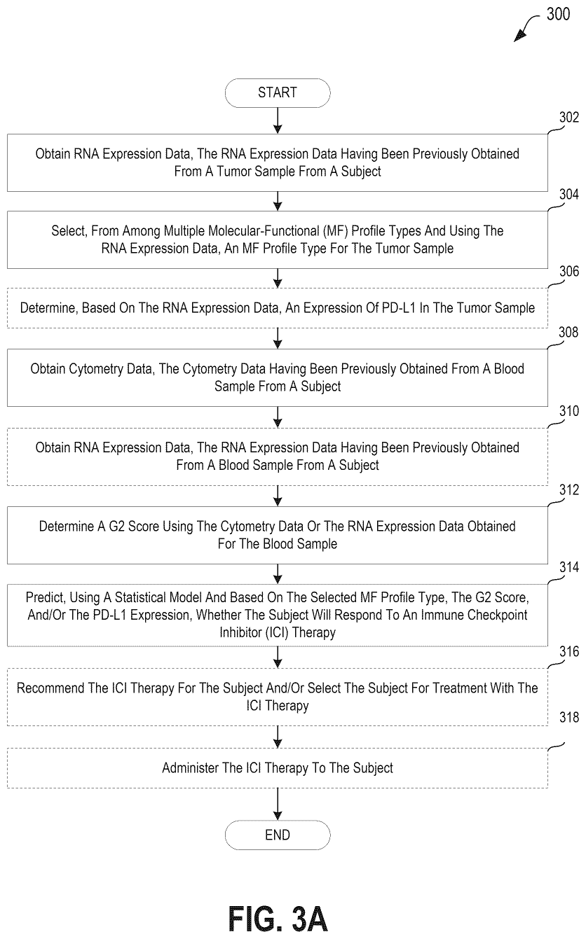

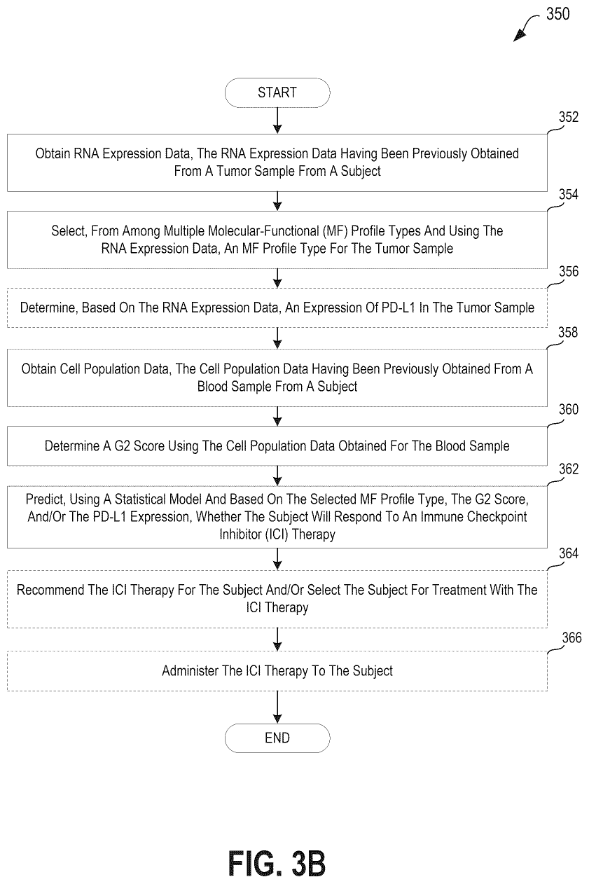

In some embodiments, software on the computing device 110 may be configured to process the tumor RNA expression data 108 and/or cell population data 116 to determine the ICI therapy response prediction 120. In some embodiments, this may include: (a) selecting, from among multiple molecular-functional (MF) profile types and using the tumor RNA expression data 108, an MF profile type for the tumor sample, (b) determining, using the cell population data 116, a G2 score for the blood sample, and (c) predicting, using a statistical model and based on the selected MF profile type and the G2 score, whether the subject will respond to the ICI therapy. Example techniques for predicting whether a subject will respond to an ICI therapy are described herein including at least with respect to FIG. 1B, FIG. 3A, and FIG. 3B.

In some embodiments, the ICI therapy response prediction 120 is indicative of whether or not the subject will respond to an ICI therapy. For example, in some embodiments, the ICI therapy response prediction 120 indicates a likelihood that the subject will respond to the ICI therapy. Additionally, or alternatively, the ICI response prediction 120 includes a binary output indicating whether or not the subject will respond to the ICI therapy. It should be appreciated, however, that the ICI therapy response prediction 120 may convey the prediction in any other suitable manner, as aspects of the technology described herein are not limited in this respect.

In some embodiments, the ICI therapy response prediction 120 may be used to determine whether to administer the ICI therapy to the subject. Techniques for administering a therapy to a subject are described herein including at least in the section “Therapies.”

The ICI therapy may include any therapy that inhibits one or more immune checkpoint mechanisms. Nonlimiting examples of immune checkpoint inhibitors include pembrolizumab, ipilimumab, nivolumab, cemiplimab, dostarlimab, atezolizumab, durvalumab, and avelumab. In some embodiments, the ICI therapy includes anti-PD-1 antibodies, anti-CTLA4 antibodies, and/or anti-PD-L1 antibodies. Examples of ICI therapies and techniques for administering ICI therapies are described herein including at least in the section “Therapies.”

In some embodiments, the computing device 110 is configured to generate an output indicating the ICI therapy response prediction 120. In some embodiments, the output of the computing device 110 is stored (e.g., in memory), displayed via a user interface, transmitted to one or more other devices, used to generate a report, or otherwise processed using any other suitable techniques, as aspects of the technology described herein are not limited in this respect. For example, the computing device 110 may be displayed via a graphical user interface (GUI) of a computing device (e.g., computing device 110).

In some embodiments, the output of the computing device 110 may be in the form of a report, such as a report including an indication of the ICI therapy response prediction 120. The generated report can provide a summary of information, so that a clinician can determine whether to administer a therapy to the subject. The report as described herein may be a paper report, an electronic record, or a report in any format that is deemed suitable in the art. The report may be shown and/or stored on a computing device known in the art (e.g., a handheld device, desktop computer, smart device, website, etc.). The report may be shown and/or stored on any device that is suitable as understood by a skilled person in the art.

In some embodiments, the methods and reports disclosed herein may include database management for the keeping of generated reports. For instance, the methods as disclosed herein can create a record in a database for the subject 102 and populate the specific record with data for the subject 102. In some embodiments, the generated report can be provided to the subject 102, clinicians, doctors, researchers, or any other suitable entity. In some embodiments, a network connection can be established to a server computer that includes the data and report for receiving or outputting. In some embodiments, the receiving and outputting of the data or report can be requested from the server computer.

In some embodiments, the computing device 110 includes one or multiple computing devices. In some embodiments, when the computing device 110 includes multiple computing devices, each of the computing devices may be used to perform the same process or processes. For example, each of the multiple computing devices may include software used to implement process 300 shown in FIG. 3A and/or process 350 shown in FIG. 3B. In some embodiments, when the computing device 110 includes multiple computing devices, the computing devices may be used to perform different processes or different aspects of a process. For example, one computing device may include software used to select an MF profile type for the tumor sample, while a different computing device may include software used to determine a G2 score for the blood sample.

In some embodiments, when the computing device 110 includes multiple computing devices, the multiple computing devices may be configured to communicate via at least one communication network such as the Internet or any other suitable communication network(s), as aspects of the technology described herein are not limited in this respect. For example, one computing device may be configured to determine a G2 score for the blood sample, and then provide the G2 score to one or more other computing devices via the communication network.

FIG. 1B is a diagram of an illustrative technique 150 for predicting whether a subject (e.g., subject 102 in FIG. 1A) will respond to an ICI therapy, according to some embodiments of the technology described herein. Technique 150 includes, at act 160, predicting, using a statical model and based on a molecular functional (MF) profile type 152, a G2 score 158, and/or an expression of PD-L1 154, whether the subject will respond to an ICI therapy to obtain the ICI therapy response prediction 120. In some embodiments, the MF profile type 152 and the PD-L1 expression 154 are determined using the tumor RNA expression data 108. In some embodiments, the G2 score 158 is determined by (a) determining cell composition percentages 156 using the cell population data 116, and (b) using the cell composition percentages 156 to determine the G2 score 158. As described herein, including at least with respect to FIG. 1A, illustrative techniques 150 may be implemented using a computing device such as computing device 110 shown in FIG. 1A.

As shown in FIG. 1B, technique 150 includes selecting an MF profile type 152 for a tumor sample (e.g., tumor sample 104 shown in FIG. 1A) using the tumor RNA expression data 108 obtained for the tumor sample. In some embodiments, the MF profile type 152 is selected from among multiple MF profile types such as, for example, an immune-enriched/fibrotic type, an immune-enriched/non-fibrotic type, a fibrotic type, or an immune desert type. Aspects of MF profile types are described in the section “MF Profile Types.”

In some embodiments, selecting an MF profile type 152 for the tumor sample includes determining an MF profile for the tumor sample and selecting the MF profile type based on the MF profile determined for the subject. An “MF profile” as described herein, refers to biological processes that are present within and/or surrounding the tumor. Related compositions and processes present within and/or surrounding a tumor are presented in gene groups of an MF profile. A “gene group,” as described herein, refers to a set of genes that is associated with related compositions and processes present within and/or surrounding a tumor.

In some embodiments, determining the MF profile for the tumor sample includes determining a set of expression levels for a respective set of gene groups that includes one or more gene groups. The MF profile may be determined for a subject having any type of cancer. The MF profile may be determined using any number of gene groups that relate to compositions and processes present within and/or surrounding the subject's tumor. Gene group expression levels may be calculated for the gene groups. A gene group expression level, may refer to a score that quantifies whether the genes in a gene group are over-represented or over-expressed in a sample. For example, a gene group expression level may be calculated as a gene set enrichment (GSEA) score for the gene group. Further aspects relating to determining MF profiles are described herein including at least in the section titled “MF Profiles”.

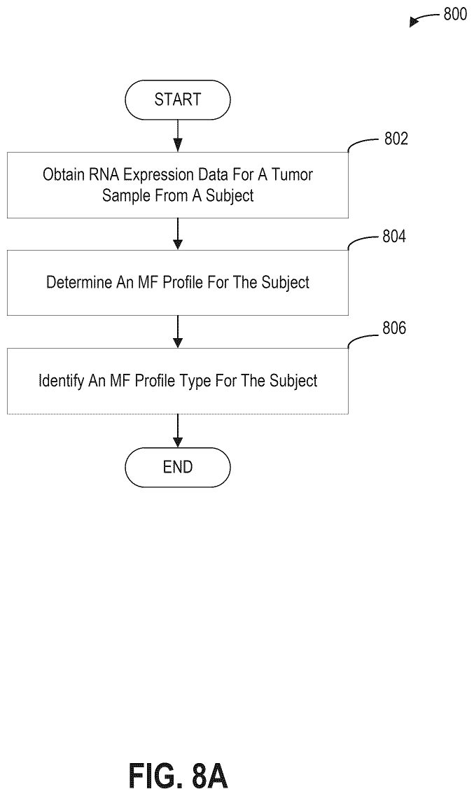

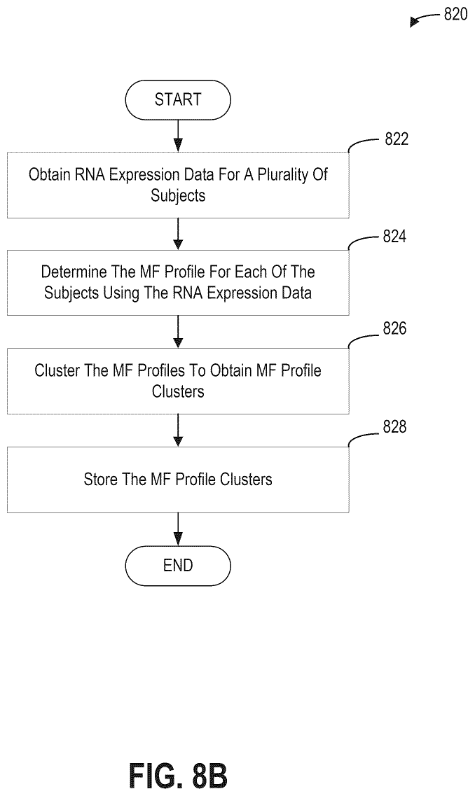

In some embodiments, selecting an MF profile type based on an MF profile determined for the tumor sample includes identifying a cluster with which the MF profile is associated. For example, different MF profile clusters may correspond to the different MF profile types. Therefore, the terms “MF profile clusters” and “MF profile types” are used herein interchangeably unless context indicates otherwise. In some embodiments, an MF profile may be associated with one of the MF profile clusters using a similarity metric (e.g., by associating the MF profile with the MF profile cluster whose centroid is closest to the MF profile according to the similarity metric). In some embodiments, a statistical classifier (e.g., k-means classifier or any other suitable type of statistical classifier) may be trained to classify the MF profile as belonging to one or multiple of the MF profile clusters. For example, the statistical classifier may be trained by clustering MF profiles from a plurality of training samples from a plurality of subjects to obtain the MF profile clusters. Further aspects relating to generating MF profile clusters and selecting MF profile types are described herein including at least in the section “Selecting MF Profile Types” and with respect to FIG. 8A and FIG. 8B.

In some embodiments, the MF profile type 152 is encoded. The encoding may be binary or multilevel (e.g., a different encoding may be generated for respective groups of MF profile types or for each MF profile type). The MF profile type may be encoded using any suitable encoding techniques, as aspects of the technology described herein are not limited in this respect. For example, encoding the MF profile type 152 may include assigning a value to the MF profile type based on whether it is of the immune-enriched/fibrotic MF profile type, the immune-enriched/non-fibrotic MF profile type, the fibrotic MF profile type (e.g., fibrotic/non-immune-enriched), and/or the immune desert type (e.g., non-fibrotic/non-immune enriched). For example, a first value (e.g., 1) may be assigned to the MF profile type 152 when it is the immune-enriched/fibrotic MF profile type or the immune-enriched/non-fibrotic MF profile type, and a second value (e.g., 0) may be assigned to the MF profile type 152 when it is the fibrotic type or immune desert type.

Technique 150 includes determining cell composition percentages 156 for a blood sample (e.g., blood sample 112 shown in FIG. 1A) using the cell population data 116 obtained for the blood sample.

In some embodiments, the cell population data is processed to obtain cell composition percentages for at least some cell types of a plurality of cell types in the blood sample. For example, the cell population data may be processed to obtain cell composition percentages for at least some (e.g., all) of the cell types listed in Table 2, Table 3, and/or Table 4. Additionally, or alternatively, the cell population data may be processed to obtain a cell composition percentage of peripheral mononuclear cells (PBMCs) in the blood sample (e.g., the total percentage of PBMCs of all types, or a sum of percentages of PBMCs of a plurality of types). Example techniques for determining cell composition percentages for cell types in a blood sample are described herein including at least in the section entitled “Cell Composition Percentages.”

In some embodiments, the cell composition percentages 156 are used to determine a G2 score for the blood sample. For example, the cell composition percentages determined by processing the cell population data 116 may be used to determine the G2 score. In some embodiments, the G2 score is a numerical value that separates samples of the G2 immunoprofile type from samples of non-G2 immunoprofile types (e.g., G1, G3, G4, and G5). For example, the G2 score may be a probability that the blood sample is of a G2 immunoprofile type. In some embodiments, the G2 score is a value between 0 and 1.

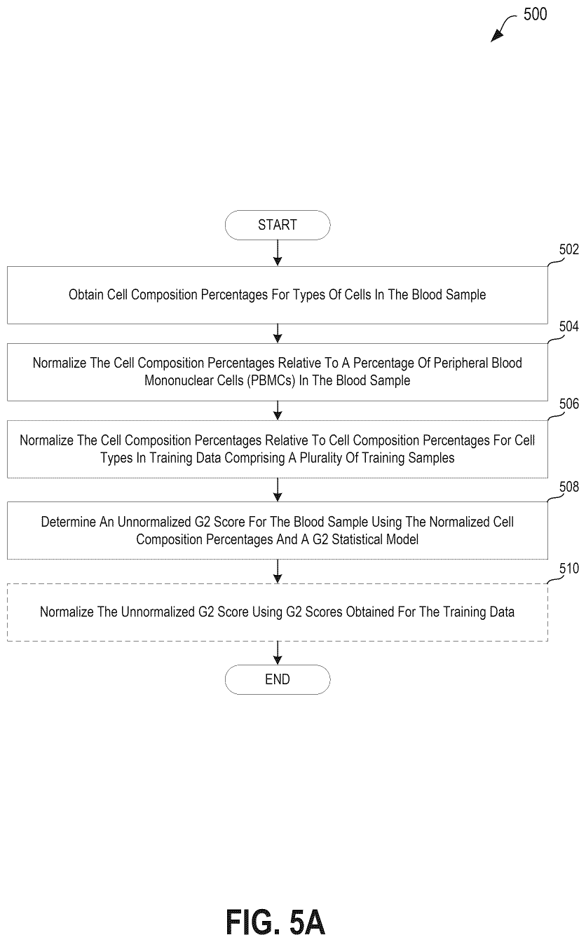

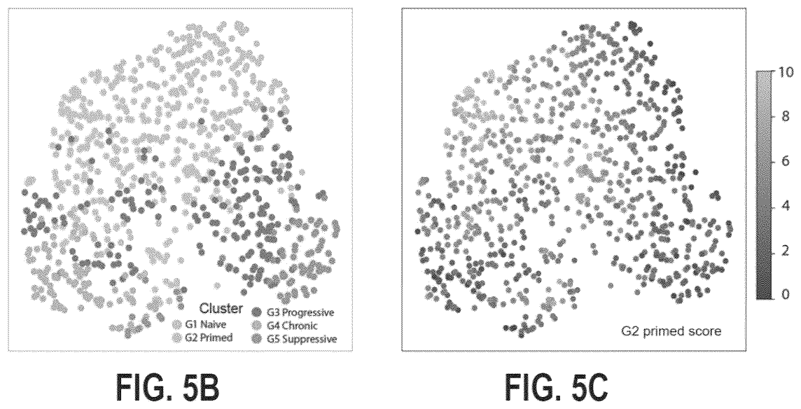

In some embodiments, determining a G2 score includes (a) normalizing the cell composition percentages relative to a percentage of PBMCs in the blood sample (e.g., the total percentage of PBMCs of all types, or a sum of percentages of PBMCs of a plurality of types), (b) normalizing the cell composition percentages with respect to corresponding cell composition percentages in training data obtained comprising a plurality of training samples, (c) determining an (unnormalized) G2 score for the blood sample using the normalized cell composition percentages and a G2 statistical model, and (c) (optionally) normalizing the (unnormalized) G2 score using G2 scores obtained for the training samples. Aspects of determining a G2 score for a subject using cell composition percentages are described herein including at least in the section “Immunoprofile Type Scores” and with respect to FIG. 5A.

In some embodiments, technique 150 includes determining an expression of PD-L1 154 for the tumor sample (e.g., tumor sample 104 shown in FIG. 1A) using the tumor RNA expression data 108. For example, this may include determining an expression level of CD274. In some embodiments, the expression level is expressed in TPM units. In some embodiments, the expression level is normalized. For example, the expression level may be normalized relative to a value such as, for example, a value associated with a cohort. For example, the expression level may be normalized relative to an expression level corresponding to a predetermined percentile of a distribution of PD-L1 expression levels measured for subjects in a cohort (e.g., a cohort of tumor samples). Additionally, or alternatively, the expression level may be normalized relative to a maximum value of a distribution of PD-L1 expression levels measured for a cohort. The normalization may be performed in any suitable manner as aspects of the technology described herein are not limited in this respect.

As shown in FIG. 1B, technique 150 includes predicting, based on the MF profile type 152, G2 score 158, and (optionally) the PD-L1 expression 154, whether the subject will respond to an ICI therapy. In some embodiments, predicting the therapeutic response 120 includes determining a score. The score may be expressed as a function of the MF profile type 152 (e.g., the encoded MF profile type), the G2 score 158, and/or the PD-L1 expression 154. The score may be calculated using a weighted sum of a plurality of predictors comprising the MF profile type 152, G2 score 158, and optionally PD-L1 expression 154. The predictors in the weighted sum may be weighted by predetermined coefficients. The predictors may be weighted by coefficients that have been previously determined using training data comprising values of the predictors and known response to ICI for a plurality of subjects. For example, coefficients may be or may have been previously estimated by based on training data (e.g., by performing a regression analysis on the training data). For example, the training data may include, for each of a plurality of training subjects, values for each of the predictors and a known therapeutic response (e.g., whether the subject is considered to have responded to ICI or not) for each of the training subjects. In some embodiments, the score is compared to a threshold to determine whether or not the subject will respond to the ICI therapy. For example, if the score is greater than or equal to the threshold, then the subject is predicted to be responsive to the ICI therapy. The threshold may be determined based on results of performing the regression analysis used to estimate coefficients (e.g., for the MF profile type 152, G2 score 158, and/or PD-L1 expression 154). For example, performance metrics (e.g., F1 score, positive predictive value, negative predictive value, etc.) used for evaluating the performance of the regression analysis in distinguishing between responsive and non-responsive subjects may be used to determine the threshold.

In some embodiments, a statistical model is used to predict whether the subject will respond to the ICI therapy based on the MF profile type 152, G2 score 158, and/or PD-L1 expression 154. The statistical model may include any suitable statistical model. A suitable statistical model may be any multivariate model that can be used to classify an observation comprising values for a plurality of predictive variables (e.g., MF profile type, G2 score, PD-L1 expression level, etc.) between two or more classes (e.g., classify a sample as responsive/non-responsive). For example, the statistical model may be a generalized linear model (e.g., a linear regression model, a logistic regression model, a probit regression model, etc.). It should be appreciated that, in some embodiments, the statistical model may not be a generalized linear model and may be a different type of statistical model such as, for example, a random forest regression model, a neural network, a support vector machine, a Gaussian mixture model, a hierarchical Bayesian model, and/or any other suitable statistical model, as aspects of the technology described herein are not limited to using generalized linear models for the predicting whether a subject with respond to an ICI therapy. In some embodiments, the statistical model is a classifier trained to classify subjects between a responsive and a non-responsive class. Techniques for processing one or more predictors using a statistical model are described herein including at least with respect to act 314 of process 300 shown in FIG. 3A and act 362 of process 350 shown in FIG. 3B.

FIG. 2 is a block diagram of an example system 200 for predicting whether a subject will respond to an ICI therapy, according to some embodiments of the technology described herein. System 200 includes computing device(s) 210 configured to have software 250 execute thereon to perform various functions in connection with predicting whether a subject will respond to an ICI therapy. In some embodiments, software 250 includes a plurality of modules. A module may include processor-executable instructions that, when executed by at least one computer hardware processor, cause the at least one computer hardware processor to perform the function(s) of the module. Such modules are sometimes referred to herein as “software modules,” each of which includes processor executable instructions configured to perform one or more processes, such as process 300 described herein including at least with respect to FIG. 3A and/or process 350 shown in FIG. 3B.

The computing device(s) 210 may be operated by one or more user(s) 290. For example, the user(s) 290 may include one or more individuals who are treating and/or studying (e.g., doctors, clinicians, researchers, etc.) the subject. Additionally, or alternatively, user(s) 290 may include the subject. In some embodiments, the user(s) 290 may provide, as input to the computing device(s) 210 (e.g., by uploading one or more filed, by interacting with a user interface of the computing device(s) 210, etc.) RNA expression data obtained for a tumor sample (e.g., previously obtained for a tumor sample), RNA expression data obtained for a blood sample (e.g., previously obtained for a blood sample), and/or cell population data obtained for a blood sample (e.g., previously obtained for a blood sample). Additionally, or alternatively, the user(s) 290 may provide input specifying processing or other methods to be performed on the RNA expression data and/or cell population data. Additionally, or alternatively, the user(s) 290 may access results of processing the RNA expression data and/or cell population data. For example, the user(s) 290 may access results of predicting whether the subject will respond to an ICI therapy.

As shown in FIG. 2, software 250 includes multiple software modules for predicting whether a subject will respond to an ICI therapy. Some software modules include a cell composition determination module 205, a G2 score determination module 215, an MF profile type selection module 225, and a therapy response prediction module 235.

In some embodiments, the cell composition determination module 205 obtains cell population data (e.g., cell population data 116 shown in FIG. 1A and FIG. 1B) from sequencing platform 260, immune platform 270, the user(s) 290 (e.g., the user(s) uploading the cell population data), and/or data store(s) 280.

In some embodiments, the cell composition determination module 205 is configured to determine cell composition percentages for cell types in the blood sample by processing cell population data obtained for the blood sample. In some embodiments, the cell composition determination module 205 is configured to apply one or more of the example techniques described herein for determining cell composition percentages, such as any of those described herein including at least in the section entitled “Cell Composition Percentages.” For example, the cell composition determination module 205 may be configured to apply one or more machine learning models to the cell population data to obtain the cell composition percentages.

In some embodiments, the G2 score determination module 215 obtains cell composition percentages (e.g., cell composition percentages 156 in FIG. 1B) from cell composition determination module 205, data store(s) 280, and/or user(s) 290 (e.g., the user(s) uploading the cell composition percentages). In some embodiments, the G2 score determination module 215 obtains one or more G2 score statistical models from statistical model training module 255, data store(s) 280, and/or user(s) 290 (e.g., the user(s) uploading the statistical model(s)).

In some embodiments, the G2 score determination module 215 is configured to process cell composition percentages for cell types in the blood sample to determine a G2 score for the blood sample. In some embodiments, determining the G2 score includes (a) normalizing the cell composition percentages for the cell types relative to the percentage of PBMCs (e.g., the total percentage of PBMCs of all types, or a sum of percentages of PBMCs of a plurality of types) in the blood sample, (b) normalizing the cell composition percentages relative to corresponding cell composition percentages in training data comprising a plurality of training samples, (c) determining an unnormalized G2 score for the blood sample using the normalized cell composition percentages, and (d) normalizing the unnormalized G2 score using G2 scores obtained for the training data. Example techniques for determining a G2 score are described herein including at least with respect to FIG. 5 and in the section “Immunoprofile Type Scores.”

In some embodiments, the MF profile type selection module 225 obtains RNA expression data (e.g., tumor RNA expression data 108 in FIG. 1A and FIG. 1B) from sequencing platform 260, the user(s) 290 (e.g., the user(s) uploading the RNA expression data), and/or data store(s) 280.

In some embodiments, the MF profile type selection module 225 is configured to process RNA expression data obtained for a tumor sample from the subject to select an MF profile type for the tumor sample. This includes, in some embodiments, processing the RNA expression data to determine an MF profile for the tumor sample and selecting the MF profile type based on the determined MF profile. Examples of MF profile types are described in the section “MF Profile Types.” Examples for selecting an MF profile type are described herein including at least with respect to FIG. 8A, FIG. 8B, and in the sections “Selecting MF Profile Types” and “MF Profiles.”

In some embodiments, therapy response prediction module 235 obtains an MF profile type from the MF profile type selection module 225, data store(s) 280, and/or user(s) 290 (e.g., by the user(s) uploading the MF profile type). In some embodiments, therapy response prediction module 235 obtains a G2 score from the G2 score determination module 215, data store(s) 280, and/or user(s) 290 (e.g., the user(s) uploading the G2 score). In some embodiments, therapy response prediction module 235 obtains PD-L1 expression level(s) (e.g., PD-L1 expression 154 in FIG. 1B) from sequencing platform 260, data store(s) 280, and/or user(s) (e.g., the user(s) uploading the PD-L1 expression level(s)). In some embodiments, the therapy response prediction module 235 is configured to obtain one or more statistical models from statistical model training module 255, data store(s) 280, and/or user(s) 290 (e.g., the user(s) uploading the statistical model(s).

In some embodiments, the therapy response prediction module 235 is configured to predict whether or not a patient will respond to an ICI therapy. In some embodiments, to obtain the prediction, the therapy response prediction module 235 is configured to process an MF profile type selected for a tumor sample from the subject, a G2 score determined for a blood sample from the subject, and/or an expression of PD-L1 in the tumor sample from the subject. In some embodiments, the processing includes processing the MF profile type, the G2 score, and/or the PD-L1 expression using one or more statistical model(s) to obtain the prediction. Example techniques for predicting whether or not a subject will respond to an ICI therapy are described herein including at least with respect to act 314 of process 300 shown in FIG. 3A and act 362 of process 300 shown in FIG. 3B.

In some embodiments, software 250 further includes user interface module 245. User interface module 245 may be configured to generate a graphical user interface (GUI) through which the user may provide input and view information generated by software 250. For example, in some embodiments, the user interface module 245 may be a webpage or web application accessible through an Internet browser. In some embodiments, the user interface module 245 may generate a graphical user interface (GUI) of an app executing on the user's mobile device. In some embodiments, the user interface module 245 may generate a GUI on a sequencing platform, such as sequencing platform 260. In some embodiments, the user interface module 245 may generate a number of selectable elements through which a user may interact. For example, the user interface module 245 may generate dropdown lists, checkboxes, text fields, or any other suitable element.

In some embodiments, the user interface module 245 is configured to generate a GUI including one or more results of predicting whether a subject will respond to an ICI therapy. For example, the GUI may include an indication of the response prediction. Additionally, or alternatively, in some embodiments, the GUI may include an indication of the MF profile type selected for the subject, the G2 score determined for the subject, and/or the PD-L1 expression level determined for the subject. It should be appreciated that the GUI may include any other suitable information, displayed in any suitable manner, as aspects of the technology described herein are not limited in this respect.

As shown in FIG. 2, system 200 also includes sequencing platform 260. In some embodiments, sequencing data (e.g., RNA expression data, cell population data, etc.) is obtained from the sequencing platform 260. For example, the cell composition determination module 205, MF profile type selection module 225, and/or therapy response prediction module 235 may obtain (either pull or be provided) the sequencing data from the sequencing platform 260. The sequencing platform 260 may be one of any suitable type such as, for example, any of the sequencing platforms described herein including at least with respect to FIG. 1A and with respect to the section “Sequencing Data.”

System 200 further includes immune platform 270. In some embodiments, cell population data is obtained from the immune platform 270. For example, the cell composition determination module 105 may obtain (either pull or be provided) the cell population data from the immune platform 270. The immune platform 270 may be one of any suitable type such as, for example, any of the immune platforms described herein including at least with respect to FIG. 1A and with respect to the sections “Flow Cytometry” and “Mass Cytometry.” System 200 further includes data store(s) 280. In some embodiments, data store(s) 280 stores RNA expression data that was previously obtained for one or more subjects (e.g., using sequencing platform 260). Additionally, or alternatively, data store(s) 280 may store cell population data that was previously obtained for one or more subject(s) (e.g., using immune platform 270). Additionally, or alternatively, data store(s) 280 may store cell composition percentages (e.g., cell composition percentages determined using cell composition determination module 205). Additionally, or alternatively, data store(s) 280 may store MF profiles and/or MF profile types determined for one or more subject(s) (e.g., using MF profile type selection module 225). Additionally, or alternatively, data store(s) 280 may store G2 score(s) determined for one or more subject(s) (e.g., using G2 score determination module 215). Additionally, or alternatively, data store(s) 280 may store therapy response prediction(s) for one or more subject(s) (e.g., using the therapy response prediction module 235). Additionally, or alternatively, data store(s) 280 may store one or more trained statistical model(s) (e.g., trained using statistical model training module 255). It should be appreciated that the data store(s) 280 may store any other suitable type of information, as aspects of the technology described herein are not limited in this respect.

The data store(s) 280 may be of any suitable type (e.g., database system, multi-file, flat file, etc.) and may store data in any suitable way in any suitable format, as aspects of the technology described herein are not limited in this respect. The data store(s) 280 may be part of or external to the computing device(s) 210.

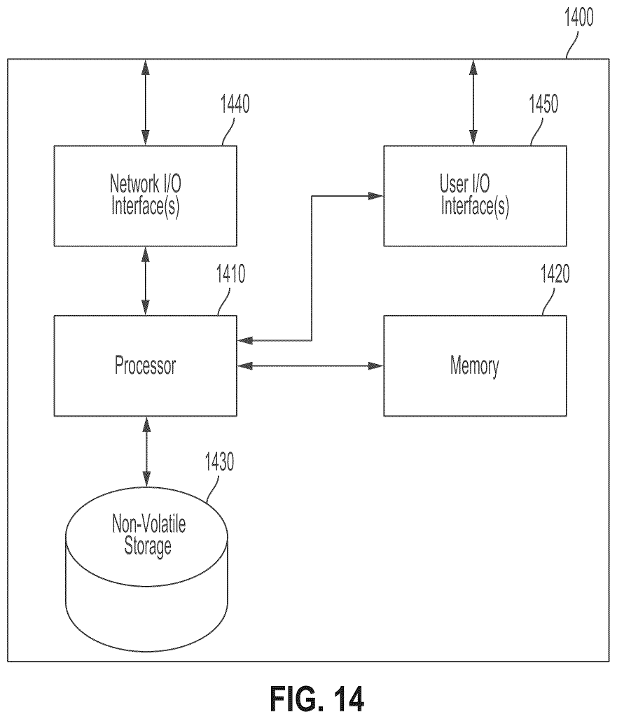

FIG. 3A is a flowchart of an illustrative process 300 for predicting whether a subject will respond to an ICI therapy, according to some embodiments of the technology described herein. One or more acts (e.g., all acts) of process 300 may be performed automatically by any suitable computing device(s). For example, the act(s) may be performed by a laptop computer, a desktop computer, one or more servers, in a cloud computing environment, computing device 1400 as described herein including with respect to FIG. 14, and/or in any other suitable way.

At act 302, RNA expression data is obtained for a tumor sample from a subject. In some embodiments, the RNA expression data was previously obtained for the tumor sample. Thus, in some embodiments, obtaining the RNA expression data may include accessing the data (e.g., from a memory, over a network, via a file being provided via an appropriate interface, etc.). For example, the RNA expression data may be obtained from a data store, such as data store(s) 280 shown in FIG. 2, and/or from user(s) (e.g., user(s) 290 shown in FIG. 2) providing a file including the segment data via an appropriate interface, such as user interface module 245 shown in FIG. 2.

In additional or alternative embodiments, obtaining the RNA expression data includes processing the tumor sample to obtain the RNA expression data. For example, the tumor sample may be processed using a sequencing platform (e.g., sequencing platform 106 in FIG. 1A, sequencing platform 260 in FIG. 2).

In some embodiments, the RNA expression data includes expression levels for one or more genes. For example, the RNA expression data may include expression levels for genes in one or more gene groups. Example gene groups are described herein including at least in the section “MF Profiles.” Additionally, or alternatively, the RNA expression data may include an expression level of PD-L1. The origin, type, or preparation of the RNA expression data may include any of the embodiments described herein including at least with respect to FIG. 1A and with respect to the section “Sequencing Data.”

At act 304, an MF profile type is selected for the tumor sample from among multiple MF profile types using the RNA expression data obtained at act 302. In some embodiment, the MF profile type is selected by determining an MF profile for the tumor sample using the RNA expression data obtained at act 302, and selecting the MF profile type based on the MF profile. In some embodiments, selecting an MF profile type based on an MF profile determined for the tumor sample includes identifying an MF profile cluster with which the MF profile is associated. For example, different MF profile clusters may correspond to the different MF profile types. In some embodiments, an MF profile may be associated with one of the MF profile clusters using a similarity metric (e.g., by associating the MF profile with the MF profile cluster whose centroid is closest to the MF profile according to the similarity metric). In some embodiments, a statistical classifier (e.g., k-means classifier or any other suitable type of statistical classifier) may be trained to classify the MF profile as belonging to one or multiple of the MF clusters. Further aspects relating to generating MF profile clusters and selecting MF profile types are described herein including at least in the section “Selecting MF Profile Types” and with respect to FIG. 8A and FIG. 8B.

In some embodiments, the MF profile type is encoded. For example, the MF profile type may be encoded using any suitable encoding techniques, as aspects of the technology described herein are not limited in this respect. For example, encoding the MF profile type may include assigning a value to the MF profile type based on whether it is of the immune-enriched/fibrotic MF profile type, the immune-enriched non-fibrotic MF profile type, the fibrotic MF profile type (e.g., fibrotic/non-immune-enriched), and/or the immune desert type (e.g., non-fibrotic/non-immune-enriched). For example, a first value (e.g., 1) may be assigned to the MF profile type 152 when it is the immune-enriched/fibrotic MF profile type or the immune-enriched non-fibrotic MF profile type, and a second value (e.g., 0) may be assigned to the MF profile type 152 when it is the fibrotic type or immune desert type.

At (optional) act 306, an expression of PD-L1 in the tumor sample is determined using the RNA expression data obtained at act 302. In some embodiments, the expression of PD-L1 is included in the RNA expression data obtained at act 302. In some embodiments, an unnormalized expression of PD-L1 is included in the RNA expression data and determining the expression of PD-L1 at act 306 includes determining a normalized expression of PD-L1. The normalizing may be performed using any suitable techniques, as aspects of the technology described herein are not limited to any particular normalization techniques. For example, the expression level may be expressed in TPM units. Additionally, or alternatively, the expression level may be normalized relative to a value such as, for example, a value associated with a cohort (e.g., a cohort of tumor samples). For example, the expression level may be normalized relative to an expression level corresponding to a predetermined percentile of a distribution of PD-L1 expression levels measured for subjects in a cohort (e.g., a cohort of tumor samples). Additionally, or alternatively, the expression level may be normalized relative to a maximum value of a distribution of PD-L1 expression levels measured for a cohort.

At act 308, cytometry data is obtained for a blood sample from the subject. In some embodiments, the cytometry data was previously obtained for the blood sample. Thus, in some embodiments, obtaining the cytometry data may include accessing the data (e.g., from a memory, over a network, via a file being provided via an appropriate interface, etc.). For example, the cytometry data may be obtained from a data store, such as data store(s) 280 shown in FIG. 2, and/or from user(s) (e.g., user(s) 290 shown in FIG. 2) providing a file including the segment data via an appropriate interface, such as user interface module 245 shown in FIG. 2.

In additional or alternative embodiments, obtaining the cytometry data includes processing the blood sample to obtain the cytometry data. For example, the blood sample may be processed using a cytometry platform (e.g., immune platform 114 in FIG. 1A, immune platform 270 in FIG. 2). For example, the cytometry platform may include any suitable flow cytometry platform. Flow cytometry may be performed using any suitable techniques such as, for example, the techniques described herein including in the section “Flow Cytometry.” Additionally, or alternatively, the cytometry platform may include any suitable mass cytometry platform. Mass cytometry may be performed using any suitable techniques such as, for example, the techniques described herein including in the section “Mass Cytometry.”

The cytometry data may include the cytometry data generated by a cytometry protocol, as well as information that can be inferred or determined from the cytometry data. The cytometry data may include information relating to a plurality of cells, for example, information relating to populations of immune cell types (e.g., PBMCs) of the subject. In some embodiments, the cytometry data comprises information relating to the presence, absence, and/or relative amounts of at least some (or all) of the cells of the plurality of cells. In some embodiments, the cytometry data comprises flow cytometry data. In some embodiments, the cytometry data comprises cytometry by time of flight (CyTOF) data.

At (optional) act 310, RNA expression data is obtained for the blood sample from the subject. For example, RNA expression data may be obtained for the blood sample as an alternative to obtaining cytometry data for the blood sample at act 308.

In some embodiments, the RNA expression data was previously obtained for the blood sample. Thus, in some embodiments, obtaining the RNA expression data may include accessing the data (e.g., from a memory, over a network, via a file being provided via an appropriate interface, etc.). For example, the RNA expression data may be obtained from a data store, such as data store(s) 280 shown in FIG. 2, and/or from user(s) (e.g., user(s) 290 shown in FIG. 2) providing a file including the segment data via an appropriate interface, such as user interface module 245 shown in FIG. 2. In additional or alternative embodiments, obtaining the RNA expression data includes processing the blood sample to obtain the RNA expression data. For example, the blood sample may be processed using a sequencing platform (e.g., sequencing platform 106 in FIG. 1A, sequencing platform 260 in FIG. 2). The origin, type, or preparation of the RNA expression data may include any of the embodiments described herein including at least with respect to FIG. 1A and with respect to the section “Sequencing Data.”

At act 312, a G2 score is determined using the cytometry data obtained at act 308 or the RNA expression data obtained at act 310. In some embodiments, determining a G2 score includes (a) determining cell composition percentages for cell types in the blood sample, (b) normalizing the cell composition percentages relative to a percentage of PBMCs (e.g., the total percentage of PBMCs of all types, or a sum of percentages of PBMCs of a plurality of types) in the blood sample, (c) normalizing the cell composition percentages relative to corresponding cell composition percentages in training data comprising a plurality of training samples, (d) determining an (unnormalized) G2 score for the blood sample using the normalized cell composition percentages and a G2 statistical model, and (e) (optionally) normalizing the (unnormalized) G2 score using G2 scores obtained for the training samples. Aspects of determining a G2 score for a subject using cell composition percentages are described herein including at least in the section “Immunoprofile Type Scores” and with respect to FIG. 5A.

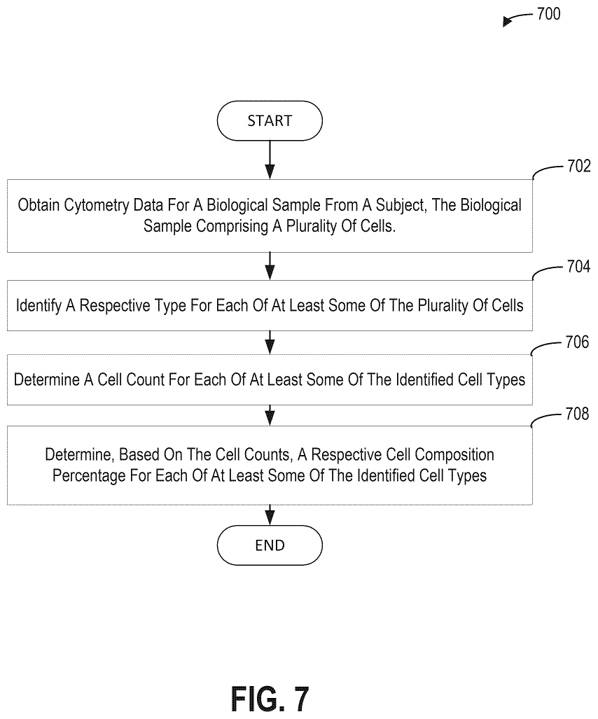

In some embodiments, cell composition percentages are determined using the cytometry data obtained at act 308 or the RNA expression data obtained at act 310. Examples of determining cell composition percentages are described herein including at least with respect to FIG. 1B, FIG. 7, and with respect to the section “Cell Composition Percentages.”

At act 314, a statistical model is used to predict, based on the selected MF profile type, the G2 score, and/or the PD-L1 expression, whether the subject will respond to an ICI therapy. The statistical model may include any suitable statistical model used to predict whether a subject will respond to an ICI therapy. A suitable statistical model may be any multivariate model that can be used to classify an observation comprising values for a plurality of predictive variables (e.g., MF profile type, G2 score, PD-L1 expression level, etc.) between two or more classes (e.g., classify a sample as responsive/non-responsive). For example, the statistical model may include a generalized linear model (e.g., a linear regression model, a logistic regression model, a probit regression model, etc.). It should be appreciated that, in some embodiments, the statistical model may not be a generalized linear model and may be a different type of statistical model such as, for example, a random forest regression model, a neural network, a support vector machine, a Gaussian mixture model, a hierarchical Bayesian model, and/or any other suitable statistical model, as aspects of the technology described herein are not limited to using generalized linear models for predicting therapeutic response. In some embodiments, the statistical model is a classifier trained to classify subjects between a responsive and a non-responsive class.

In some embodiments, the statistical model (e.g., a regression model) has a regression variable (also referred to as “predictor” or “predictive variable”) for the MF profile type (e.g., encoded MF profile type) selected for the tumor sample. In some embodiments, the statistical model includes a coefficient for the MF profile type. In some embodiments, the coefficient is estimated using (a) MF profile types determined for training tumor samples, (b) (optionally) values obtained for one or more other regression variables (including e.g., a G2 score), and (c) information indicating which of the training tumor samples were obtained from subjects who responded to the ICI therapy and/or which of the training tumor samples were obtained from subjects who were not responsive to the ICI therapy.

Additionally, or alternatively, in some embodiments, the statistical model has a regression variable for the G2 score determined for the blood sample. In some embodiments, the statistical model includes a coefficient for the G2 score. In some embodiments, the coefficient is estimated using (a) G2 scores determined for training blood samples, (b) (optionally) values obtained for one or more other regression variables, and (c) information indicating which of the training blood samples were obtained from subjects who responded to the ICI therapy and/or which of the training tumor samples were obtained from subjects who were not responsive to the ICI therapy.

Additionally, or alternatively, in some embodiments, the statistical model has a regression variable for the PD-L1 expression determined for the tumor sample. In some embodiments, the statistical model includes a coefficient for the PD-L1 expression. In some embodiments, the coefficient is estimated using (a) PD-L1 expression determined for training tumor samples, (b) (optionally) values obtained for one or more other regression variables, and (c) information indicating which of the training tumor samples were obtained from subjects who responded to the ICI therapy and/or which of the training tumor samples were obtained from subjects who were not responsive to the ICI therapy.

Table 1 shows example coefficients of regression variables in a statistical model. Examples of determining the example coefficients are described herein including at least in connection with the “Examples” sections.

TABLE 1
Example coefficients of regression variables
in a logistic regression model.

| Coefficient | Estimate |
|---|---|
| Intercept | −1.17972264 |
| MF Profile Type | 1.07614208 |
| G2 Score | 1.41367551 |
| PD-L1 Expression | 1.06043902 |

In some embodiments, the statistical model is regularized. For example, regularization techniques may be used when the statistical model includes more than one predictor. The statistical model may be regularized using any suitable regularization techniques such as, for example, L1 and/or L2 regularization.

In some embodiments, the output of the statistical model is indicative of whether the subject will respond to an ICI therapy. For example, the output may be a likelihood (e.g., a probability) that the subject will respond to an ICI therapy. Additionally, or alternatively, the output may be a binary value indicating whether or not the subject will respond to the ICI therapy. It should be appreciated, however, that the output may include any suitable output indicative of whether or not the subject will respond to the ICI therapy, as aspects of the technology described herein are not limited in this respect.

At (optional) act 316, the ICI therapy is recommended for the subject and/or the subject is selected for treatment with the ICI therapy. For example, if, at act 314, the subject is predicted to respond to the ICI therapy, the ICI therapy may be recommended for administration to the subject. For example, the recommendation may be in any suitable format such as, for example, in a report output to a user.

At (optional) act 318, the ICI therapy is administered to the subject. For example, the ICI therapy may be administered by a healthcare provider treating the subject. The ICI therapy may be administered according to embodiments described herein including with respect to the “Therapies” section.

FIG. 3B is a flowchart of an illustrative process 350 for predicting, using cell population data, whether a subject will respond to an ICI therapy, according to some embodiments of the technology described herein. One or more acts (e.g., all acts) of process 350 may be performed automatically by any suitable computing device(s). For example, the act(s) may be performed by a laptop computer, a desktop computer, one or more servers, in a cloud computing environment, computing device 1400 as described herein including with respect to FIG. 14, and/or in any other suitable way. Any feature described in the context of the methods described by reference to FIG. 3A are equally applicable to the methods described by reference to FIG. 3B unless context indicates otherwise.

At act 352, RNA expression data is obtained for a tumor sample from a subject. Aspects relating to RNA expression data and techniques for obtaining same are described herein including at least with respect to act 302 of process 300 shown in FIG. 3A.

At act 354, an MF profile type is selected for the tumor sample from among multiple MF profile types using the RNA expression data obtained at act 352. Aspects relating to MF profile types and techniques for selecting an MF profile type for a tumor sample are described herein including at least with respect to act 304 of process 300 shown in FIG. 3A.

At (optional) act 356, an expression of PD-L1 in the tumor sample is determined using the RNA expression data obtained at act 352. Aspects relating to PD-L1 expression and techniques for determining same are described herein including at least with respect to act 306 of process 300 shown in FIG. 3A.

At act 358, cell population data is obtained for a blood sample from the subject. In some embodiments, the cell population data was previously obtained for the blood sample. Thus, in some embodiments, obtaining the cell population data may include accessing the data (e.g., from a memory, over a network, via a file being provided via an appropriate interface, etc.). For example, the cell population data may be obtained from a data store (e.g., data store(s) 280 shown in FIG. 2), from a sequencing platform (e.g., sequencing platform 106 shown in FIG. 1A, sequencing platform 260 shown in FIG. 2, etc.), from an immune platform (e.g., immune platform 114 shown in FIG. 1A, immune platform 270 shown in FIG. 2, etc.) and/or from user(s) (e.g., user(s) 290 shown in FIG. 2) providing a file including the segment data via an appropriate interface (e.g., user interface module 245 shown in FIG. 2).

In additional or alternative embodiments, obtaining the cell population data includes processing the blood sample to obtain the cell population data. For example, the blood sample may be processed using an immune platform (e.g., immune platform 114 in FIG. 1A, immune platform 270 in FIG. 2, etc.) and/or a sequencing platform (e.g., sequencing platform 106 shown in FIG. 1A, sequencing platform 260 shown in FIG. 2, etc.).

The cell population data may include information relating to a plurality of cells, for example, information relating to populations of immune cell types (e.g., PBMCs) of the subject. In some embodiments, the cell population data comprises information relating to the presence, absence, and/or relative amounts of at least some (or all) of the cells of the plurality of cells. Aspects of cell population data are described herein including at least with respect to cell population data 116 shown in FIG. 1A and FIG. 1B.

At act 360, a G2 score is determined using the cell population data obtained at act 358. In some embodiments, determining a G2 score includes (a) determining cell composition percentages for cell types in the blood sample, (b) normalizing the cell composition percentages relative to a percentage of PBMCs (e.g., the total percentage of PBMCs of all types, or a sum of percentages of PBMCs of a plurality of types) in the blood sample, (c) normalizing the cell composition percentages relative to corresponding cell composition percentages in training data comprising a plurality of training samples, (d) determining an (unnormalized) G2 score for the blood sample using the normalized cell composition percentages and a G2 statistical model, and (c) (optionally) normalizing the (unnormalized) G2 score using G2 scores obtained for the training samples. Aspects of determining a G2 score for a subject using cell composition percentages are described herein including at least in the section “Immunoprofile Type Scores” and with respect to FIG. 5A.

In some embodiments, cell composition percentages are determined using the cell population data obtained at act 308. Examples of determining cell composition percentages are described herein including at least with respect to FIG. 1B, FIG. 7, and with respect to the section entitled “Cell Composition Percentages.”

At act 362, a statistical model is used to predict, based on the selected MF profile type, the G2 score, and/or the PD-L1 expression, whether the subject will respond to an ICI therapy. Aspects relating to statistical models and techniques for using a statistical model for predicting a subject's therapeutic response are described herein including at least with respect to act 314 of process 300 shown in FIG. 3A.

At (optional) act 364, the ICI therapy is recommended for the subject and/or the subject is selected for treatment with the ICI therapy. Aspects relating to techniques for recommending an ICI therapy for a subject are described herein including at least with respect to act 316 of process 300 shown in FIG. 3A.

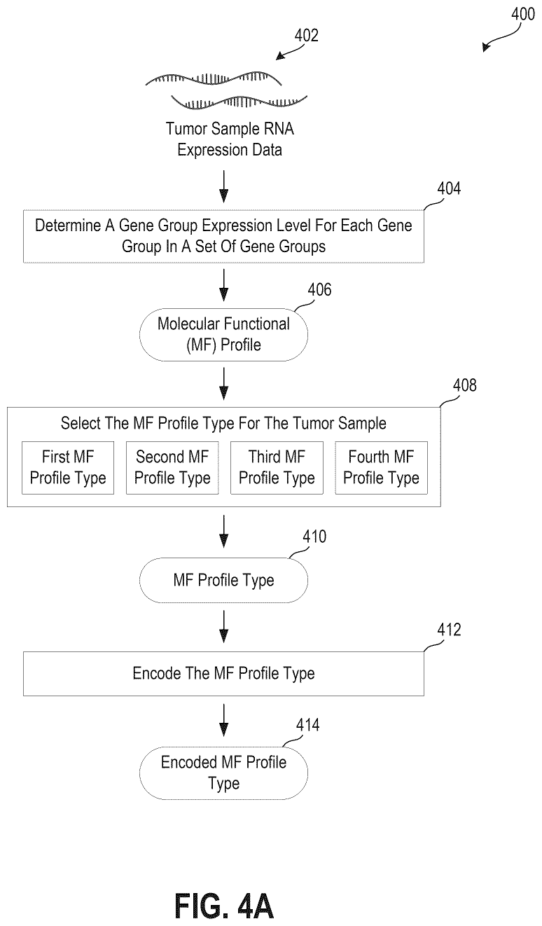

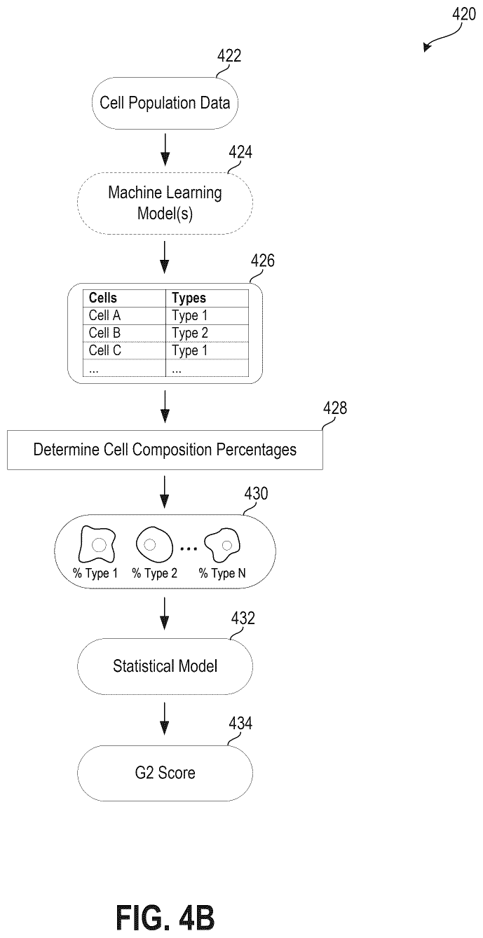

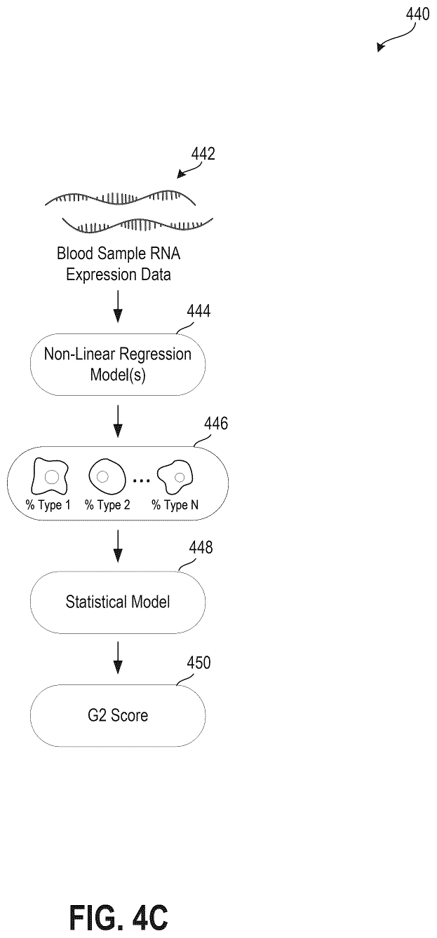

FIG. 4A is an illustrative example of selecting a molecular functional (MF) profile type for a subject, according to some embodiments of the technology described herein. Example 400 is an example implementation of act 304 of process 300.

In the example 400, RNA expression data 402 (e.g., tumor RNA expression data 108 in FIG. 1A and FIG. 1B) is processed to obtain an encoded MF profile type 414 for the tumor sample from which the RNA expression data 402 was obtained.

In the example, processing the RNA expression data 402 includes (a) at act 404, determining a gene group expression level for each gene group in a set of gene groups, (b) using the gene group expression levels to determine an MF profile 406 for the tumor sample, (c) at act 408, using the MF profile 406 to select the MF profile type 410 for the tumor sample, and (d) at act 412, encoding the MF profile type 410 to obtain the encoded MF profile type 414.

Example techniques for determining an MF profile for a tumor sample are described herein including at least with respect to the section “MF Profiles.”

In some embodiments the MF profile type is selected from among multiple MF profile types. For example, the MF profile type may be selected from among four MF profile types. For example, the first MF profile type may include an immune-enriched/fibrotic MF profile type, a second MF profile type may include an immune-enriched/non-fibrotic MF profile type, a third MF profile type may include a fibrotic MF profile type (e.g., fibrotic/non-immune-enriched), and a fourth MF profile type may include an immune desert MF profile type (e.g., non-fibrotic/non-immune-enriched). Aspects of MF profile types are described herein including at least in the section “MF Profile Types.” Example techniques for selecting an MF profile type for a tumor sample are described herein including at least with respect to FIG. 8A and FIG. 8B, and with respect to the section “Selecting MF Profile Types.”

In some embodiments, encoding the MF profile type at act 412 may include assigning a numerical value to the MF profile type 410 or encoding the MF profile type 410 using any other suitable encoding techniques, as aspects of the technology described herein are not limited in this respect. For example, encoding the MF profile type 410 may include assigning a first value to the MF profile type 410 when the MF profile type 410 is of the first MF profile type or the second MF profile type and assigning a second, different value to the MF profile type 410 when the MF profile type 410 is of the third MF profile type or the fourth MF profile type. For example, a 1 may be assigned when the MF profile type 410 is of the first or second MF profile type and a 0 may be assigned when the MF profile type 410 is of the third or fourth MF profile type.

FIG. 4B is an illustrative example of determining a G2 score for a blood sample using cell population data 422, according to some embodiments of the technology described herein. Example 420 is an example implementation of act 312 of process 300. For example, the cell population data 422 may include cytometry data and/or hematology data that lists a cell type for each cell detected in the sample.

As shown in FIG. 4B, example implementation 420 includes processing cell population data 422 obtained for a blood sample from a subject to obtain a G2 score 434 for the blood sample.

In some embodiments, the processing includes: (a) (optionally) applying machine learning model(s) 424 to the cell population data 422 to determine cell types 426 for cells in the blood sample, (b) at act 428, determining cell composition percentages 430 using the determined cell types 426, and (d) processing the cell composition percentages 430 using a statistical model 432 to obtain the G2 score.

Example techniques for determining types for cells in a blood sample and using the types to determine cell composition percentages are described herein including at least with respect to FIG. 7 and in the section “Cell Composition Percentages.”

Example techniques for processing cell composition percentages using a statistical model to obtain a G2 score are described herein including at least with respect to FIG. 5A and in the section “Immunoprofile Type Scores.”

FIG. 4C is an illustrative example of determining a G2 score for a blood sample using RNA expression data, according to some embodiments of the technology described herein. Example 440 is an example implementation of act 312 of process 300.

As shown in FIG. 4C, example implementation 440 includes processing RNA expression data obtained for a blood sample from a subject to obtain a G2 score 450 for the blood sample.

In some embodiments, the processing includes: (a) applying non-linear regression model(s) 444 to the RNA expression data 442 to determine cell composition percentages 446 and (b) processing the cell composition percentages 446 using a statistical model 432 to obtain the G2 score.

Example techniques for cell composition percentages using RNA expression data are described herein including at least with respect to the section “Cell Composition Percentages.”

Example techniques for processing cell composition percentages using a statistical model to obtain a G2 score are described herein including at least with respect to FIG. 5A and in the section “Immunoprofile Type Scores.”

### Immunoprofile Type Scores

Aspects of the disclosure relate to determining a G2 score for a blood sample by processing cell population data. For example, the cell population data may be processed to determine cell composition percentage for at least some cell types in the biological sample, and the cell composition percentages may be used to determine the G2 score. Example techniques for determining cell composition percentages are described herein including at least in the section “Cell Composition Percentages.” In some embodiments, the G2 score is a metric that separates samples of the G2 immunoprofile type from samples of non-G2 immunoprofile types (e.g., G1, G3, G4, and G5). Example aspects of immunoprofile types and selecting an immunoprofile type for a subject are described in International Application No. PCT/US2023/080339, published as International Publication No. WO2024/108156 on May 5, 2023, the entire contents of which are incorporated by reference herein.

FIG. 5A is a flowchart of an illustrative process 500 for determining a G2 score for a blood sample, according to some embodiments of the technology described herein. Process 500 may be used to implement act 312 of process 300 shown in FIG. 3A and/or act 360 of process 350 shown in FIG. 3B. Process 500 may be performed in part or in full by a laptop computer, a desktop computer, one or more servers, in a cloud computing environment, computing device as described herein with respect to FIG. 14 or using any other suitable computing device(s), as aspects of the technology described herein are not limited in this respect.

Process 500 begins at act 502 for obtaining cell composition percentages for types of cells in the blood sample. In some embodiments, act 502 may be performed in any suitable way as described herein. For example, cell composition percentages may be obtained by processing cell population data obtained for the blood sample. Example techniques for determining cell composition percentages are described herein including at least in the section “Cell Composition Percentages.” In some embodiments, a cell composition percentage may be obtained for peripheral blood mononuclear cells (PBMCs) in the blood sample (e.g., the total percentage of PBMCs of all types, or a sum of percentages of PBMCs of a plurality of types). In some embodiments, a cell composition percentage may be obtained for each of a plurality of immune cell types (e.g. a plurality of types of peripheral blood mononuclear cells) in the blood sample. Additionally, or alternatively, in some embodiments, cell composition percentages may be obtained for at least some (e.g., all) of the cell types listed in Table 2, the cell types listed in Table 3, and/or the cell types listed in Table 4. For example, if the cell composition percentages are determined by processing cytometry data for the blood sample, the cell composition percentages may be obtained for one or more or all of the types listed in Table 2. Additionally, or alternatively, if the cell composition percentages are determined by processing RNA expression data for the blood sample, the cell composition percentages may be obtained for one or more or all of the cell types listed in Table 3. Additionally, or alternatively, if the cell composition percentages are determined by processing the blood sample using a hematology analyzer, the cell composition percentages may be obtained for one or more or all of the cell types listed in Table 4.

Next, at act 504, at least some of the cell composition percentages obtained at act 502 are normalized relative to the cell composition percentage of peripheral blood mononuclear cells (PBMCs) in the blood sample (e.g., the total percentage of PBMCs of all types, or a sum of percentages of PBMCs of a plurality of types). For example, cell composition percentages for cell types listed in Table 2, Table 3, and/or Table 4 may be normalized relative to the cell composition percentage of PBMCs (e.g., the total percentage of PBMCs of all types, or a sum of percentages of PBMCs of a plurality of types). Any suitable normalization techniques may be performed relative to the cell composition percentage of PBMCs. For example, the normalizing may include dividing the cell composition percentages by the cell composition percentage of PBMCs (e.g., the total percentage of PBMCs of all types, or a sum of percentages of PBMCs of a plurality of types).

At act 506, the normalized cell composition percentages obtained at act 504 may be normalized relative to cell composition percentages for cell types in training data comprising a plurality of training samples. The training samples may be obtained or may have been previously obtained from one or more healthy subjects (e.g., subjects who do not have, are not suspected of having and/or are not at risk of having cancer) and/or one or more subjects with solid tumors. In some embodiments, the training data includes an indication of an immunoprofile type for the training sample.

In some embodiments, the indication of the immunoprofile type may include an indication of whether the training sample has been classified as G1 type, G2 type, G3 type, G4 type, or G5 type. In some embodiments, the indication includes any suitable indication, as aspects of the technology described herein are not limited in this respect. For example, the indication may be encoded by assigning a value of 1 to samples classified as G2 type and by assigning a value of 0 to samples classified as non-G2 types. Example techniques for determining an immunoprofile type for a subject are described in the section “Selecting Immunoprofile Types.”

In some embodiments, the cell composition percentages in the training data includes cell composition percentages of PBMCs in the training samples and/or cell composition percentages for cell types listed in Table 2, Table 3, and/or Table 4 in the training samples. In some embodiments, the cell composition percentages in the training data are normalized. For example, the cell composition percentages (e.g., cell composition percentages for cell types listed in Table 2, Table 3, and/or Table 4) obtained for a training sample may be normalized relative to the cell composition percentage of PBMCs in the training sample (e.g., the total percentage of PBMCs of all types, or a sum of percentages of PBMCs of a plurality of types).

In some embodiments, the training cell composition percentages may be obtained using any suitable techniques, as aspects of the technology described herein are not limited in this respect. For example, in some embodiments, the cell composition percentages are obtained from a data store (e.g., a public data store). In some embodiments, the cell composition percentages are obtained for the blood samples by processing cell population data and/or RNA expression data obtained for the blood samples. For example, the cell population data and/or RNA expression data may be obtained from a data store (e.g., a public data store), by processing blood samples from one or more subjects, or obtained in any other suitable manner, as aspects of the technology described herein are not limited in this respect.

In some embodiments, the normalizing is performed using any suitable normalization technique, as aspects of the technology described herein is not limited in this respect. For example, in some embodiments, the normalizing is performed using quantiles of the distribution of cell composition percentages (e.g., normalized cell composition percentages) in the training data. For example, the normalizing may be performed using at least two quantiles of the distribution of cell composition percentages in the training data. The quantile(s) may be any suitable quantile(s) as aspects of the technology described herein are not limited in this respect. For example, a first quantile (e.g., q1) may be the 0.01 quantile, the 0.02 quantile, the 0.03 quantile, the 0.04 quantile, the 0.05 quantile, any quantile between the 0.01 quantile and the 0.1 quantile, or any other suitable quantile as aspects of the technology described herein are not limited in this respect. Additionally, or alternatively, the second quantile (e.g., q2) may be the 0.90 quantile, the 0.95 quantile, the 0.96 quantile, the 0.97 quantile, the 0.98 quantile, the 0.99 quantile, any quantile between the 0.90 quantile and the 0.99 quantile, or any other suitable quantile as aspects of the technology described herein are not limited in this respect. As one nonlimiting example, the normalizing may be performed using the 0.02 quantile and the 0.98 quantile of the training data.

Equation 1 is an example equation for normalizing a cell composition percentage (CCP) to obtain a normalized cell composition percentage (CCP_{N}). However, it should be appreciated that the cell composition percentages may be normalized according to any other suitable techniques, as aspects of the technology described herein are not limited in this respect.

$$C C {P}_{N} = ( C C P - q 1 ) / ( q 2 - q 1 ) \qquad ( Equation 1 )$$

In some embodiments, the normalized cell composition percentages may be adjusted. For example, normalized cell composition percentages greater than a predetermined value (e.g., one) may be replaced with a value of one. Additionally, or alternatively, normalized cell composition percentages less than a predetermined value (e.g., zero) may be replaced with a value of zero.

At act 508, an unnormalized G2 score is determined for the biological sample using the normalized cell composition percentages and a G2 score statistical model. In some embodiments, this includes determining a combination (e.g., linear or non-linear) of the normalized cell composition percentages. In some embodiments, determining the combination of normalized cell composition percentages includes using previously determined coefficients to determine a weighted sum of the normalized cell composition percentages, as described herein. The G2 score statistical model may include any suitable statistical model. A suitable statistical model may be any multivariate model that can be used to classify an observation comprising values for a plurality of cell composition percentages. For example, the statistical model may be a generalized linear model (e.g., a linear regression model, a logistic regression model, a probit regression model, an Elastic Net regression model, etc.). It should be appreciated that, in some embodiments, the statistical model may not be a generalized linear model and may be a different type of statistical model such as, for example, a random forest regression model, a neural network, a support vector machine, a Gaussian mixture model, a hierarchical Bayesian model, and/or any other suitable statistical model, as aspects of the technology described herein are not limited to using generalized linear models for determining the unnormalized G2 score.

In some embodiments, the statistical model is trained by determining coefficients for the normalized cell composition percentages, and using the coefficients to determine a weighted sum of the normalized cell composition percentages. For example, coefficients may be estimated based on training data (e.g., the training set of cell composition percentages). Example coefficients are listed for cell types in Table 2, Table 3, and Table 4. In some embodiments, the training data includes, for each training sample, the cell composition percentages and a known immunoprofile type. In some embodiments, indications of known immunoprofile types (e.g., encoded as 0 and 1) are used as target values for the regression. In some embodiments, the coefficients are estimated by performing a regression analysis on the training data.

At act 512, the unnormalized G2 scores (e.g., for the blood sample and/or for the training samples) may optionally be normalized. For example, the unnormalized G2 scores may be normalized to range of values having any suitable upper bound and any suitable lower bound, as aspects of the technology described herein are not limited in this respect. For example, the lower bound may be a value between 0.01 and 0.50, between 0.02 and 0.45, between 0.03 and 0.40, between 0.04 and 0.35, between 0.05 and 0.30, between 0.06 and 0.25, between 0.07 and 0.20, between 0.08 and 0.15, or a value in any other suitable range as aspects of the technology described herein are not limited in this respect. Additionally, or alternatively, the upper bound may be a value between 5 and 15, between 6 and 14, between 7 and 13, between 8 and 12, between 9 and 11, or a value in any other suitable range of values as aspects of the technology described herein are not limited in this respect.

In some embodiments, the normalizing may be performed using any suitable normalization technique, as aspects of the technology described herein are not limited in this respect. In some embodiments, the normalizing is performed using quantiles of the G2 scores determined for training samples. For example, the normalizing may be performed using at least two quantiles of the distribution of G2 scores determined for the training samples. The quantile(s) may be any suitable quantile(s) as aspects of the technology described herein are not limited in this respect. For example, a first quantile (e.g., qp1) may be the 0.01 quantile, the 0.02 quantile, the 0.03 quantile, the 0.04 quantile, the 0.05 quantile, any quantile between the 0.01 quantile and the 0.1 quantile, or any other suitable quantile as aspects of the technology described herein are not limited in this respect. Additionally, or alternatively, the second quantile (e.g., qp2) may be the 0.90 quantile, the 0.95 quantile, the 0.96 quantile, the 0.97 quantile, the 0.98 quantile, the 0.99 quantile, any quantile between the 0.90 quantile and the 0.99 quantile, or any other suitable quantile as aspects of the technology described herein are not limited in this respect. As one nonlimiting example, the normalizing may be performed using the 0.01 quantile and the 0.99 quantile of the distribution of G2 scores determined for the training samples.

Equation 2 is an example equation for normalizing a G2 score for a blood sample to obtain a normalized G2 score (G2N). However, it should be appreciated that the cell composition percentages may be normalized according to any other suitable techniques, as aspects of the technology described herein are not limited in this respect.

$${G}_{2} = 9.9 * \frac{G 2 - q p 1}{q p 2 - q p 1} + . 1 \qquad ( Equation 2 )$$

FIG. 5B and FIG. 5C are example plots showing the relationship between immunoprofile types and G2 score, according to some embodiments of the technology described herein. As shown, the points in the cluster associated with the Primed (G2) immunotype correspond to the relatively low G2 scores. Points in clusters associated with the non-G2 immunotypes correspond to relatively low G2 scores.

TABLE 2
Example cell types and statistical model coefficients.

| Cell Type | Example Coefficient |
|---|---|
| Mature NK cells | −0.009719097277380727 |
| Immature NK cells | −0.0023116346621594383 |
| Non-classical Monocytes | −0.007650384996208602 |
| TIGIT+ PD1+ CD8 T cells | −0.03130695892542463 |
| gdT Vdelta2+ | 0.01046139624699525 |
| Naïve B cells | 0 |
| CD8 Memory T cells | 0 |
| Classical Monocytes | 0 |
| NKT cells | 0 |
| CD4 TEMRA | 0 |
| CD8 T cells | −0.20138774519045705 |
| CD4 Memory T helpers | 0.3998676216051481 |
| CD4 Tregs | 0.1681964038792675 |
| Class-switched Memory | 0.13866494404450544 |
| HLA-DR-low Monocytes | −0.0002529151631122323 |
| Plasmacytoid Dendritic cells | −0.12806129480800374 |
| CD4 T cells | 0.19914283540910158 |
| Dendritic cells | −0.16720419027738742 |
| Non-switched Memory IgM B cells | 0.021758080813785313 |
| CD8 CD45RA− CD27+ T cells | 0.03689773677262804 |
| CD8 CD45RA+ CD27+ T cells | −0.1917343109119336 |
| CD4 CD45RA− CD27+ T cells | 0.5660870754603151 |
| CD4 CD45RA+ CD27+ T cells | 0 |

TABLE 3
Example cell types and statistical model coefficients.

| Cell Type | Example Coefficient |
|---|---|
| B Cells | −0.05547850046595542 |
| CD4 T Cells | 0.15628774259744674 |
| CD8 T Cells | −0.11058775887422302 |
| CD8 T cells PD1 high | −0.10708606252783345 |
| CD8 T cells PD1 low: | 0.001252328540792983 |
| CDC | 0.0035701123628065876 |
| Central memory T helpers | 0.20624666396662497 |
| Class switched memory B cells | 0.12106827631578211 |
| Classical monocytes | 0.002690031847132537 |
| Cytotoxic NK cells | −0.09246279632059323 |
| Dendritic cells | −0.06503178249631833 |
| Effector memory T helpers | 0.03814117135520952 |
| Lymphoid cells | 0.004417263857474982 |
| Mature B cells | 0.07603476987375032 |
| Memory CD8 T cells | −0.07755642983727865 |
| Memory T cells | 0.07799955954957681 |
| Monocytes | 0.0033244610927536567 |
| NK cells | −0.09784852555248195 |
| Naïve B cells | −0.14190736148874944 |
| Naïve CD8 T cells | −0.13416892299083286 |
| Naïve T cells | −0.014163118651622803 |
| Naïve T helpers | 0.02617619417785783 |
| Non classical monocytes | −0.029064245425104968 |
| Non switched memory B cells | 0.030616073377453853 |
| PDC | −0.1654653359798737 |
| Regulatory NK cells | 0.040678540606709945 |
| Secreting B cells | 0.02975893444616884 |
| T cells | 0.07672998064534692 |
| Th17 cells | −0.003035691929079774 |
| Th1 cells | 0.2868243955820828 |
| Th2 cells | 0.2153405613683452 |
| Transitional memory T helpers | 0.15336244500668553 |
| Tregs | 0.2116235378024221 |

TABLE 4
Example cell types and statistical model coefficients.

| Cell Type | Example Coefficient |
|---|---|
| Basophils | −0.005947832140532374 |
| Eosinophils | −0.008987081067200002 |
| Lymphocytes | 0.02836110554103789 |
| Monocytes | −0.027941393926589998 |
| Neutrophils | 0.04903350216960409 |

### Immunoprofile Types

In some embodiments, immunoprofile types comprise a Naive type (G1), a Primed type (G2), a Progressive type (G3), a Chronic type (G4), and a Suppressive type (G5). The immunoprofile types (also referred to as PBMC immunoprofile types) described herein may be described by qualitative characteristics, for example by different cell composition percentages for different cell types. In some embodiments, a high cell composition percentage refers to higher cell composition percentage of the same cell type in the subject being analyzed compared to a different subject. In some embodiments, a low cell composition percentage refers to lower cell composition percentage of the same cell type in the subject being analyzed compared to a different subject. In some embodiments, a “high” signal refers to a cell composition percentage that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more increased relative to the cell composition percentage of the same cell type in a different subject. In some embodiments, a “low” signal refers to a cell composition percentage that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more decreased relative to the cell composition percentage of the same cell type in a different subject.

In some embodiments, the Suppressive PMBC immunoprofile type (G5) is characterized by an increased number of myeloid cell populations, including classical monocytes and neutrophils, relative to the other PMBC immunoprofile types.

In some embodiments, the Chronic PMBC immunoprofile type (G4) is characterized by an increased number of CD8 memory and effector cells as well as the NKT cell population, relative to the other PMBC immunoprofile types.

In some embodiments, the Progressive cell memory PMBC immunoprofile type (G3) is characterized by an increased number of CD4 and CD8 memory cells, and high increase in CD8 transitional memory cells, relative to the other PMBC immunoprofile types.

In some embodiments, the Primed PMBC immunoprofile type (G2) is characterized by an increased number of T-helper memory cells, including CD4 central memory, relative to the other PMBC immunoprofile types.

In some embodiments, the Naive PMBC immunoprofile type (G1) is characterized by an increased number of naive CD4, CD8 and B cells, relative to the other PMBC immunoprofile types.

In some embodiments, the immunoprofile types can also be described statistically. For example, each immunoprofile type may correspond to a respective cluster of PBMC signatures obtained for a plurality of training samples, and thus may be described in terms of the PBMC signature clusters. Tables 11-16 describe example PBMC signature clusters. Example aspects of immunoprofile types and selecting an immunoprofile type for a subject are described in International Application No. PCT/US2023/080339, published as International Publication No. WO2024/108156 on May 5, 2023, the entire contents of which are incorporated by reference herein.

### Selecting Immunoprofile Types

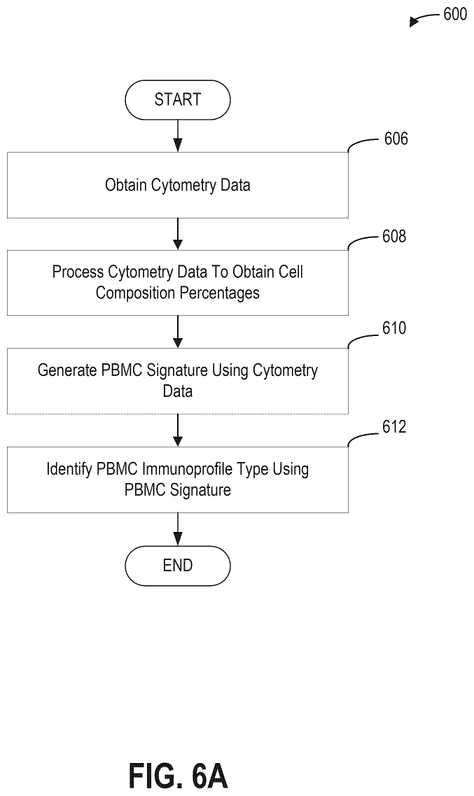

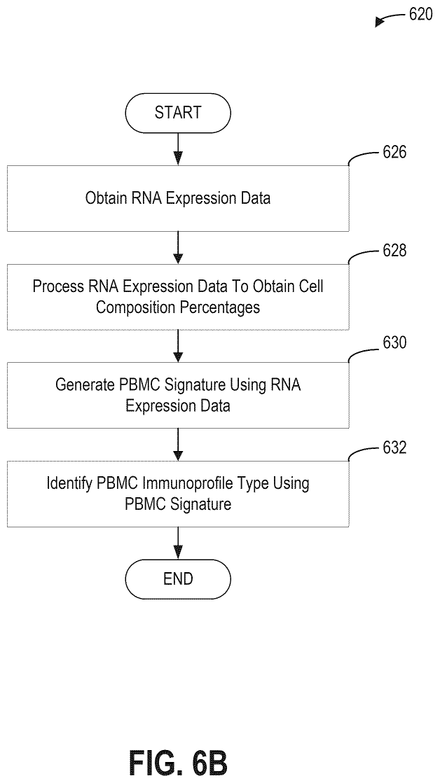

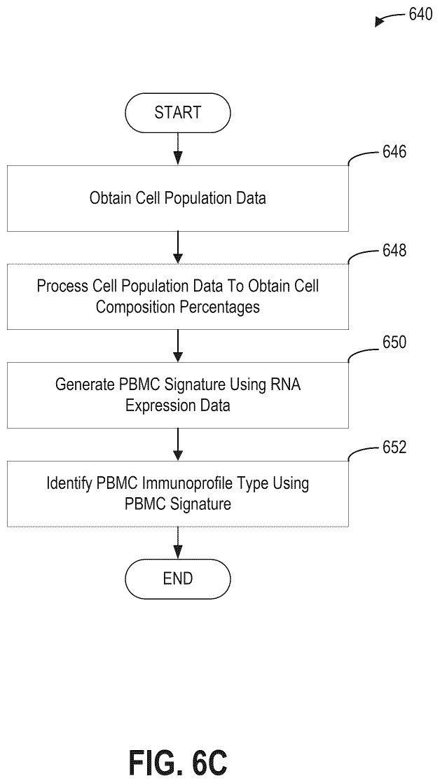

FIG. 6A depicts an illustrative process 600 for determining a determining a peripheral blood mononuclear cells (PBMC) immunoprofile type of a subject. In some embodiments, the subject may include any of the embodiments described herein including with respect to the “Subjects” section.

At act, 606, cytometry data is obtained for a biological sample (e.g., a blood sample) obtained (e.g., previously obtained) from the subject. The cytometry data may comprise information relating to a plurality of cells, for example, information relating to populations of immune cell types (e.g., PBMCs) of the subject. In some embodiments, the cytometry data comprises information relating to the presence, absence, and/or relative amounts of at least some (or all) of the cells of the plurality of cells, for example some or all of the cell types listed in Table 5, Table 6, and/or Table 7. In some embodiments, the cytometry data comprises flow cytometry data. In some embodiments, the cytometry data comprises cytometry by time of flight (CyTOF) data. In some embodiments, the cytometry data comprises spectral cytometry data.

In some embodiments, the cell population data comprises information relating to the presence, absence, and/or relative amounts for between 2 and 36 cell types listed in Table 5, Table 6, and/or Table 7. In some embodiments, the cytometry data comprises information relating to the presence, absence, and/or relative amounts for between 3 and 8, 5 and 12, 10 and 20, 15 and 25, 18 and 34 or 18 and 36 cell types listed in Table 5, Table 6, and/or Table 7. In some embodiments, the cytometry data comprises information relating to the presence, absence, and/or relative amounts for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, the cytometry data comprises information relating to the presence, absence, and/or relative amounts for additional cell types that are not listed in Table 5, Table 6, and/or Table 7.

Next, process 600 proceeds to act 608, processing the cytometry data to obtain cell composition percentages. In some embodiments, the cytometry data is processed to obtain cell composition percentages for at least some cell types of a plurality of cell types listed in Table 5, Table 6, and/or Table 7. In some embodiments, the cytometry data is processed to obtain cell composition percentages for between 2 and 36 cell types listed in Table 5, Table 6, and/or Table 7. In some embodiments, the cytometry data is processed to obtain cell composition percentages for between 2 and 34 cell types listed in Table 5. In some embodiments, the cell population data is processed to obtain cell composition percentages for between 3 and 8, 5 and 12, 10 and 20, 15 and 25, 18 and 34 or 18 and 36 cell types listed in Table 5, Table 6, and/or Table 7. In some embodiments, the cytometry data is processed to obtain cell composition percentages for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, the cytometry data is processed to obtain cell composition percentages for additional cell types that are not listed in Table 5, Table 6, and/or Table 7. Methods of processing cytometry data to obtain cell composition percentages are further described herein including at least with respect to the section entitled “Cell Composition Percentages”.

After cell composition percentages have been obtained from the cytometry data in act 608, process 600 proceeds to act 610, generating a PBMC signature using the cytometry data. In some embodiments, a PBMC signature comprises cell composition percentages for at least some of the cell types listed in Table 5, Table 6, and/or Table 7. In some embodiments, a PBMC signature comprises cell composition percentages for between 2 and 36 cell types listed in Table 5, Table 6, and/or Table 7. In some embodiments, a PBMC signature comprises cell composition percentages for between 3 and 8, 5 and 12, 10 and 20, 15 and 25, 18 and 34 or 18 and 36 cell types listed in Table 5, Table 6, and/or Table 7. In some embodiments, a PBMC signature comprises cell composition percentages for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, a PBMC signature comprises cell composition percentages for additional cell types that are not listed in Table 5, Table 6, and/or Table 7. In some embodiments, the PBMC signature is outputted as a vector comprising the cell composition percentages.

Next, process 600 proceeds to act 612, where a PBMC immunoprofile type is identified for the subject using the PBMC signature generated at act 610. This may be done in any suitable way. For example, in some embodiments, each of the possible PBMC immunoprofile types is associated with a respective plurality of PBMC signature clusters. In such embodiments, a PBMC immunoprofile type for the subject may be identified by associating the PBMC signature of the subject with a particular one of the plurality of PBMC signature clusters (e.g., the type identified may be the type associated with the PBMC signature cluster to which the PBMC signature of the subject is closest according to a distance measure or any suitable measure of distance or similarity); and identifying the PBMC immunoprofile type for the subject as the PBMC immunoprofile type corresponding to the particular one of the plurality of PBMC signature clusters to which the PBMC signature of the subject is associated. Examples of PBMC immunoprofile types are described herein.

As described above, a subject's PBMC immunoprofile type is identified at act 612. In some embodiments, the PBMC immunoprofile type of a subject is identified to be one of the following PBMC immunoprofile types: Naive type (G1), Primed type (G2), Progressive type (G3), Chronic type (G4), or Suppressive type (G5). In some embodiments, process 600 ends once act 612 is complete.

FIG. 6B depicts an illustrative process 620 for determining a peripheral blood mononuclear cells (PBMC) immunoprofile type of a subject having, suspected of having, or at risk of having cancer. In some embodiments, the subject may include any of the embodiments described herein including with respect to the “Subjects” section.

At act 626, RNA expression data is obtained for the subject. The RNA expression data, in some embodiments, comprises RNA expression levels for genes expressed by a plurality of cells, for example, a plurality of immune cell types (e.g., PBMCs), of the subject. In some embodiments, the RNA expression data comprises information (e.g., RNA expression levels) relating to the presence, absence, and/or relative amounts of at least some (or all) of the cells of the plurality of cells, for example some or all of the cell types listed in Table 5, Table 6, and/or Table 7.

In some embodiments, the RNA expression data comprises RNA expression levels of genes associated with between 2 and 36 cell types listed in Table 5, Table 6, and/or Table 7. In some embodiments, a gene that is associated with a cell type is a gene that is differentially expressed in the cell type compared to its expression in the other cell types. In some embodiments, the RNA expression data comprises RNA expression levels of genes associated with between 3 and 8, 5 and 12, 10 and 20, 15 and 25, 18 and 34 or 18 and 36 cell types listed in Table 5, Table 6, and/or Table 7. In some embodiments, the RNA expression data comprises RNA expression levels of genes associated with at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, the RNA expression data comprises RNA expression levels of genes associated with additional cell types that are not listed in Table 5, Table 6, and/or Table 7.

Next, process 620 proceeds to act 628, processing the RNA expression data to obtain cell composition percentages. In some embodiments, the RNA expression data is processed to obtain cell composition percentages for at least some cell types of a plurality of cell types listed in Table 5, Table 6, and/or Table 7. In some embodiments, the RNA expression data is processed to obtain cell composition percentages for between 2 and 36 cell types listed in Table 5, Table 6, and/or Table 7. In some embodiments, the RNA expression data is processed to obtain cell composition percentages for between 3 and 8, 5 and 12, 10 and 20, 15 and 25, 18 and 34 or 18 and 36 cell types listed in Table 5, Table 6, and/or Table 7. In some embodiments, the RNA expression data is processed to obtain cell composition percentages for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, the RNA expression data is processed to obtain cell composition percentages for additional cell types that are not listed in Table 5, Table 6, and/or Table 7.

In some embodiments, act 628 comprises processing the RNA expression levels using a cell deconvolution technique (e.g., a computational technique used to estimate the proportions of different cell types in samples) to determine the cell composition percentages for at least some (or all) cell types of a plurality of cell types listed in Table 5, Table 6, and/or Table 7. Methods of processing cytometry data to obtain cell composition percentages are further described herein including at least with respect to the section entitled “Cell Composition Percentages”.

After cell composition percentages have been obtained from the RNA expression data in act 628, process 620 proceeds to act 230, generating a PBMC signature using the RNA expression data. In some embodiments, a PBMC signature comprises cell composition percentages for at least some of the cell types listed in Table 5, Table 6, and/or Table 7. In some embodiments, a PBMC signature comprises cell composition percentages for between 2 and 36 cell types listed in Table 5, Table 6, and/or Table 7 or for between 2 and 34 cell types listed in Table 6. In some embodiments, a PBMC signature comprises cell composition percentages for between 3 and 8, 5 and 12, 10 and 20, 15 and 25, 18 and 34 or 18 and 36 cell types listed in Table 5, Table 6, and/or Table 7. In some embodiments, a PBMC signature comprises cell composition percentages for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, a PBMC signature comprises cell composition percentages for additional cell types that are not listed in Table 5, Table 6, and/or Table 7. In some embodiments, the PBMC signature is outputted as a vector comprising the cell composition percentages.

Next, process 620 proceeds to act 632, where a PBMC immunoprofile type is identified for the subject using the PBMC signature generated at act 610. This may be done in any suitable way. For example, in some embodiments, each of the possible PBMC immunoprofile types is associated with a respective plurality of PBMC signature clusters. In such embodiments, a PBMC immunoprofile type for the subject may be identified by associating the PBMC signature of the subject with a particular one of the plurality of PBMC signature clusters (e.g., the type identified may be the type associated with the PBMC signature cluster to which the PBMC signature of the subject is closest according to a distance measure or any suitable measure of distance or similarity); and identifying the PBMC immunoprofile type for the subject as the PBMC immunoprofile type corresponding to the particular one of the plurality of PBMC signature clusters to which the PBMC signature of the subject is associated. Examples of PBMC immunoprofile types are described herein.

As described above, a subject's PBMC immunoprofile type is identified at act 632. In some embodiments, the PBMC immunoprofile type of a subject is identified to be one of the following PBMC immunoprofile types: Naïve (G1) type, Primed (G2) type, Progressive (G3) type, Chronic (G4) type, or Suppressive (G5) type.

FIG. 6C depicts an illustrative process 640 for determining a determining a peripheral blood mononuclear cells (PBMC) immunoprofile type of a subject using cell population data. In some embodiments, the subject may include any of the embodiments described herein including with respect to the “Subjects” section.

At act, 646, cell population data is obtained for a biological sample (e.g., a blood sample) obtained (e.g., previously obtained) from the subject. The cell population data may comprise information relating to a plurality of cells, for example, information relating to populations of immune cell types (e.g., PBMCs) of the subject. In some embodiments, the cell population data comprises information relating to the presence, absence, and/or relative amounts of at least some (or all) of the cells of the plurality of cells, for example some or all of the cell types listed in Table 5, Table 6, and/or Table 7. In some embodiments, the cell population data comprises cell population data 116 described herein including at least with respect to FIG. 1A and FIG. 1B.

In some embodiments, the cell population data comprises information relating to the presence, absence, and/or relative amounts for between 2 and 36 cell types listed in Table 5, Table 6, and/or Table 7. In some embodiments, the cell population data comprises information relating to the presence, absence, and/or relative amounts for between 3 and 8, 5 and 12, 10 and 20, 15 and 25, 18 and 34 or 18 and 36 cell types listed in Table 5, Table 6, and/or Table 7. In some embodiments, the cell population data comprises information relating to the presence, absence, and/or relative amounts for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, the cell population data comprises information relating to the presence, absence, and/or relative amounts for additional cell types that are not listed in Table 5, Table 6, and/or Table 7.

Next, process 640 proceeds to act 648, processing the cell population data to obtain cell composition percentages. In some embodiments, the cell population data is processed to obtain cell composition percentages for at least some cell types of a plurality of cell types listed in Table 5, Table 6, and/or Table 7. In some embodiments, the cell population data is processed to obtain cell composition percentages for between 2 and 36 cell types listed in Table 5, Table 6, and/or Table 7. In some embodiments, the cell population data is processed to obtain cell composition percentages for between 2 and 34 cell types listed in Table 5. In some embodiments, the cell population data is processed to obtain cell composition percentages for between 3 and 8, 5 and 12, 10 and 20, 15 and 25, 18 and 34 or 18 and 36 cell types listed in Table 5, Table 6, and/or Table 7. In some embodiments, the cell population data is processed to obtain cell composition percentages for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, the cell population data is processed to obtain cell composition percentages for additional cell types that are not listed in Table 5, Table 6, and/or Table 7. Methods of processing cell population data to obtain cell composition percentages are further described herein including at least with respect to the section entitled “Cell Composition Percentages”.

After cell composition percentages have been obtained from the cell population data in act 648, process 640 proceeds to act 650, generating a PBMC signature using the cell population data. In some embodiments, a PBMC signature comprises cell composition percentages for at least some of the cell types listed in Table 5, Table 6, and/or Table 7. In some embodiments, a PBMC signature comprises cell composition percentages for between 2 and 36 cell types listed in Table 5, Table 6, and/or Table 7. In some embodiments, a PBMC signature comprises cell composition percentages for between 3 and 8, 5 and 12, 10 and 20, 15 and 25, 18 and 34 or 18 and 36 cell types listed in Table 5, Table 6, and/or Table 7. In some embodiments, a PBMC signature comprises cell composition percentages for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, or 36 cell types listed in Table 5, Table 6, and/or Table 7. In some embodiments, a PBMC signature comprises cell composition percentages for additional cell types that are not listed in Table 5, Table 6, and/or Table 7. In some embodiments, the PBMC signature is outputted as a vector comprising the cell composition percentages.

Next, process 640 proceeds to act 652, where a PBMC immunoprofile type is identified for the subject using the PBMC signature generated at act 610. This may be done in any suitable way. For example, in some embodiments, each of the possible PBMC immunoprofile types is associated with a respective plurality of PBMC signature clusters. In such embodiments, a PBMC immunoprofile type for the subject may be identified by associating the PBMC signature of the subject with a particular one of the plurality of PBMC signature clusters (e.g., the type identified may be the type associated with the PBMC signature cluster to which the PBMC signature of the subject is closest according to a distance measure or any suitable measure of distance or similarity); and identifying the PBMC immunoprofile type for the subject as the PBMC immunoprofile type corresponding to the particular one of the plurality of PBMC signature clusters to which the PBMC signature of the subject is associated. Examples of PBMC immunoprofile types are described herein.

As described above, a subject's PBMC immunoprofile type is identified at act 652. In some embodiments, the PBMC immunoprofile type of a subject is identified to be one of the following PBMC immunoprofile types: Naive type (G1), Primed type (G2), Progressive type (G3), Chronic type (G4), or Suppressive type (G5). In some embodiments, process 640 ends once act 652 is complete.

TABLE 5
Exemplary cell types used in PBMC signatures.

HLA-DR-Tcells
CD4 T cells
Th1 CD4 T cells
Th2 CD4 T cells
Th17 CD4 T cells
CD4 Naïve T cells
CD4 Naïve Tregs
CD4 Memory T helpers
CD4 Effector Memory
CD4 Central Memory
CD4 TEMRA
CD8 T cells
CD8 Naïve T cells
CD8 Memory T cells
CD8 Transitional Memory PD-1+
CD8 Transitional Memory
CD8 Central Memory
CD8 Effector Memory
Follicular T cells
CD8 TEMRA
CD8 TEMRA PD-1+
Non-switched Memory IgM B
cells
Class-switched Memory
Naïve B cells
Classical Monocytes
Non-classical Monocytes
Mature NK cells
Immature NK cells
Dendritic cells
Plasmacytoid Dendritic cells
cDC2
NKT cells
Basophils
Eosinophils
Neutrophils
Granulocytes

TABLE 6
Exemplary cell types used in PBMC signatures.

CD4 T cells
CD4 Naïve T cells
CD4 Naïve Tregs
CD4 Memory T helpers
CD4 Effector Memory
CD4 Central Memory
CD4 TEMRA
CD8 T cells
CD8 Naïve T cells
CD8 Memory T cells
CD8 Transitional Memory
CD8 Central Memory
CD8 Effector Memory
CD8 TEMRA
Non-switched Memory IgM B
cells
Class-switched Memory
Naïve B cells
Classical Monocytes
Non-classical Monocytes
Mature NK cells
Immature NK cells
Dendritic cells
Plasmacytoid Dendritic cells
NKT cells
Granulocytes
Neutrophils
Basophils
Eosinophils
CD4 Tregs
CD4 Transitional Memory
HLA DR low Monocytes
TIGIT+ PD1+ CD8 T cells
CD39 CD4 Tregs
gdT Vdelta2+

TABLE 7
Exemplary cell types used in PBMC signatures.

CD4 T cells
CD4 Naïve T cells
CD4 Naïve Tregs
CD4 Memory T helpers
CD4 Effector Memory
CD4 Central Memory
CD4 TEMRA
CD8 T cells
CD8 Naïve T cells
CD8 Memory T cells
CD8 Transitional Memory
CD8 Central Memory
CD8 Effector Memory
CD8 TEMRA
Non-switched Memory IgM B
cells
Class-switched Memory
Naïve B cells
Classical Monocytes
Non-classical Monocytes
Mature NK cells
Immature NK cells
Dendritic cells
Plasmacytoid Dendritic cells
NKT cells
Granulocytes
Neutrophils
Basophils
Eosinophils
CD4 Tregs
CD4 Transitional Memory
HLA DR low Monocytes
TIGIT+ PD1+ CD8 T cells
CD39 CD4 Tregs
gdT Vdelta2+
HLA-DR-Tcells
Th1 CD4 T cells
Th2 CD4 T cells
Th17 CD4 T cells
CD8 Transitional Memory PD-1+
Follicular T cells
CD8 TEMRA PD-1+
cDC2

### Cell Composition Percentages

Aspects of the disclosure relate to determining a G2 score for a blood sample by processing cell population data or RNA expression data to obtain cell composition percentages. As used herein, a “cell composition percentage” refers to the percentage of a particular cell type in a plurality of cells. For example, if 100 cells of a total cell population of 500 cells are identified as being CD4 T cells, the cell composition percentage of CD4 T cells in the population is 20%.

Cell composition percentages can be determined using different techniques. The technique may depend on the type of data obtained for the blood sample. For example, different techniques may be used to obtain cell composition percentages given the following types of data: cytometry data, RNA expression data, hematology data, DNA methylation data, and MxIF image data. Examples of techniques for determining cell composition percentages (“deconvolution”) are described herein. However, it should be appreciated that the techniques developed by the inventors are not limited to any particular deconvolution technique, and any suitable deconvolution technique may be used to determine the cell composition percentages of cell types in the blood sample.

### Cytometry-Based Cell Composition Percentages

In some embodiments, cell composition percentages are determined using cytometry data obtained for a blood sample. For example, this may include applying one or more machine learning models to the cytometry data to obtain cell composition percentages for the cell types. Examples of machine learning models that may be used to process cell population data to obtain cell composition percentages are described, for example in International Application No PCT/US2023/012003, filed Jan. 31, 2023, the entire contents of which are incorporated by reference herein. Additionally or alternatively, the cell composition percentages may be determined based on cell counts specified in the cytometry data for different cell types. For example, the cytometry data may processed (e.g., by gating) to determine the cell counts. Determining the cell composition percentage for a particular cell type may include determining a ratio of the number of cells of the particular cell type to a total number of cells specified for the sample. In some embodiments, the cytometry data may be processed to obtain cell composition percentages for at least some (e.g., all) of the cell types listed in Table 2. Additionally or alternatively, the cytometry data may be processed to obtain a cell composition percentage of peripheral mononuclear cells (PBMCs) in the blood sample.

FIG. 7 is a flowchart of process 700, which may be used to implement act 428 shown in FIG. 4B (and is therefore an example implementation of act 428) for determining cell composition percentages using cytometry data. Process 700 may be performed in part or in full by a laptop computer, a desktop computer, one or more servers, in a cloud computing environment, computing device as described herein with respect to FIG. 14 or using any other suitable computing device(s), as aspects of the technology described herein are not limited in this respect.

Process 700 begins at act 702 for obtaining cytometry data for a biological sample from a subject, the biological sample including a plurality of cells. In some embodiments, act 702 may be performed in any suitable way such as, for example, as described herein including at least with respect to act 308 of process 300 shown in FIG. 3A and/or act 358 of process 350 shown in FIG. 3B. For example, cytometry (e.g., flow cytometry, mass cytometry, spectral cytometry, etc.) may be performed on the biological sample (e.g., using any suitable flow cytometry device or platform) to obtain the cytometry data.

Next, at act 704, a respective type is identified for each of at least some of the plurality of cells based on the cytometry data obtained at act 702. In some embodiments, act 704 may be performed according to the techniques described herein including at least with respect to FIG. 4B for identifying types for cells in a biological sample.

Next, at act 706, a cell count is determined for each of multiple cell types identified at act 704. In some embodiments, this includes determining a number of cells, or cell count, of each type of cell for which cytometry measurements are obtained at act 702. The cell counts, in some embodiments, may be used to determine a number of cells of each type of cell included in at least a hierarchy of cell types. A hierarchy of cell types may indicate relationships between different cell types. For example, the hierarchy of cell types may include parent cell types and cell types that are children, or subtypes, of the parent cell type. In some embodiments, data indicating a hierarchy of cell types is received as input at act 706. Such data may be provided in any suitable format, as aspects of the technology described herein are not limited in this respect.

In some embodiments, data indicating the types identified (at act 704) for each of multiple cells in the biological sample may also be received at act 706. For example, the input may include a tab-separated values file having a number of lines corresponding to the number of objects. Each of at least some of the lines may include an indication of the type determined for the cell. In some embodiments, at least some of the cell types indicated for the cells are included in the hierarchy of cell types. In some embodiments, one or more cell types are not included in the hierarchy of cell types. For example, the identified cell types may include types for “doubles,” which are a combination of two different cell types (e.g., “Monocytes & Neutrophils”). As another example, the identified cell types may include one or more custom cell types which one or more of machine learning models were trained to predict (e.g., “Dead Neutrophils”).

In some embodiments, a “raw” cell count is determined for each unique cell type listed in the data indicating the types identified for the subsample. For example, this includes determining counts for types that are included in the hierarchy of cell types and types that are not included in the hierarchy of cell types.

In some embodiments, the determined cell counts are then updated to conform with cell types included in the hierarchy of cell types. For example, this may include attributing a cell count determined for an identified cell type that is not included in the hierarchy to a cell type that is included in the hierarchy. For example, a cell count determined for the identified cell type of “Dead Neutrophils,” which is not included in the hierarchy, may be attributed to the cell type “Neutrophils,” which is included in the hierarchy. For example, the cell count may be added to the cell count for neutrophils. Accordingly, in some embodiments, since the cell count is accounted for by the “Neutrophil” cell type, the cell count for “Dead Neutrophils” may be discarded. In some embodiments, in updating the determined cell counts to conform with cell types included in the hierarchy of cell types, “doubles” may also be split into two different cell types, and cell counts may be updated for the respective cell types accordingly. For example, a count of “Monocytes & Neutrophils”) may be split into a count of Monocytes and a count of Neutrophils. Accordingly, in some embodiments, any existing cell counts for Monocytes and Neutrophils may be updated to include said counts. Since the cell counts are accounted for by the “Monocyte” and “Neutrophil” cell type, the cell count for “Monocyte & Neutrophil” may be discarded.

In some embodiments, cell counts for parent cell types in the hierarchy of cell types are determined as a sum of the cell counts of their descendants (e.g., subtypes). For example, a cell that is identified to be a “Classical Monocyte” is also a “Monocyte,” since “Classical Monocyte” is a subtype of “Monocyte.” Accordingly, in some embodiments, the cell count of a parent cell type in the hierarchy of cell types may be updated based on the cell counts of its descendants. For example, the cell counts of the descendants may be added to an existing cell count for the parent or added from zero, if there is no existing cell count for the parent cell type. In some embodiments, the techniques for updating cell counts of parent cell types may be carried out sequentially from the bottom of the hierarchy of cell types to the top of the hierarchy of cell types.

Next, at act 708, a cell composition percentage is determined for each of at least some of the identified cell types. In some embodiments, determining a cell composition percentage for a particular cell type includes determining a ratio between the number of cells of a particular type and a total number of cells determined for the biological sample. In some embodiments, determining a cell composition percentage for a particular cell type includes determining a ratio between the number of cells of a particular type and a total number of immune cells determined for the biological sample. In some embodiments, determining a cell composition percentage for a particular cell type includes determining, in the biological sample, a percentage of the particular cell type relative to a cell type class associated with the particular cell type. For example, determining the percentage of naïve T cells relative to the total number of T cells identified in the biological sample. For example, the total number of cells may be determined as the number of leukocytes determined for the biological sample.

In some embodiments, the cell composition percentages determined for particular cell types are used to determine cell concentrations of those cell types in the biological sample. For example, the normalized cell composition percentages may be multiplied by a respective coefficient that converts the cell composition percentage to a cell concentration.

### Expression-Based Cell Composition Percentages

In some embodiments, cell composition percentages are determined using RNA expression data obtained for a blood sample. For example, the cell composition percentages may be determined using one or more cell deconvolution techniques to generate cell composition percentages for one or more cell types (e.g., some (or all) of the cell types listed in Table 2, Table 3, Table 4, Table 5, Table 6 and/or Table 7). The use of cell deconvolution techniques, for example the BostonGene Kassandra technique, to generate cell composition percentages has been described, for example by International Application No. PCT/US2021/022155, published as International Publication No. WO2021/183917 on Sep. 16, 2021; and International Application No. PCT/US2022/027088, published as International Publication No. WO2022/232615 on Nov. 3, 2022, the entire contents of each of which are incorporated by reference herein. Other cell deconvolution techniques may also be used in methods described by the disclosure, for example Cibersort (e.g., as described by Newman et al. Nature Methods volume 12, pages 453-457 (2015)) or CibersortX (e.g., as described by Newman et al. Nature Biotechnology volume 37, pages 773-782 (2019)). In some embodiments, more than one cell deconvolution approach is used and then a consensus from the more than one cell devolution approach is used to determine the cell deconvolution.

In some embodiments, the cell composition percentages are adjusted based on a hierarchy of cell types. For example, one or more cell compositions for different cell types may be reconciled with one another.

### DNA Methylation-Based Cell Composition Percentages

In some embodiments, cell composition percentages are determined using DNA methylation data obtained for the blood sample. For example, the cell composition percentages may be determined using a reference-based or a reference-free deconvolution algorithm. An example of a reference-based algorithm is described by Houseman, et al. (Reference-free deconvolution of DNA methylation data and mediation by cell composition effects. BMC Bioinformatics, 17, 259, (2016)), which is incorporated by reference herein in its entirety. Example of reference-free deconvolution algorithms are described by Zou et al. (Epigenome-wide association studies without the need for cell-type composition. Nat. Meth., 11, 309-311, (2014)) and Houseman, et al. (Reference-free cell mixture adjustments in analysis of DNA methylation data. Bioinformatics, 1431-1439, (2014).), each of which is incorporated by reference herein in its entirety.

In some embodiments, the cell composition percentages are adjusted based on a hierarchy of cell types. For example, one or more cell compositions for different cell types may be reconciled with one another.

### Hematology-based Cell Composition Percentages

In some embodiments, cell composition percentages are determined using hematology data obtained for a blood sample. For example, the cell composition percentages may be determined based on cell counts specified in the hematology data for different cell types. For example, determining a cell composition percentage for a particular cell type may include determining a ratio of the number of cells of the particular cell type to a total number of cells specified for the sample. In some embodiments, the hematology data may be processed to obtain cell composition percentages for at least some (e.g., all) of the cell types listed in Table 4.

In some embodiments, the cell composition percentages are adjusted based on a hierarchy of cell types. For example, one or more cell compositions for different cell types may be reconciled with one another.

### MxIF-Based Cell Composition Percentages

In some embodiments, cell composition percentages are determined using MxIF image data. Example techniques for determining cell composition percentages using MxIF images are described at least by International Application No. PCT/US2021/021265, published as International Publication No. WO2021/178938 on Sep. 10, 2021, and which is incorporated by reference herein in its entirety.

In some embodiments, the cell composition percentages are adjusted based on a hierarchy of cell types. For example, one or more cell compositions for different cell types may be reconciled with one another.

### MF Profile Types

In some embodiments, a tumor microenvironment (TME) may be characterized or classified as one of four molecular functional (MF) profile types, herein identified as the first MF profile type, second MF profile type, third MF profile type, and fourth MF profile type. As used herein, the term “MF profile type” refers to a TME having certain features including certain gene expression levels, gene group expression levels, molecular and cellular compositions, and/or biological processes.

TMEs of the first MF profile type may also be described as “inflamed/vascularized” and/or “inflamed/fibroblast-enriched” and/or “immune-enriched/fibrotic”; TMEs of the second MF profile type may also be described as “inflamed/non-vascularized” and/or “inflamed/non-fibroblast-enriched”and/or “immune-enriched/non-fibrotic”; TMEs of the third MF profile type may also be described as “non-inflamed/vascularized” and/or “non-inflamed/fibroblast-enriched” and/or “fibrotic”; and TMEs of the fourth MF profile type may also be described as “non-inflamed/non-vascularized” and/or “non-inflamed/non-fibroblast-enriched” and/or “immune desert.”

The MF profile types may additionally or alternatively be characterized based on training samples. For example, training samples may be assigned to one of four MF profile clusters using a classifier (e.g., a k-nearest classifier). The classifier may be trained on the data by which the MF profile clusters are defined and on their corresponding labels. The classifier may then predict the type of MF profile (MF profile cluster) for the subject sample utilizing its relative processes intensity values. Relative processes intensity values may be calculated as Z-values (arguments of the standard normal distribution over training set of samples) of single sample GSEA algorithm outputs inferred from the RNA sequence data from the subject sample. For example, the Z-values may include the NK cell z-score, T cell z-score, angiogenesis z-score, fibroblast z-score, referred to herein.

As used herein, “inflamed” refers to the gene and/or gene group expression related to inflammation in a TME. For example, “inflamed” may refer to a high level of gene or gene group expression associated with inflammation (e.g., higher than non-inflamed MF profiles). In some embodiments, inflamed TMEs are highly infiltrated by immune cells, and are highly active with regard to antigen presentation and T-cell activation. In some embodiments, inflamed TMEs may have an NK cell and/or a T cell z score of, for example, at least 0.60, at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, or at least 0.99. In some embodiments, inflamed TMEs may have an NK cell and/or a T cell z score of, for example, not less than 0.60, not less than 0.65, not less than 0.70, not less than 0.75, not less than 0.80, not less than 0.85, not less than 0.90, not less than 0.91, not less than 0.92, not less than 0.93, not less than 0.94, not less than 0.95, not less than 0.96, not less than 0.97, not less than 0.98, or not less than 0.99. In some embodiments, non-inflamed tumors are poorly infiltrated by immune cells, and have low activity with regard to antigen presentation and T-cell activation. In some embodiments, non-inflamed TMEs may have an NK cell and/or a T cell z score of, for example, less than −0.20, less than −0.25, less than −0.30, less than −0.35, less than −0.40, less than −0.45, less than −0.50, less than −0.55, less than −0.60, less than −0.65, less than −0.70, less than −0.75, less than −0.80, less than −0.85, less than −0.90, less than −0.91, less than −0.92, less than −0.93, less than −0.94, less than −0.95, less than −0.96, less than −0.97, less than −0.98, or less than −0.99. In some embodiments, non-inflamed TMEs may have an NK cell and/or a T cell z score of, for example, not more than −0.20, not more than −0.25, not more than −0.30, not more than −0.35, not more than −0.40, not more than −0.45, not more than −0.50, not more than −0.55, not more than −0.60, not more than −0.65, not more than −0.70, not more than −0.75, not more than −0.80, not more than −0.85, not more than −0.90, not more than −0.91, not more than −0.92, not more than −0.93, not more than −0.94, not more than −0.95, not more than −0.96, not more than −0.97, not more than −0.98, or not more than −0.99.

As used herein, “vascularized” refers to the formation of blood vessels in a TME. In some embodiments, vascularized TMEs comprise high levels of gene and/or gene group expression related to cellular compositions and process related to blood vessel formation. For example, the gene and/or gene group expression levels related to blood vessel formation may be higher in vascularized TMEs compared to non-vascularized TMEs. In some embodiments, vascularized TMEs may have an angiogenesis z score of, for example, at least 0.60, at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, or at least 0.99. In some embodiments, vascularized TMEs may have an NK cell and/or a T cell z score of, for example, not less than 0.60, not less than 0.65, not less than 0.70, not less than 0.75, not less than 0.80, not less than 0.85, not less than 0.90, not less than 0.91, not less than 0.92, not less than 0.93, not less than 0.94, not less than 0.95, not less than 0.96, not less than 0.97, not less than 0.98, or not less than 0.99. In some embodiments, in non-vascularized TMEs, gene and/or gene group expression levels related to compositions and processes related to blood vessel formation are relatively low (e.g., compared to in vascularized TMEs). In some embodiments, non-vascularized TMEs may have an angiogenesis z score of, for example, less than −0.20, less than −0.25, less than −0.30, less than −0.35, less than −0.40, less than −0.45, less than −0.50, less than −0.55, less than −0.60, less than −0.65, less than −0.70, less than −0.75, less than −0.80, less than −0.85, less than −0.90, less than −0.91, less than −0.92, less than −0.93, less than −0.94, less than −0.95, less than −0.96, less than −0.97, less than −0.98, or less than −0.99. In some embodiments, non-vascularized TMEs may have an angiogenesis z score of, for example, not more than −0.20, not more than −0.25, not more than −0.30, not more than −0.35, not more than −0.40, not more than −0.45, not more than −0.50, not more than −0.55, not more than −0.60, not more than −0.65, not more than −0.70, not more than −0.75, not more than −0.80, not more than −0.85, not more than −0.90, not more than −0.91, not more than −0.92, not more than −0.93, not more than −0.94, not more than −0.95, not more than −0.96, not more than −0.97, not more than −0.98, or not more than −0.99.

As used herein, “fibroblast enriched” refers to the level or amount of fibroblasts in a TME. In some embodiments, fibroblast enriched tumors comprise high levels of fibroblast cells compared to non-fibroblast enriched tumors. In some embodiments, fibroblast enriched TMEs may have a fibroblast (cancer associated fibroblast) z score of, for example, at least 0.60, at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, or at least 0.99. In some embodiments, fibroblast enriched cancers (e.g., tumors) may have an NK cell and/or a T cell z score of, for example, not less than 0.60, not less than 0.65, not less than 0.70, not less than 0.75, not less than 0.80, not less than 0.85, not less than 0.90, not less than 0.91, not less than 0.92, not less than 0.93, not less than 0.94, not less than 0.95, not less than 0.96, not less than 0.97, not less than 0.98, or not less than 0.99. In some embodiments, non-fibroblast-enriched TMEs comprise few or no fibroblast cells. In some embodiments, non-fibroblast-enriched TMEs may have a fibroblast (cancer associated fibroblast) z score of, for example, less than −0.20, less than −0.25, less than −0.30, less than −0.35, less than −0.40, less than −0.45, less than −0.50, less than −0.55, less than −0.60, less than −0.65, less than −0.70, less than −0.75, less than −0.80, less than −0.85, less than −0.90, less than −0.91, less than −0.92, less than −0.93, less than −0.94, less than −0.95, less than −0.96, less than −0.97, less than −0.98, or less than −0.99. In some embodiments, non-fibroblast-enriched cancers (e.g., tumors) may have a fibroblast (cancer associated fibroblast) z score of, for example, not more than −0.20, not more than −0.25, not more than −0.30, not more than −0.35, not more than −0.40, not more than −0.45, not more than −0.50, not more than −0.55, not more than −0.60, not more than −0.65, not more than −0.70, not more than −0.75, not more than −0.80, not more than −0.85, not more than −0.90, not more than −0.91, not more than −0.92, not more than −0.93, not more than −0.94, not more than −0.95, not more than −0.96, not more than −0.97, not more than −0.98, or not more than −0.99.

### Selecting MF Profile Types

Aspects of the disclosure relate to selecting an MF profile type for a subject by processing RNA expression data obtained for a tumor sample obtained for the subject. Example techniques for identifying MF profile types for a biological sample have been described by Bagaev, A., et al. (“Conserved pan-cancer microenvironment subtypes predict response to immunotherapy.” Cancer cell 39.6 (2021): 845-865) and in International Application No. PCT/US2018/037017, published as International Publication No. WO2018/231771 on Dec. 20, 2018, the entire contents of each of which are incorporated by reference herein in its entirety.

FIG. 8A is a flowchart of an illustrative computer-implemented process 800 for identifying a MF profile cluster with which to associate an MF profile for a subject (e.g., a cancer patient), in accordance with some embodiments of the technology described herein. In some embodiments, process 800 may be used to implement act 304 shown in FIG. 3A and/or act 354 shown in FIG. 3B (and is therefore an example implementation of act 304 and/or act 354) for selecting an MF profile type for a tumor sample. Process 800 may be performed in part or in full by a laptop computer, a desktop computer, one or more servers, in a cloud computing environment, computing device as described herein with respect to FIG. 14 or using any other suitable computing device(s), as aspects of the technology described herein are not limited in this respect.

Process 800 begins at act 802, where RNA expression data is obtained for a tumor sample from a subject. The RNA expression data may be obtained using any of the techniques described herein including at least with respect to act 302 of process 300 shown in FIG. 3A and act 352 shown in FIG. 3B.

Next, process 800 proceeds to act 804, where the MF profile for the subject is determined by determining a set of expression levels for a respective set of gene groups. The MF profile may be determined for a subject having any type of cancer, including any of the types described herein. The MF profile may be determined using any number of gene groups that relate to compositions and processes present within and/or surrounding the subject's tumor. In some embodiments, the MF profile includes a vector of gene group expression levels for respective gene groups. Further aspects relating to determining MF profiles are provided in section titled “MF Profiles”.

Next, process 800 proceeds to act 806, where a MF profile cluster with which to associate the MF profile of the subject is identified. The MF profile of the subject may be associated with any of the types of MF profile cluster types described herein. A subject's MF profile may be associated with one or multiple of the MF profile clusters in any suitable way. For example, an MF profile may be associated with one of the MF profile clusters using a similarity metric (e.g., by associating the MF profile with the MF profile cluster whose centroid is closest to the MF profile according to the similarity metric). As another example, a statistical classifier (e.g., k-means classifier or any other suitable type of statistical classifier) may be trained to classify the MF profile as belonging to one or multiple of the MF clusters. Further aspects relating to determining MF profiles are provided in section “MF Profiles”.

FIG. 8B is a flowchart of an illustrative computer-implemented process 820 for generating MF profile clusters using expression data obtained from subjects having a particular type of cancer, in accordance with some embodiments of the technology described herein. MF profile clusters may be generated for any cancer using expression data obtained from patients having that type of cancer. For example, MF profile clusters associated with melanoma may be generated using expression data from melanoma patients. In another example MF profile clusters associated with lung cancer may be generated using expression data from lung cancer patients.

Process 820 begins at act 822, where RNA expression data for a plurality of subjects having a particular cancer are obtained. The plurality of subjects for which expression data is obtained may comprise any number of patients having a particular cancer. For example, expression data may be obtained for a plurality of melanoma patients, for example, 100 melanoma patients, 1000 melanoma patients, or any number of melanoma patients as the technology is not so limited. RNA expression data may be acquired using any method known in the art, e.g., whole transcriptome sequencing, total RNA sequencing, and mRNA sequencing. Further aspects relating to obtaining expression data are provided in section “Sequencing Data”.

Next, process 820 proceeds to act 824, where the MF profile for each subject in the plurality of subject is determined by determining a set of expression levels for a respective set of gene groups. For example, the MF profile may be a vector having values corresponding to the expression levels for the gene groups. MF profiles may be determined using any number of gene groups that relate to compositions and processes present within and/or surrounding the subject's tumor. Gene group expression levels, in some embodiments, may be calculated as a gene set enrichment (GSEA) score for the gene group. Further aspects relating to determining MF profiles are provided in section titled “MF Profiles”.

Next, process 820 proceeds to act 826, where the plurality of MF profiles are clustered to obtain MF profile clusters. MF profiles may be clustered using any of the techniques described herein including, for example, community detection clustering, dense clustering, k-means clustering, or hierarchical clustering. MF profiles may be clustered for any type of cancer using MF profiles generated for patients having that type of cancer. MF profile clusters, in some embodiments, comprise a 1st MF profile cluster, a 2nd MF profile cluster, a 3rd MF profile, and a 4th MF profile. The relative sizes of 1st-4th MF clusters may vary among cancer types. Further aspects relating to MF profile clusters are provided in section titled “MF profiles”.

Next, process 820 proceeds to act 828, where the plurality of MF profiles in association with information identifying the particular cancer type are stored. MF profiles may be stored in a database in any suitable format and/or using any suitable data structure(s), as aspects of the technology described herein are not limited in this respect. The database may store data in any suitable way, for example, one or more databases and/or one or more files. The database may be a single database or multiple databases.

In this way, MF profile clusters can be stored and used as existing MF profile clusters with which a patient's MF profile can be associated.

### MF Profiles

As described herein, in some embodiments, an MF profile type may be identified for a subject by (a) determining an MF profile for the subject, and (b) determining an MF profile cluster for the subject based on the MF profile. In some embodiments, MF profile clusters are obtained by (a) determining MF profiles for a plurality of subjects, and (b) clustering the MF profiles to obtain the MF profile clusters.

In some embodiments, determining an MF profile for a subject includes determining expression levels for genes in one or more gene groups. In some embodiments, the one or more gene groups are selected from Table 8. In some embodiments, the one or more gene groups selected from Table 8 include at least some (e.g., all) of the gene groups listed in Table 8. For example, the one or more gene groups may include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the gene groups listed in Table 8. Additionally, or alternatively, the one or more gene groups may include at most 20, at most 19, at most 18, at most 17, at most 16, at most 15, at most 14, at most 14, at most 13, at most 12, at most 11, at most 10, at most 9, at most 8, at most 7, at most 6, at most 5, at most 4, at most 3, at most 2, or at most 1 of the gene groups listed in Table 8.

In some embodiments, determining expression levels for genes in a particular gene group listed in Table 8 includes determining an expression level for at least some (e.g., all) of the genes listed for that particular gene group.

In some embodiments, the one or more gene groups are selected from the gene groups listed in International Application No. PCT/US2018/037017, published as International Publication No. WO2018/231771 on Dec. 20, 2018, which is incorporated by reference herein in its entirety. In some embodiments, the one or more gene groups selected from the gene groups listed in International Application No. PCT/US2018/037017 include at least some (e.g., all) of the gene groups listed in International Application No. PCT/US2018/037017. For example, the one or more gene groups may include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 21, at least 22, at least 23, at least 24, at least 25, at least 26, at least 27, or at least 28 of the gene groups listed in International Application No. PCT/US2018/037017. Additionally, or alternatively, the one or more gene groups may include at most 27, at most 26, at most 25, at most 24, at most 23, at most 22, at most 21, at most 20, at most 19, at most 18, at most 17, at most 16, at most 15, at most 14, at most 14, at most 13, at most 12, at most 11, at most 10, at most 9, at most 8, at most 7, at most 6, at most 5, at most 4, at most 3, at most 2, or at most 1 of the gene groups listed in International Application No. PCT/US2018/037017.

In some embodiments, determining expression levels for genes in a particular gene group listed in International Application No. PCT/US2018/037017 includes determining an expression level for at least some (e.g., at least three, at least four, at least five, at least six, at least seven, at least eight, at least nine, at least ten, all) of the genes listed for that particular gene group.

In some embodiments, the one or more gene groups are selected from the gene groups described by Bagacv, A., et al. (“Conserved pan-cancer microenvironment subtypes predict response to immunotherapy.” Cancer cell 39.6 (2021): 845-865), which is incorporated by reference herein in its entirety. In some embodiments, the one or more gene groups selected from the gene groups described by Bagaev, et al. include at least some (e.g., all) of the gene groups described by Bagaev, et al. For example, the one or more gene groups may include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 21, at least 22, at least 23, at least 24, at least 25, at least 26, at least 27, or at least 28 of the gene groups described by Bagaev, et al. Additionally, or alternatively, the one or more gene groups may include at most 27, at most 26, at most 25, at most 24, at most 23, at most 22, at most 21, at most 20, at most 19, at most 18, at most 17, at most 16, at most 15, at most 14, at most 14, at most 13, at most 12, at most 11, at most 10, at most 9, at most 8, at most 7, at most 6, at most 5, at most 4, at most 3, at most 2, or at most 1 of the gene groups described by Bagaev, et al.

In some embodiments, determining expression levels for genes in a particular gene group described by Bagaev, et al. (“Conserved pan-cancer microenvironment subtypes predict response to immunotherapy.” Cancer cell 39.6 (2021): 845-865) includes determining an expression level for at least some (e.g., at least three, at least four, at least five, at least six, at least seven, at least eight, at least nine, at least ten, all) of the genes listed for that particular gene group.

In some embodiments, the expression levels for genes in a particular gene group are used to determine a gene group expression level for the gene group. For example, a gene group expression level may be determined for at least some (e.g., all) of the gene groups listed in Table 8. Additionally or alternatively, a gene group's expression level may be determined for at least some (e.g., all) of the gene groups listed in International Application No. PCT/US2018/037017. Additionally or alternatively, a gene group expression level may be determined for at least some (e.g., all) of the gene groups described by Bagaev, et al. (“Conserved pan-cancer microenvironment subtypes predict response to immunotherapy.” Cancer cell 39.6 (2021): 845-865).

In some embodiments, a gene group expression level is a summarized expression score based on expression levels of at least some genes in the gene group. For example, a gene group expression level may be determined using a gene set enrichment analysis (GSEA) technique.

In some embodiments, an MF profile is generated using a plurality of gene group expression levels. For example, the MF profile may comprise a vector of the plurality of gene group expression levels.

TABLE 8
Example gene groups and genes in each example gene group.

| Gene Group | Genes |
|---|---|
| Angiogenesis | CDH5, VWF, ANGPT2, CXCR2, VEGFB, PDGFC, |
| | CXCL8, VEGFC, ANGPT1, FLT1, VEGFA, TEK, |
| | PGF, CXCL5, KDR |
| Endothelium | CDH5, VWF, ENG, VCAM1, FLT1, CLEC14A, |
| | KDR, NOS3, MMRN2, MMRN1 |
| CAF | FGF2, MFAP5, COL1A1, COL5A1, FAP, PDGFRB, |
| | FBLN1, CD248, COL6A2, ACTA2, MMP3, |
| | COL6A3, COL1A2, PDGFRA, LRP1, CXCL12, |
| | COL6A1, LUM, MMP2 |
| Matrix | COL3A1, LGALS9, TNC, LAMA3, COL11A1, |
| | COL1A1, ELN, LGALS7, VTN, COL5A1, LAMB3, |
| | LAMC2, COL4A1, COL1A2, FN1 |
| Matrix | MMP7, MMP3, MMP9, CA9, ADAMTS4, MMP1, |
| remodeling | ADAMTS5, MMP11, PLOD2, LOX, MMP12, MMP2 |
| Macrophages | MSR1, CD68, MRC1, SIGLEC1, CSF1R, IL4I1, |
| | CD163, IL10 |
| Macrophage | CCL2, CCL7, CCL8, CSF1, CCR2, CSF1R, XCL1, |
| DC traffic | XCR1 |
| MDSC | IDO1, IL4I1, IL10, ARG1, PTGS2, CYBB, IL6 |
| Treg | FOXP3, TNFRSF18, IKZF4, IL10, CCR8, IKZF2, |
| | CTLA4 |
| M1 signatures | IL1B, IRF5, IL23A, IL12B, NOS2, TNF, IL12A, |
| | SOCS3, CMKLR1 |
| MHCII | HLA-DRA, HLA-DRB1, HLA-DPA1, HLA-DQB1, |
| | HLA-DPB1, HLA-DMB, CIITA, HLA-DQA1, HLA- |
| | DMA |
| Antitumor | TNF, IL21, IFNA2, TNFSF10, CCL3, IFNB1 |
| cytokines | |
| B cells | FCRL5, STAP1, CR2, CD19, TNFRSF13C, CD79A, |
| | CD22, TNFRSF13B, CD79B, TNFRSF17, BLK, |
| | PAX5, MS4A1 |
| NK cells | CD160, FGFBP2, KLRK1, GNLY, IFNG, KIR2DL4, |
| | NCR3, CD226, GZMB, GZMH, CD244, NCR1, |
| | EOMES, KLRF1, NKG7, SH2D1B, KLRC2 |
| Checkpoint | LAG3, CD274, PDCD1, BTLA, VSIR, CTLA4, |
| inhibition | PDCD1LG2, TIGIT, HAVCR2 |
| Effector cells | CD8A, FASLG, ZAP70, GNLY, TBX21, GZMA, |
| | IFNG, EOMES, PRF1, GZMK, GZMB, CD8B |
| T cells | CD3G, CD5, CD28, TBX21, CD3E, TRBC2, TRAT1, |
| | CD3D, ITK, TRBC1, TRAC |
| T cell traffic | CXCL9, CCL5, CXCL10, CXCL11, CX3CL1, CCL3, |
| | CXCR3, CX3CR1, CCL4 |
| MHCI | HLA-A, HLA-B, TAPBP, B2M, HLA-C, NLRC5, |
| | TAP1, TAP2 |
| EMT signature | TWIST2, ZEB1, SNAI2, SNAI1, TWIST1, ZEB2, |
| | CDH2 |
| Proliferation | MCM6, CCNE1, ESCO2, MYBL2, AURKB, E2F1, |
| rate | CCND1, CDK2, BUB1, CETN3, AURKA, CCNB1, |
| | MCM2, MKI67, PLK1 |

### EXAMPLES

### Example 1—Predicting Response to Anti-PD-1 in Human Papillomavirus Negative Head and Neck Squamous Cell Carcinomas (HPV-HNSCC)

This example shows that immunoprofiling of PBMCs and RNA-seq of tumor tissue can be used to accurately predict response to anti-PD-1 in human papillomavirus negative head and neck squamous cell carcinomas (HPV-HNSCC).

### Methods

Immunoprofiling with multiparameter flow cytometry was applied to peripheral blood samples from a mixed cohort of healthy donors and cancer patients (n=850). Robust cell populations that were differentially represented in these two groups were selected to train a machine learning (ML)-based classifier and identify groups or immunotypes with putative functional significance. Unsupervised clustering of normalized cell population frequencies from flow cytometry data was used to classify patients into five different immunotypes, which were analytically validated by cellular deconvolution of RNA-seq data with Kassandra. Kassandra is described by Zaitsev, Aleksandr, et al. (“Precise reconstruction of the TME using bulk RNA-seq and a machine learning algorithm trained on artificial transcriptomes.” Cancer Cell 40.8 (2022): 879-894); International Application No. PCT/US2021/022155, published as International Publication No. WO2021/183917 on Sep. 16, 2021; and International Application No. PCT/US2022/027088, published as International Publication No. WO2022/232615 on Nov. 3, 2022, the entire contents of each of which are incorporated by reference herein. PBMC immune cell populations of previously untreated stage II-IV HNSCC patients (n=36) were analyzed at baseline and on-treatment with the anti-PD-1 inhibitor nivolumab. RNA-seq was retrospectively performed on tumors at baseline and on-treatment, along with transcriptomic-based tumor microenvironment (TME) subtyping and cellular deconvolution with Kassandra. All disease sites were assigned a pathologic Treatment Response (pTR) and analysis was completed based on primary site response alone and overall response (OR) based on all disease sites.

### Internal Cohort Description

Peripheral blood samples of cancer patients were collected in multiple medical centers across the United States and delivered to BostonGene Laboratory. Blood of healthy donors were purchased from multiple collection centers around the Research Blood Components (Watertown, MA), STEMCELL Technologies (Vancouver, BC, Canada), and Discovery Life Sciences (Huntsville, AL). All patients provided written consent under IRB-approved protocols. Initially, 960 blood samples were collected for flow cytometry analysis, among them 470 patients with different cancer types (145 with sarcoma cancer subtypes and 325 with cancers of epithelial origin) and 449 healthy donor samples. 145 patients had sarcoma cancer subtype, 325 cancer of epithelial origin. After exclusion of samples based on insufficient quality, a total of 850 flow cytometry samples were analyzed in this study.

The median age in the cohort was 47 years for healthy donors and 61.5 for cancer patients. Only patients with sarcomas and carcinomas were included, with the most frequent epithelial origin diagnoses: Pancreatic cancer (n=37), Breast neoplasm (n=65), Non-small cell lung carcinoma (n=32), Colorectal neoplasm (n=41), Melanoma (n=19) and Prostate (n=18). Therapeutic information was available for 417 (417/442, 94.3%) patients. Previous treatments were administered within a year of blood draw to 211 (211/417, 50.6%) patients including chemotherapy, radiotherapy, ICI or systemic therapy classified otherwise. 234 (234/417, 56.1%) patients were on ongoing therapy during material collection. Based on provided data, 44 (44/417, 10.55%) patients had no evidence of therapy administration after cancer diagnosis. Additionally, 797 RNA samples were analyzed from both healthy and cancer blood donors. This diverse cohort was used for multi-scale analysis of the relationship between cancer and peripheral blood immunity.

### Head and Neck Squamous Cell Carcinoma Cohort

To further investigate the implications of newly discovered immune clusters to cancer immunotherapy, this flow cytometry analytical framework was applied to a cohort of 36 Head and Neck Squamous Cell Carcinoma (HNSCC) patients. The HNSCC cohort was part of a prospective phase II trial conducted in Thomas Jefferson University Hospital. During this trial, patients received anti-PD1 monoclonal antibody treatment (nivolumab) or nivolumab in combination with a specific IDO inhibitor (BMS986205). Pre- and post-treatment cryopreserved PBMCs were thawed and subjected to a multicolor flow cytometry staining. In total, 70 samples were analyzed with two of the patients having only pre-therapy samples due to poor quality of post-treatment PBMCs.

### Blood Samples Processing and White Blood Cell (WBC) Isolation

Upon receipt, all fresh peripheral blood samples underwent a complete blood count using the DxH 500 Hematology Analyzer (Beckman Coulter, Brea, CA). Samples received within 24 hours of collection underwent red blood cell (RBC) lysis of 3 ml whole blood to isolate white blood cells (WBCs) using 42 ml nuclease-free HyPure water mixed with 5 ml 10×RBC lysis buffer (eBioscience). Samples were lysed at RT for 10 minutes, continuously mixing on a tube rotator. Cells were then centrifuged at 300×g for 5 minutes and washed with Sorter Buffer (2% NBCS in PBS+1 mM EDTA).

### Cryopreserved PBMC Thawing

Cryopreserved peripheral blood mononuclear cell (PBMC) samples were stored in a vapor phase liquid nitrogen tank and thawed at 37° C. with premade thawing media (20% NBCS in 500 mL RPMI 1640 media+10 mL HEPES+10 mL PENSTREP+10 mL MEMNEAA+10 mL NAHEP+5 mL GlutaMAX). Prior to thawing, a 15 mL aliquot of thawing media was pre-warmed to 37° C. in a water bath and supplemented with 75 uL DNAse (20 mg/mL) and 75 uL Glutathione (200 mM). Samples were removed from the liquid nitrogen tank and immediately dipped into a 37 C water bath, without submerging the cap in the water. Thawing was visually monitored, samples were swirled in the water bath for ˜1 min until only a small ice crystal remained. Using a wide bore 1 ml pipette, each sample was transferred to an empty 15 mL tube. Pre-warmed, supplemented thawing media was slowly pipette into the tube, gently layering the media over the sample. After 3-4 mLs of layering, warmed media was slowly pipetted directly into the sample and simultaneously swirled until the sample was homogenous. Once homogenous, the sample was topped off with warm, supplemented thawing media until a final volume of 15 mL. PBMC samples were then centrifuged at 300×g for 8 minutes and washed with thawing media at 300×g for 8 minutes before staining.

### Cell Staining and Flow Cytometry

Isolated WBCs or PBMCs were centrifuged at 300×g for 5 minutes, resuspended and blocked with Blocking Buffer (IMDM+10% NBCS+DNAse I (1:200)+Human TrueStain FcX (1:50)+Monocyte Blocker (1:50)+Unlabeled Normal Mouse IgG (1:200)) for 10 minutes at RT. After blocking, each sample was aliquoted into 10 unique wells in 96-well plate, centrifuged at 300×g for 3 minutes to remove supernatant. Each well was stained with Ghost Dye Violet 510 Viability Dye in PBS (1:400, Tonbo) at RT for 10 minutes. After staining with viability dye, 200 μL of Sorter Buffer was added to each well, centrifuged at 300×g for 3 minutes with the supernatant removed subsequently. Samples were stained with 10 custom flow cytometry panels (Table 9) for 20 minutes at RT. Once stained, 200 μL of Sorter Buffer was added to each well, centrifuged at 300×g for 3 minutes followed by supernatant removal. Cells were then fixed in a 1% paraformaldehyde solution (Cytofix/Cytoperm, BD Biosciences) overnight at 4° C. The fixation solution was then washed with Sorter Buffer and resuspended in Acquisition Buffer (PBS+0.5% (w/v) BSA+0.75% (w/v) Glycine+5 mM EDTA+Tween-20 (1:2000)+Sodium Azide (1:100)).

Stained and fixed cells were acquired on the BD FACSCelesta Flow Cytometer. Prior to each acquisition, performance of BD FACSCelesta was checked using CS&T Research Beads (BD Biosciences). Compensation matrix was generated through the FACSDiva software by calculating spectral overlap from single stained controls. Single stained controls were prepared in-house by staining a set of 13 samples of Ultracomp eBeads Compensation Beads (Thermofisher) with unique antibodies in each channel.

TABLE 9
Flow Cytometry Panel Antibodies

| | | | |
|---|---|---|---|
| | Antibody | Antibody | Catalog |
| Reagent | Conjugate | Clone | # |

CP10-Lineage

| | | | |
|---|---|---|---|
| Mouse Anti-Human | AF488 | M5E2 | 301811 |
| CD14 | | | |
| Mouse Anti-Human | BB700 | L138 | 746057 |
| CD13 | | | |
| Mouse Anti-Human | BV421 | 5E8 | 310714 |
| CCR3 | | | |
| Mouse Anti-Human | BV605 | 6H6 | 306026 |
| CD123 | | | |
| Mouse Anti-Human | BV650 | L243 | 307650 |
| HLA-DR | | | |
| Mouse Anti-Human | BV711 | OKT3 | 317328 |
| CD3 | | | |
| Mouse Anti-Human | BV786 | HI30 | 304048 |
| CD45 | | | |
| Mouse Anti-Human | PE | 6/40c | 392904 |
| CD66b | | | |
| Mouse Anti-Human | PE-CF594/ | 5.1H11 | 362544 |
| CD56 | PEDazzle | | |
| Mouse Anti-Human | PE-Cy7 | 3.9 | 301608 |
| CD11c | | | |
| Mouse Anti-Human | PE-Cy5 | HIB19 | 302210 |
| CD19 | | | |

CP16 - Dendritic Cells

| | | | |
|---|---|---|---|
| Mouse Anti-Human | AF488 | AER-37 (CRA-1) | 334640 |
| FceR1 | | | |
| Mouse Anti-Human | BB700 | L138 | 746057 |
| CD13 | | | |
| Mouse Anti-Human | BV421 | L161 | 331526 |
| CD1c | | | |
| Mouse Anti-Human | BV510 | W6D3 | 563141 |
| CD15 | | | |
| Mouse Anti-Human | BV510 | OKT3 | 317332 |
| CD3 | | | |
| Mouse Anti-Human | BV510 | HIB19 | 302242 |
| CD19 | | | |
| Mouse Anti-Human | BV510 | 5E8 | 310721 |
| CCR3 | | | |
| Mouse Anti-Human | BV510 | M-T701 | 563650 |
| CD7 | | | |
| Mouse Anti-Human | BV605 | 6H6 | 306026 |
| CD123 | | | |
| Mouse Anti-Human | BV650 | L243 | 307650 |
| HLA-DR | | | |
| Mouse Anti-Human | BV711 | 3G8 | 302044 |
| CD16 | | | |
| Mouse Anti-Human | BV786 | HI30 | 304048 |
| CD45 | | | |
| Mouse Anti-Human | PE | 8F9 | 353804 |
| CLEC9A | | | |
| Mouse Anti-Human | PE-Daz | M80 | 344120 |
| CD141 | | | |
| Mouse Anti-Human | PE-Cy7 | 3.9 | 301608 |
| CD11c | | | |
| Mouse Anti-Human | PE-Cy5 | M5E2 | 301864 |
| CD14 | | | |

CP22 - B Cells

| | | | |
|---|---|---|---|
| Mouse Anti-Human | BB515 | HIB19 | 564456 |
| CD19 | | | |
| Mouse Anti-Human | BB700 | IA6-2 | 566538 |
| IgD | | | |
| Mouse Anti-Human | BV421 | MI15 | 356516 |
| CD138 | | | |
| Mouse Anti-Human | BV510 | OKT3 | 317332 |
| CD3 | | | |
| Mouse Anti-Human | BV510 | M-T701 | 563650 |
| CD7 | | | |
| Mouse Anti-Human | BV510 | WM15 | 740162 |
| CD13 | | | |
| Mouse Anti-Human | BV605 | G18-145 | 563246 |
| IgG | | | |
| Mouse Anti-Human | BV650 | TU66 | 563681 |
| CD39 | | | |
| Mouse Anti-Human | BV711 | ML5 | 311136 |
| CD24 | | | |
| Mouse Anti-Human | BV786 | HI10a | 564960 |
| CD10 | | | |
| Goat Anti-Human | PE | N/A (goat) | 2050-09 |
| IgA | | | |
| Mouse Anti-Human | PE-Daz | MHM-88 | 314529 |
| IgM | | | |
| Mouse Anti-Human | PE-Cy7 | M-T271 | 356412 |
| CD27 | | | |
| Mouse Anti-Human | PE-Cy5 | HIT2 | 303508 |
| CD38 | | | |

CP23 - Monocytes

| | | | |
|---|---|---|---|
| Mouse Anti-Human | AF488 | M5E2 | 301811 |
| CD14 | | | |
| Mouse Anti-Human | BB700 | M-L13 | 745827 |
| CD9 | | | |
| Mouse Anti-Human | BV421 | 3G8 | 562874 |
| CD16 | | | |
| Mouse Anti-Human | BV510 | OKT3 | 317332 |
| CD3 | | | |
| Mouse Anti-Human | BV510 | 5E8 | 310722 |
| CCR3 | | | |
| Mouse Anti-Human | BV510 | HIB19 | 302242 |
| CD19 | | | |
| Mouse Anti-Human | BV510 | M-T701 | 563650 |
| CD7 | | | |
| Mouse Anti-Human | BV605 | AER-37 | 334628 |
| FceR1 | | (CRA-1) | |
| Mouse Anti-Human | BV650 | L243 | 307650 |
| HLA-DR | | | |
| Mouse Anti-Human | BV711 | WM53 | 303424 |
| CD33 | | | |
| Mouse Anti-Human | BV786 | HI30 | 304048 |
| CD45 | | | |
| Mouse Anti-Human | PE | CD84.1.21 | 326008 |
| CD84 | | | |
| Mouse Anti-Human | PE-Daz | W6D3 | 323038 |
| CD15 | | | |
| Mouse Anti-Human | PE-Cy7 | 7-239 | 346014 |
| CD169 | | | |
| Mouse Anti-Human | PE-Cy5 | 15-2 | 321108 |
| CD206 | | | |

CP26 - Natural Killer Cells (NK Cells)

| | | | |
|---|---|---|---|
| Mouse Anti-Human | AF488 | HI30 | 564585 |
| CD45 | | | |
| Mouse Anti-Human | BB700 | p44-8 | 624381 |
| NKp44 | | | |
| Mouse Anti-Human | BV421 | 3G8 | 562874 |
| CD16 | | | |
| Mouse Anti-Human | BV510 | HIB19 | 302242 |
| CD19 | | | |
| Mouse Anti-Human | BV510 | 6H6 | 306022 |
| CD123 | | | |
| Mouse Anti-Human | BV510 | WM15 | 740162 |
| CD13 | | | |
| Mouse Anti-Human | BV510 | OKT3 | 317332 |
| CD3 | | | |
| Mouse Anti-Human | BV605 | 131411 | 747921 |
| NKG2A | | | |
| Mouse Anti-Human | BV650 | HP-MA4 | 752506 |
| CD158 | | | |
| Mouse Anti-Human | BV711 | 134591 | 748164 |
| NKG2C | | | |
| Mouse Anti-Human | BV786 | QA17A04 | 393329 |
| CD57 | | | |
| Mouse Anti-Human | PE | HP-3G10 | 339904 |
| CD161 | | | |
| Mouse Anti-Human | PE-Dazzle594 | 5.1H11 | 362544 |
| CD56 | | | |
| Mouse Anti-Human | PE-Cy7 | 1D11 | 320812 |
| NKG2D | | | |
| Mouse Anti-Human | PE-Cy5 | eBioH4A3 | 15-1079-42 |
| CD107a | | | |

CP24 - CD8 T Cell Differentiation

| | | | |
|---|---|---|---|
| Mouse Anti-Human | BB515 | M-T271 | 564642 |
| CD27 | | | |
| Mouse Anti-Human | BB700 | RPA-T8 | 566452 |
| CD8 | | | |
| Mouse Anti-Human | BV421 | G025H7 | 353716 |
| CXCR3 | | | |
| Mouse Anti-Human | BV510 | B1 | 331220 |
| gdTCR | | | |
| Mouse Anti-Human | BV510 | L200 | 563094 |
| CD4 | | | |
| Mouse Anti-Human | BV510 | HIB19 | 302242 |
| CD19 | | | |
| Mouse Anti-Human | BV510 | WM15 | 740162 |
| CD13 | | | |
| Mouse Anti-Human | BV605 | OKT3 | 317322 |
| CD3 | | | |
| Mouse Anti-Human | BV650 | DREG-56 | 304832 |
| CD62L | | | |
| Mouse Anti-Human | BV711 | DX2 | 305644 |
| CD95 | | | |
| Mouse Anti-Human | BV786 | QA17A04 | 393329 |
| CD57 | | | |
| Mouse Anti-Human | PE | 2A9-1 | 341604 |
| CX3CR1 | | | |
| Mouse Anti-Human | PE-Cy7 | J252D4 | 356924 |
| CXCR5 | | | |
| Mouse Anti-Human | PE-Cy5 | HI100 | 304110 |
| CD45RA | | | |

CP7 - CD8 T Cell Cancer Biomarker

| | | | |
|---|---|---|---|
| Mouse Anti-Human | BB515 | DX29 | 564549 |
| ICOS | | | |
| Mouse Anti-Human | BB700 | RPA-T8 | 566452 |
| CD8 | | | |
| Mouse Anti-Human | BV421 | F38-2E2 | 345008 |
| Tim-3 | | | |
| Mouse Anti-Human | BV510 | 11F2 | 745026 |
| gdTCR | | | |
| Mouse Anti-Human | BV510 | L200 | 563094 |
| CD4 | | | |
| Mouse Anti-Human | BV510 | HIB19 | 302242 |
| CD19 | | | |
| Mouse Anti-Human | BV510 | WM15 | 740162 |
| CD13 | | | |
| Mouse Anti-Human | BV605 | OKT3 | 317322 |
| CD3 | | | |
| Mouse Anti-Human | BV650 | DREG-56 | 304832 |
| CD62L | | | |
| Mouse Anti-Human | BV711 | M-T271 | 356430 |
| CD27 | | | |
| Mouse Anti-Human | BV786 | 11C3C65 | 369322 |
| Lag-3 | | | |
| Mouse Anti-Human | PE | A15153G | 372704 |
| TIGIT | | | |
| Mouse Anti-Human | PE-Daz | EH12.2H7 | 329940 |
| PD-1 | | | |
| Mouse Anti-Human | PE-Cy7 | A1 | 328212 |
| CD39 | | | |
| Mouse Anti-Human | PE-Cy5 | HI100 | 304110 |
| CD45RA | | | |

CP25 - CD4 Treg Biomarker

| | | | |
|---|---|---|---|
| Mouse Anti-Human | BB515 | BNI3 | 566918 |
| CTLA-4 | | | |
| Mouse Anti-Human | BB700 | L200 | 566479 |
| CD4 | | | |
| Mouse Anti-Human | BV421 | BC96 | 302630 |
| CD25 | | | |
| Mouse Anti-Human | BV510 | 11F2 | 745026 |
| gdTCR | | | |
| Mouse Anti-Human | BV510 | RPA-T8 | 563256 |
| CD8 | | | |
| Mouse Anti-Human | BV510 | HIB19 | 302242 |
| CD19 | | | |
| Mouse Anti-Human | BV510 | WM15 | 740162 |
| CD13 | | | |
| Mouse Anti-Human | BV605 | OKT3 | 317322 |
| CD3 | | | |
| Armenian Hamster | BV650 | C398.4A | 313550 |
| Anti-Human ICOS | | | |
| Mouse Anti-Human | BV711 | M-T271 | 356430 |
| CD27 | | | |
| Mouse Anti-Human | BV786 | 11C3C65 | 369322 |
| Lag3 | | | |
| Mouse Anti-Human | PE | A019D5 | 351340 |
| IL-7RA | | | |
| Mouse Anti-Human | PE-Daz | EH12.2H7 | 329940 |
| PD-1 | | | |
| Mouse Anti-Human | PE-Cy7 | A1 | 328212 |
| CD39 | | | |
| Mouse Anti-Human | PE-Cy5 | HI100 | 304110 |
| CD45RA | | | |

CP8 - CD4 T Cell Differentiation

| | | | |
|---|---|---|---|
| Mouse Anti-Human | BB515 | RPA-T4 | 564419 |
| CD4 | | | |
| Mouse Anti-Human | BB700 | 11A9 | 746139 |
| CCR6 | | | |
| Mouse Anti-Human | BV421 | G025H7 | 353716 |
| CXCR3 | | | |
| Mouse Anti-Human | BV510 | 11F2 | 745026 |
| gdTCR | | | |
| Mouse Anti-Human | BV510 | RPA-T8 | 563256 |
| CD8 | | | |
| Mouse Anti-Human | BV510 | HIB19 | 302242 |
| CD19 | | | |
| Mouse Anti-Human | BV510 | WM15 | 740162 |
| CD13 | | | |
| Mouse Anti-Human | BV605 | OKT3 | 317322 |
| CD3 | | | |
| Mouse Anti-Human | BV650 | DREG-56 | 304832 |
| CD62L | | | |
| Mouse Anti-Human | BV711 | M-T271 | 356430 |
| CD27 | | | |
| Mouse Anti-Human | BV786 | HI100 | 304140 |
| CD45RA | | | |
| Mouse Anti-Human | PE | A019D5 | 351340 |
| IL-7RA | | | |
| Mouse Anti-Human | PE-Daz | L291H4 | 359420 |
| CCR4 | | | |
| Mouse Anti-Human | PE-Cy7 | J252D4 | 356924 |
| CXCR5 | | | |
| Mouse Anti-Human | PE-Cy5 | BC96 | 302608 |
| CD25 | | | |

CP28 - Nonconventional T Cells

| | | | |
|---|---|---|---|
| Mouse Anti-Human | BB515 | M-T271 | 564643 |
| CD27 | | | |
| Mouse Anti-Human | BB700 | RPA-T8 | 566452 |
| CD8 | | | |
| Mouse Anti-Human | BV421 | 11F2 | 744870 |
| gdTCR | | | |
| Mouse Anti-Human | BV510 | HIB19 | 302242 |
| CD19 | | | |
| Mouse Anti-Human | BV510 | WM15 | 740162 |
| CD13 | | | |
| Mouse Anti-Human | BV605 | OKT3 | 317322 |
| CD3 | | | |
| Mouse Anti-Human | BV650 | 6B11 | 744000 |
| iNKT | | | |
| Mouse Anti-Human | BV711 | B6 | 331412 |
| TCR Vd2 | | | |
| Mouse Anti-Human | BV786 | QA17A04 | 393329 |
| CD57 | | | |
| Mouse Anti-Human | PE | HP-3G10 | 339904 |
| CD161 | | | |
| Mouse Anti-Human | PE-Dazzle594 | 5.1H11 | 362544 |
| CD56 | | | |
| Mouse Anti-Human | PE-Cy7 | 3C10 | 351712 |
| TCR Va7.2 | | | |
| Mouse Anti-Human | PE-Cy5 | HI100 | 304110 |
| CD45RA | | | |

Single Stain Controls

| | | | |
|---|---|---|---|
| ICOS (BB515, BD) | BB515 | DX29 | 337387 |
| CD13 | BB700 | L138 | 1169613 |
| CCR3 | BV421 | 5E8 | B281316 |
| CD19 | BV510 | SJ25C1 | B331406 |
| CD123 | BV605 | 6H6 | B322655 |
| HLA-DR | BV650 | L243 | 307650 |
| CD3 (OKT-3, | BV711 | OKT3 | B317956 |
| Biolegend) | | | |
| CD25 | BV786 | BC96 | B322204 |
| CD66b (Biolegend) | PE | 6/40c | B284868 |
| CD56 | PE-CF594 | 5.1H11 | B325724 |
| | (Dazzle-594) | | |
| CD11c | PE-Cy7 | 3.9 | B308581 |
| CD19 | PE-Cy5 | HIB19 | B311874 |

### RNA Isolation

Isolated WBC for RNA sequencing were centrifuged at 300×g for 5 minutes with a maximum of 1e6 cells per vial. The supernatant was removed, and the cells were resuspended in cold Homogenization Buffer (2% 1-Thioglycerol, Promega). Samples were then frozen at −80° C. until extraction. RNA extraction was performed from frozen samples according to Maxwell RSC simplyRNA Cells Kit (Promega) using the benchtop automated Maxwell RSC Instrument (Promega).

### Library Preparation and Sequencing of Samples

Libraries were prepared with Illumina TruSeq® Stranded mRNA Library Prep (Poly-A mRNA; stranded). Libraries were sequenced on NovaSeq 6000 as Paired-End Reads (2×150) with targeted coverage of 50 mln reads.

### Flow Cytometry Data Processing

Flow cytometry data went through several quality control steps to ensure the consistency and overall high quality of the input in the analysis. All the selected patient samples contained no less than 10 k cells in one panel. Files with poor compensation or occasional PMT failure were excluded. Flow cytometry data was exported in fcs 3.0 file format and analyzed as Pandas DataFrames (v 1.1.4) with compensation matrices applied using FlowKit (v. 0.5.0, https://github.com/malcommac/FlowKit/releases) software for data processing and analysis. The values of all fluorochrome-marker channels were divided by a coefficient of 190 with the following inverse hyperbolic sine: arcsinh x=ln(x+√((x\hat{ }2+1))) transformation. Forward scatter and side scatter values (FCS-A/H/W and SSC-A/H/W) were divided by 105 to meet the order of data transformed with arcsinh.

### Manual Data Analysis

A framework was developed for a precise manual analysis of cell populations combining classical gating within 2D scatter plots and clustering steps. Each panel was analyzed separately in accordance with its own specific strategy. Every strategy consists of several consecutive steps performed of the following cell selection/labeling methods:

Clustering approach. Events were clustered using FlowSOM (v0.1.1, https://pypi.org/project/FlowSom/). Data was visualized with tSNE algorithm (openTSNE, v 0.6.2, https://pypi.org/project/openTSNE/) and coloured both by clustering result and by all markers intensity enabling to see the combination of markers intensities on specific clusters. Each cluster was matched with cell population manually based on a combination of markers intensities on this cluster.

Prior to clustering, processing the cytometry data may include a noise transformation. Noise transformation adjusts the intensity of the markers to reduce the influence of noise on the clustering results and includes reducing the intensity of the marker lower than a certain threshold. Threshold of noise for the marker is defined manually based on a 2-dimensional plot of the intensity of the marker versus intensity of another marker in the panel. The boundary between the noise and positive signal of the marker is chosen at the point of visually observed local minimum of the distribution by markers. Equations below describe the intensity of a marker after the noise transformation:

$${I}_{after transform} = {I}_{inital} , if {I}_{initial} \geq border$$

$${I}_{after transform} = \frac{{I}_{initial}}{k}$$

where I_initial is the initial intensity of the marker from the cytometry data file, border is the threshold of noise for the intensity of the marker, and k is the coefficient of noise reduction. The coefficient of reduction is not a constant, it linearly increases from 1 at the selected threshold of noise to its maximum value (defined as 20) at the minimum intensity of the marker.

Population selection by two-dimensional plot shows pairwise projections of data distribution histograms and colored by distribution density of events (the same as done with classical gating process). The boundary between the positive and negative population is manually chosen at the point visually observed local minimum of the distribution by markers. In order to simplify the visual observation of local minimum of the distribution, kernel density estimate plots are used, above density plot.

The final results of manual data labeling were cell population labels for every event in the fcs file.

### Determination of Cell Percentages

To calculate the final population percentages from labeled data, the results from different cytometry panels were combined together via the general panel (CP10). The cell count values in corresponding populations from other panels were multiplied by normalization coefficients to match results from the linear panel. The normalization coefficient was obtained by dividing the number of cells in the reference population in the linear panel by the number of cells in the reference population in the other panels ((Monocytes for monocytes panel, T cells for CD4 T cells panel, etc.). Table 10 contains the full list of reference populations used to combine results from different panels in order to calculate cell percentages for subpopulations. After this procedure, the percentage of Leukocytes for each cell population was calculated. The final percentages were obtained after multiplying percentages by normalization coefficient calculated in the same way using ratio to number of WBC of three reference populations with hematology analyzer (Monocytes, Lymphocytes and Granulocytes).

TABLE 10
Reference populations used for combining
results from different panels

| | Reference population | Reference population |
|---|---|---|
| Panel | in CP10 | in corresponding panel |
| CP7 | CD3+_T_cells | CD3+_T_cells |
| CP8 | CD3+_T_cells | CD3+_T_cells |
| CP16 | PBMC_cells | PBMC_cells |
| CP22 | CD19+_B_cells | CD19+_B_cells |
| CP23 | Monocytes | Monocytes |
| CP24 | CD3+_T_cells | CD3+_T_cells |
| CP25 | CD3+_T_cells | CD3+_T_cells |
| CP26 | NK_cells | NK_cells |
| CP28 | CD3+_T_cells | CD3+_T_cells |

### RNA-Seq Quality Metrics

Raw FASTQ files quality was analyzed using FastQC (version 0.11.9), FastQ Screen (0.11.1) and MultiQC (version 1.14) software tools. The reference genomes utilized for the creation of BWA aligner indices (for FastQ Screen) included Homo sapiens (GRCh38), Mus musculus, Danio rerio, Drosophila melanogaster, Caenorhabditis elegans, Saccharomyces cerevisiae, Arabidopsis thaliana, Mycoplasma arginini, Escherichia virus phiX174, microbiome (downloaded from NIH Human Microbiome Project website), adapters (provided with FastQC v0.11.9), and UniVec (NCBI). All open source blood RNA-seq type datasets went through the same quality metric procedure as well.

### RNA-Seq Processing

Bulk RNA-seq fastq files were processed by Kallisto, version (PMID: 27043002). The Kallisto index file was downloaded from the Xena project (PMID: 28398314), this index file was built based on GENCODE transcriptome annotation version 23 and the human reference genome GRCh38 with genes from the PAR locus removed (chrY: 10,000-2,781,479 and chrY: 56,887,902-57,217,415) (Vivian et al., 2017). In contrast to paired-end fastq files, single-end fastq files were processed by Kallisto with additional options −1 200-s 15 in line with Xena. Calculated expression results were presented in the TPM format. All open source blood RNA-seq type datasets obtained from GEO or ArrayExpress were processed the same way as internal RNA-seq data. For further details of RNA-seq processing see deconvolution publication (PMID: 35944503).

Cell Deconvolution with Kassandra Algorithm

Kassandra is a cell deconvolution algorithm used for the digital reconstruction of the cellular composition of samples from gene expression data (PMID: 35944503). That is a decision tree machine learning technique trained on artificial mixes made from a broad collection of 9,414 tissue and blood sorted cell RNA seq samples. From profiles of sorted cells 150 000 of artificial transcriptomes were generated to train each cell type model. In each artificial mix, the fractions of all cell types were selected from a Dirichlet distribution with concentration parameters inversely proportional to the number of types. Each model was trained to predict the percent RNA fraction of each cell type represented in the mix using LightGBM version 2.3.1. The proportions predicted by the regressors were rescaled to sum up to 1. RNA seq proportions were recalculated into cell proportions using rna-per-cell coefficients derived from literature data.

### Clusterization Algorithm

Flow cytometry data were represented as cell percentages (from total number of WBC for granulocyte populations and from total number of PBMC percentages for all other populations) see Table 11. Major cell populations (also represented in Kassandra deconvolution method) were selected for the cluster analysis with addition of manually selected ICI-relevant cell populations based on extensive publication analysis: TIGIT+PD1+CD8 T cells (PMID: 33188038), Vdelta2+gamma-delta T cells (PMID: 27400322), CD39+Tregs (PMID: 32117275), HLA-DRlow monocytes (PMID: 26787752, 33842304, 32939320, 26873574, 31592989, 24844912, 24357148).

TABLE 11
Cluster populations and normalization

| Cluster_populations | Normalization |
|---|---|
| CD4_Naive_Tregs | PBMC |
| CD4_Naive_T_cells | PBMC |
| CD8_Naive_T_cells | PBMC |
| Naive_B_cells | PBMC |
| Non-switched_Memory_IgM_B_cells | PBMC |
| gdT_Vdelta2+ | PBMC |
| Class-switched_Memory | PBMC |
| CD8_Central_Memory | PBMC |
| CD4_Tregs | PBMC |
| CD4_Transitional_Memory | PBMC |
| CD4_Central_Memory | PBMC |
| CD4_Memory_T_helpers | PBMC |
| CD4_T_cells | PBMC |
| CD39_CD4_Tregs | PBMC |
| Eosinophils | WBC |
| Basophils | WBC |
| Plasmacytoid_Dendritic_cells | PBMC |
| Dendritic_cells | PBMC |
| TIGIT+_PD1+_CD8_T_cells | PBMC |
| CD8_Transitional_Memory | PBMC |
| Mature_NK_cells | PBMC |
| Immature_NK_cells | PBMC |
| CD8_Memory_T_cells | PBMC |
| CD8_T_cells | PBMC |
| CD4_Effector_Memory | PBMC |
| NKT_cells | PBMC |
| CD8_TEMRA | PBMC |
| CD8_Effector_Memory | PBMC |
| CD4_TEMRA | PBMC |
| Neutrophils | WBC |
| Granulocytes | WBC |
| Classical_Monocytes | PBMC |
| Non-classical_Monocytes | PBMC |
| HLA-DR-low_Monocytes | PBMC |

Prior to clusterization the data was rescaled just as for min-max normalization but with 2nd and 98th percentiles instead of 0 and 1 respectively. All values outside 0-1 range were clipped to the closest value.

### Formula for Normalization

$$scaled value Vx = \frac{Px - Pq 02}{Pq 98 - Pq 02}$$

Spectral clustering approach (scikit-learn version 1.1.2) was selected for clusterization technique as a better performing method. Spectral clustering is more robust and can be more suitable clusterization algorithm for the data where expected clusters form irregular shape [https://pubmed.ncbi.nlm.nih.gov/35652725/] (probably a link should be provided, something like https://ieeexplore.ieee.org/document/6019693).

### Evaluation of Cluster Number

To find the optimal number of clusters it decided to test which decomposition produces the most distinct immunotypes. For this clustering technique with the various number of clusters starting with 2 up to 14 was tested. For each decomposition all possible pairs of subtypes were compared between each other with the Further Mann Whitney U test being applied for each pair of clusters for each feature (34 populations) to check if these clusters statistically differ from each other by this population. Then for p-values from all comparisons (number of features×number of permutations without repetitions) Bonferroni correction has been applied. Finally for each pair of clusters the number of p-values lower than the selected threshold (0.05) was calculated and the median number of those significant p-values in every clustering iteration was found. In Table 12 median number of features which significantly distinguish each pair of clusters for the decompositions with number of clusters from 2 to 14 is presented. It can be noticed that for the decompositions with number of clusters 4 and 5 this median number of features is the same and the highest across all options. Decomposition with 5 clusters was chosen as the highest number of clusters which covers all diversity of data and still produces significantly different groups.

TABLE 12
median number of features per cluster

| Number of clusters | Number of features |
|---|---|
| 2 | 27 |
| 3 | 27 |
| 4 | 28 |
| 5 | 28 |
| 6 | 22 |
| 7 | 24 |
| 8 | 25 |
| 9 | 25 |
| 10 | 23 |
| 11 | 25 |
| 12 | 22 |
| 13 | 23 |
| 14 | 22 |

Optimal cluster number was evaluated for the cohort and found out that clustering with 4 and 5 clusters gives a maximum score of distinct features between each pair of clusters and that score drops with 6 clusters, Therefore spectral clustering was performed with 5 clusters, as 5 clusters was the highest number of clusters which covers maximal observable diversity of the cohort data.

This immunophenotyping assay was evaluated for sensitivity, reproducibility, and repeatability on fresh whole blood. Populations detected in frequencies greater than 0.01% displayed coefficients of variation that were on average less than 10%.

### Differential Expression Analysis

Differential expression (DE) analysis was conducted using the edgeR tool (https://bioconductor.org/packages/release/bioc/html/edgeR.html). Heat shock genes and sex genes were excluded from the analysis.

### Gene Set Enrichment Analysis (GSEA) of Differentially Expressed Genes

GSEA analysis was performed on an unfiltered list of 200 genes, ranked in descending order of differential expression test statistics. The Compute Overlaps tool (https://www.gsea-msigdb.org/gsea/msigdb/help_annotations.jsp#overlap) was used to compare the gene sets with the H gene set (hallmark gene sets) and the CP gene set (canonical pathways) from the MSigDB collection. For each cluster genset, 22 gene sets were chosen in the collections that best overlap with the gene set. These results and chose N signatures were chosen that are most interesting from the point of view of cluster characterization.

Signature values were calculated using ssGSEA, normalized and shown as a heatmap. The ssGSEA score of PD1 related signatures was also calculated for patients on PD1 therapy.

### Pseudotime Analysis

Pseudotime analysis was performed with the usage of Monocle software [PMID: 24658644]. Monocle is an unsupervised algorithm initially developed to perform on a single-cell RNA-seq data to analyze the cell fate decisions based on gene expression data. Since the analysis aimed to analyze the connection not between different cells, but between different blood samples, it was run again on cell percentages obtained from flow cytometry data analysis.

### Cluster-Aligning Multiclass Classifier

The TabPFN multiclass classification model with default parameters was employed to analyze the comprehensive cohort data. The model was trained on the complete dataset, which was labeled with corresponding clusters using a selected list of features. To enhance the model's performance, the Leave-One-Out cross-validation method for model evaluation was utilized.

### Classifier for HNSCC Data

In case of missing some surface cell markers presence in thawed samples, some of cell populations were replaced to those populations that were corresponding parents on the hierarchy tree. After proving that the internal and HNSCC cohorts data have similar distribution using a Kernel Maximum Mean Discrepancy (MMD), a multiclass classification TabPFN model was trained on the initial cohort with the same cross-validation approach. The model achieved a macro average F1-score of 0.84 and a weighted average F1-score of 0.82. As the TabPFN model turned out to be suitable for the cohort, it was applied to the HNSCC dataset to align each sample to the corresponding cluster.

### Results

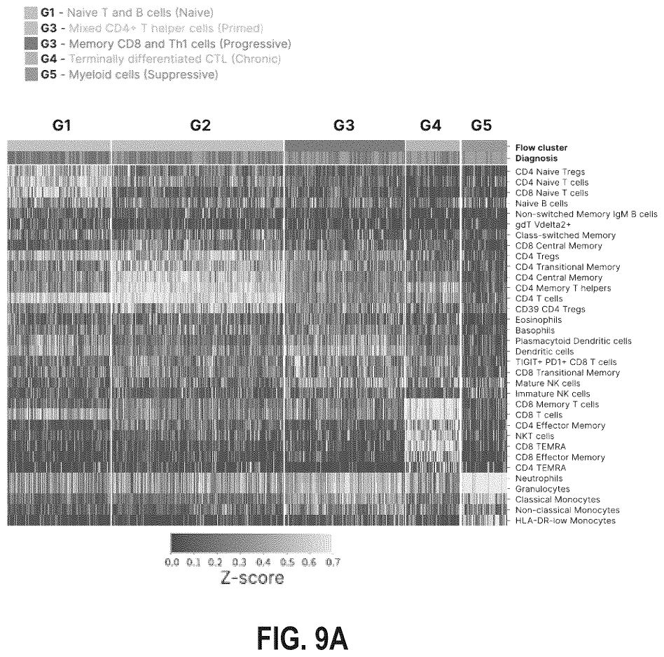

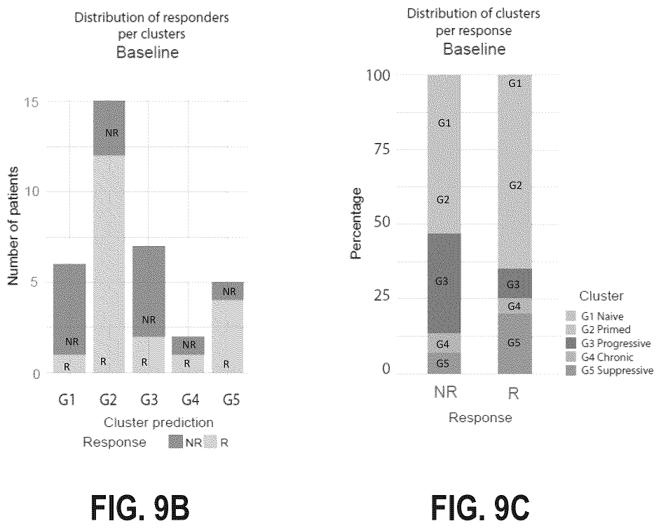

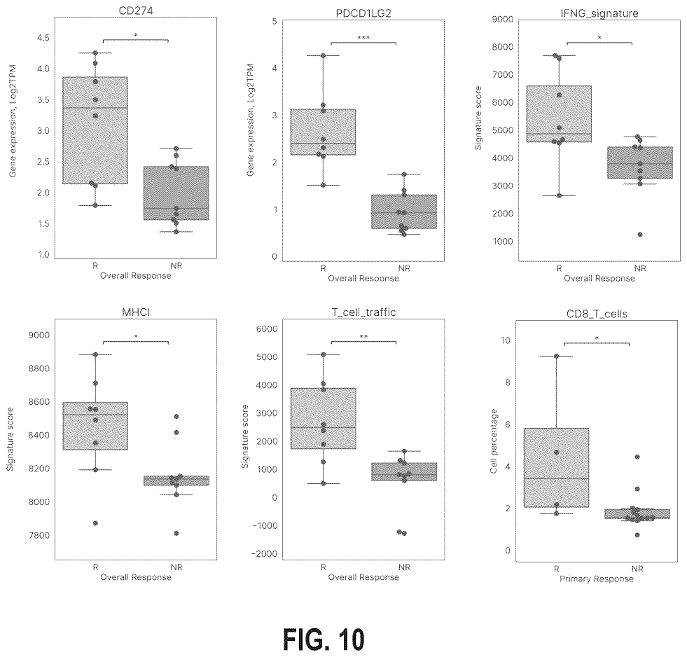

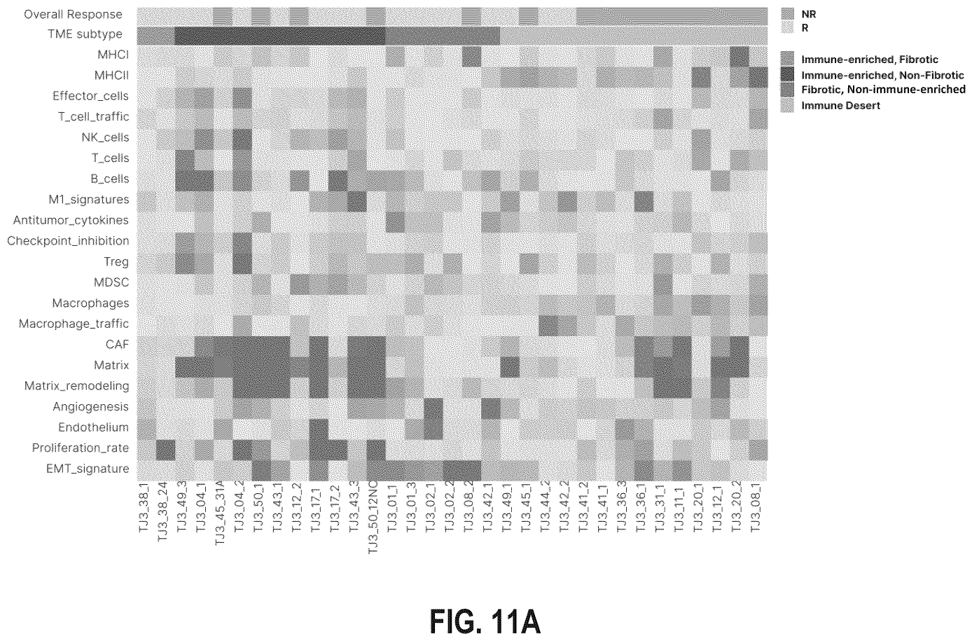

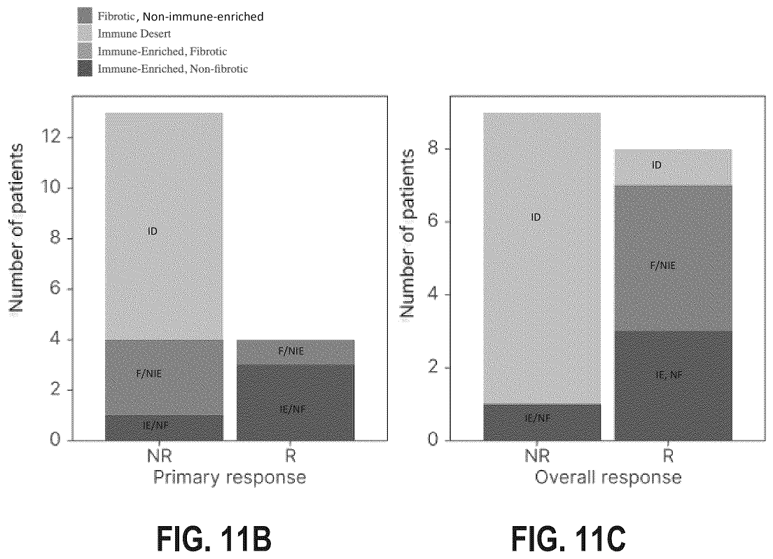

Immunoprofiling of the mixed cohort revealed five conserved immunotypes enriched in certain cell types (G1-naive T and B cells; G2-central memory CD4+ T cells; G3-transitional memory CD8+ T cells; G4-effector memory CD8+ T cells; G5-monocytes/granulocytes) with immunotypes clustering to different disease states in these patients. FIG. 9A is an example showing the segregation of blood samples into the five different immunotypes. HNSCC patients of the clinical cohort treated with nivolumab were stratified into the G1-G5 immunotypes. At baseline, as shown in FIG. 9B and FIG. 9C, the G2 group had higher OR rates than other groups (Fisher's exact test; p=0.02). As shown in FIG. 10, baseline primary tumors showed OR correlated with PD-L1 and PD-L2 expression, interferon responsive genes, T-cell trafficking, and MHC class I pathway (higher values in Responders versus Non-responders, p<0.05). Cell deconvolution showed CD8+ T cell infiltration in the TME correlated with primary site response (p<0.01). As shown in FIG. 11A, while all 12 patients with immune-desert TMEs showed no primary site response (p=0.003), 4/5 patients with an immune-enriched TME showed a primary site response (p=0.002). As shown in FIG. 11B and FIG. 11C, primary tumors with fibrotic TMEs showed no response. However, in patients with a fibrotic TME and a positive OR, indicated by a significant pTR, the G2 immunotype was identified.

### Example 2—Integrated Immunoprofiling and RNA Sequencing (RNA-Seq) for Anti-PD-1 Response Prediction in Head and Neck Squamous Cell Carcinomas

This example shows that immunoprofiling of PBMCs and RNA-seq of tumor tissue can be used to accurately predict response to anti-PD-1 in human papillomavirus negative head and neck squamous cell carcinomas (HPV-HNSCC).

### Methods

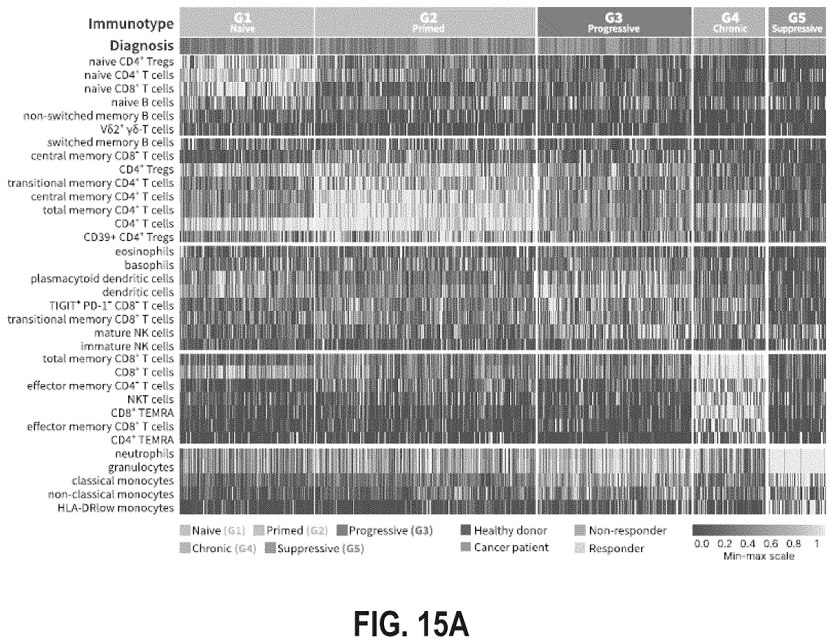

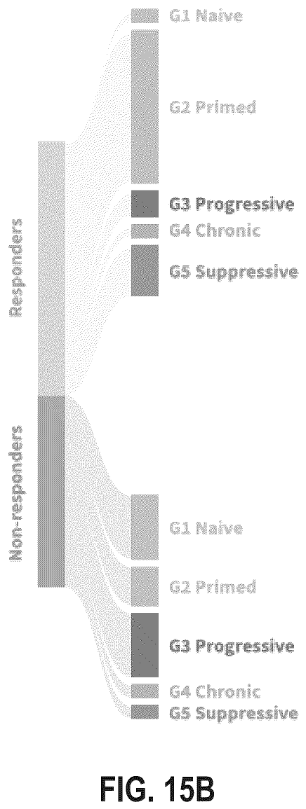

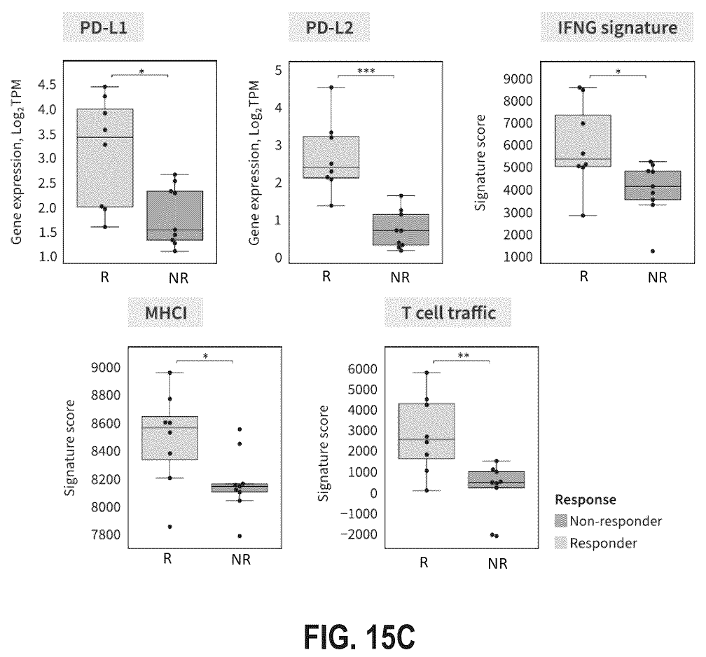

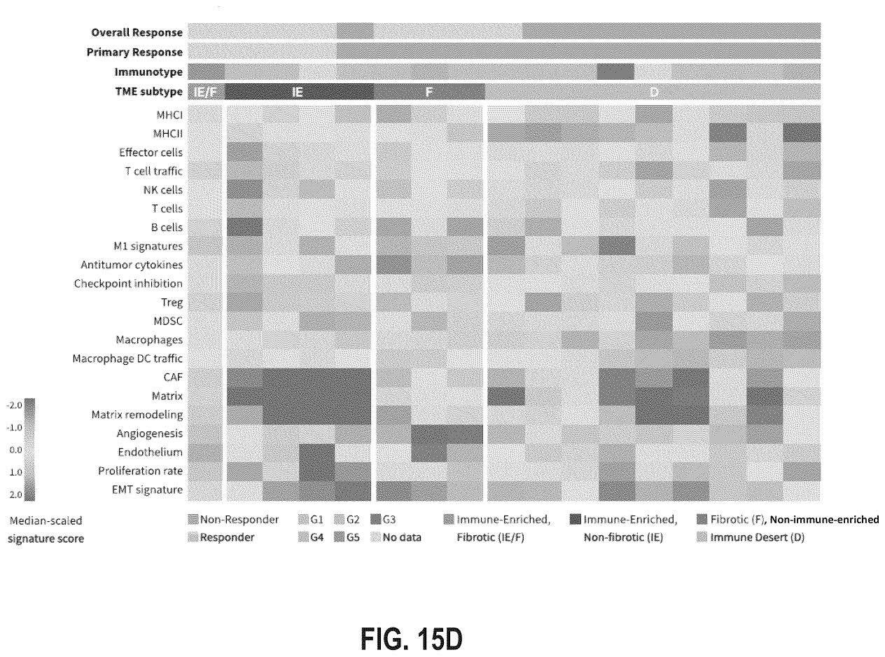

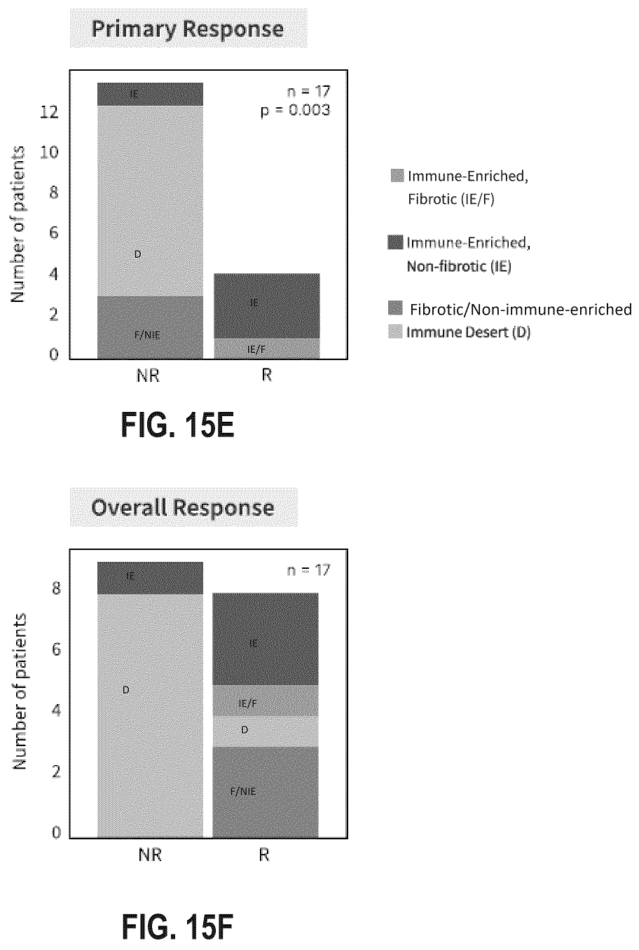

A clinical immunoprofiling platform was developed to characterize the heterogeneity of immune cells in the peripheral blood of healthy donors and patients with solid tumors (n=850). Robust cell populations that were differentially represented in these two groups were selected to train a machine learning (ML)-based classifier and identify groups or immunotypes with putative functional significance. Unsupervised clustering of normalized cell population frequencies for the cell types shown in FIG. 15A from batched flow cytometry data utilizing a common backbone and variable functional staining panels was used to classify patients into five different immunotypes. Populations were then analytically validated by cellular deconvolution of matched RNA-seq data with Kassandra from the same specimens. PBMCs from previously untreated stage II-IV HNSCC patients (n=36) were analyzed at baseline and on-treatment with the anti-PD-1 inhibitor nivolumab+/−an IDO inhibitor. RNA-seq was retrospectively performed on tumors at baseline and on-treatment, along with transcriptome-based tumor microenvironment (TME) subtyping and cellular deconvolution with Kassandra. All disease sites were assigned a pathological treatment response (pTR) and analysis was completed based on primary site response alone and overall response (OR) based on all disease sites. The “Methods” section of Example 1 is applicable to Example 2.

### Results

Blood immunoprofiling of the internal cohort revealed five conserved immunotypes enriched in certain cell types (G1—naïve T and B cells; G2—central memory CD4+ T cells; G3—transitional memory CD8+ T cells; G4—effector memory CD8+ T cells; G5—monocytes/granulocytes), with immunotypes clustering to different disease states in these patients. As shown in FIG. 15A, unsupervised spectral clustering analysis was applied to normalized flow cytometry percentages to reveal five distinct immunotypes based on the distribution of selected cell populations. Samples are also categorized based on patient diagnosis (e.g., healthy donors or cancer patients).

The multi-class immunotype classification mode was used to stratisfy the 36 HNSCC patients treated with nivolumab into the same G1-G5 immunotypes. Among all of the immunotypes, the G2 group had the largest proportion of responders (p=0.02). FIG. 15B shows a Sankey plot showing the distribution of the five immunotypes among responders and non-responders.

Further results of primary tumor analysis were obtained on HPV-negative (HPV-) HNSCC samples. Baseline primary tumors showed OR correlated with PD-L1 and PD-L2 expression, interferon responsive genes, T cell trafficking, and MHC class 1 pathway (higher values in Responders versus Non-responders, p<0.05). FIG. 15C shows box plots representing comparison of pre-treatment samples of responders (R) and non-responders (NR) to nivolumab in the HPV-HNSCC cohort (n=17). The y-axis shows the normalized gene expression value, raw signature score, or cell percentage obtained by the cell deconvolution algorithm Kassandra.

Cell deconvolution showed greater CD8+ T cells in the TME correlated with primary site response (p<0.01) All 9 patients with immune-desert TMEs showed no primary site response (p=0.003); 4/5 patients with an immune-enriched TME showed a primary site response (p=0.002). Patients with a fibrotic TME and G2 immunotype showed overall response at distant sites. None of these associations were discovered on HPV-positive HNSCCs. FIG. 15D shows the transcriptome-based classification of pre-treatment primary tumor samples from HPV-HNSCC cohort (n=17) into four TME subtypes. FIG. 15E and FIG. 15F show, respectively, the association of primary and overall) response to nivolumab with TME subtypes of HPV-HNSCC pre-treatment samples. In FIG. 15E and FIG. 15F, IE stands for immune-enriched, non-fibrotic; E/F stands for immune-enriched, fibrotic; F stands for fibrotic; and D stands for immune desert.

### Conclusion

The results suggest that integrated immunoprofiling has potential as a tool for developing biologic predictors of response to ICI therapies for cancers including HPV-HNSCCs.

### Example 3—Accurate Therapeutic Response Prediction

This example shows that techniques described herein can be used to accurately predict whether a subject will respond to ICI therapy.

In this example, data from subjects in the Thomas Jefferson University (TJU) head and neck squamous cell carcinoma cohort was used to (a) select MF profile types for the subjects, (b) select immunprofile types, and (c) predict whether the subject would respond to nivolimumab. Among 32 subjects, 15 were HPV- and 17 were HPV+.

The MF profile types were selected according to embodiments of the technology described herein for selecting MF profile types such as, for example, the embodiments described with respect to FIG. 1B, FIG. 3A, FIG. 3B, FIG. 4A, FIG. 8A, FIG. 8B, and in the section “Selecting MF Profile Types.” The immunoprofile types were selected according to embodiments of the technology described herein including at least with respect to FIG. 6A, FIG. 6B, FIG. 6C, and in the section “Selecting Immoprofile Types.”

Therapeutic response was predicted based on the MF profile types selected for the subjects, G2 scores determined for the subjects, and expression of PD-L1. The G2 scores were determined according to embodiments of the technology described herein for determining G2 scores such as, for example, the embodiments described with respect to FIG. 1B, FIG. 3A, FIG. 3B, FIG. 4B, FIG. 4C, FIG. 5, and in the section “Immunoprofile Type Scores.” The G2 scores were normalized with respect to the value 8.923467 (maximum value in the TJU cohort). The expression of PD-L1 was determined based on the expression of CD274 from RNA-seq. The expression values were expressed in TPM and were normalized with respect to 25.756554 (maximum value in the TJU cohort). The MF profile types were encoded with 0 for fibrotic/non-immune-enriched and immune desert types, and with 1 for immune-enriched/fibrotic and immune-enriched/non-fibrotic types.

Therapeutic response was predicted according to embodiments of the technology described herein including at least with respect to FIG. 1B, FIG. 3A, and FIG. 3B. In particular, for a particular subject, the normalized G2 score, encoded MF profile type, and normalized value indicating expression of PD-L1 was provided as input to a logistic regression model. The logistic regression model is from the sklearn package (scikit-learn.org/stable/modules/generated/sklearn.linear_model.LogisticRegression.html). Examples of model coefficients are listed in Table 1. The output was the probability of a response to immunotherapy (from 0 to 1), or discrete values of 0 for no response and 1 for response.

FIGS. 12A-12B show the MF profile types determined for responders and non-responders. As shown in FIG. 12A and FIG. 12B, subjects for which immune-enriched MF profile types were selected (e.g., immune-enriched/fibrotic or immune-enriched/non-fibrotic) were more likely to be responsive to nivolimumab than subjects for which a non-immune-enriched MF profile (e.g., fibrotic or immune desert). This is evidenced by the odds ratio of 4.7, which indicates that the subject is more likely to be responsive to nivolimumab to the subject when the tumor sample is of an immune-enriched MF profile type than when tumor sample is of a non-enriched MF profile type.

FIGS. 12C-12D show the immunoprofile types determined for responders and non-responders. As shown in FIG. 12C and FIG. 12D subjects for which the Primed (G2) immunoprofile type was selected were more likely to be responsive to nivolimumab than subjects for which a non-G2 immunoprofile type was selected (e.g., G1, G3, G4, and G5). This is evidenced by the odds ratio of 7.5, which indicates that the subject is more likely to be responsive to nivolimumab to the subject when the tumor sample is of an G2 immunoprofile type than when tumor sample is of a non-G2 immunoprofile type.

FIGS. 12E-12F show that, combined, data derived from blood samples (e.g., immunoprofile type data) and data derived from tumor data (e.g., MF profile type data) increases prediction accuracy. As shown in FIG. 12E and FIG. 12F subjects for which the Primed (G2) immunoprofile type and an immune-enriched MF profile type were selected were more likely to be responsive to nivolimumab than subjects for which a non-G2 immunoprofile type and/or a non-immune-enriched MF profile type was selected. This is evidenced by the odds ratio of 9.9, which indicates that the subject is more likely to be responsive to nivolimumab to the subject when the tumor sample is of an G2 immunoprofile type and an immune-enriched MF profile type than when tumor sample is of a non-G2 immunoprofile type and/or a non-immune-enriched MF profile type. This odds ratio is greater than that observed for the tumor-only data shown in FIGS. 12A-12B and the blood-only data shown in FIG. 12C-12D.

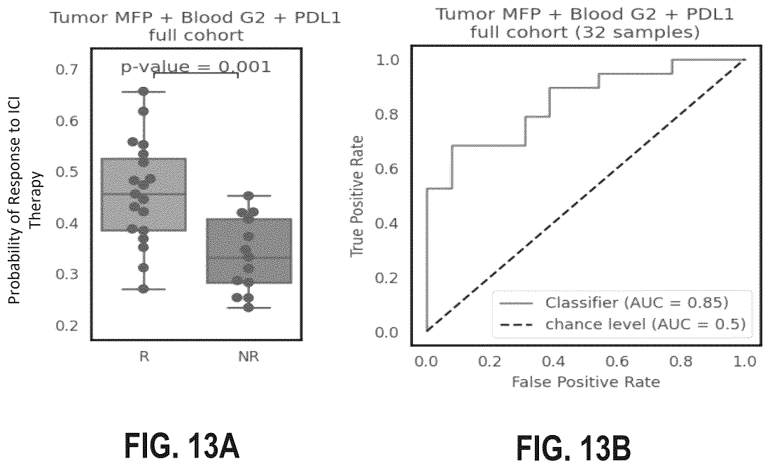

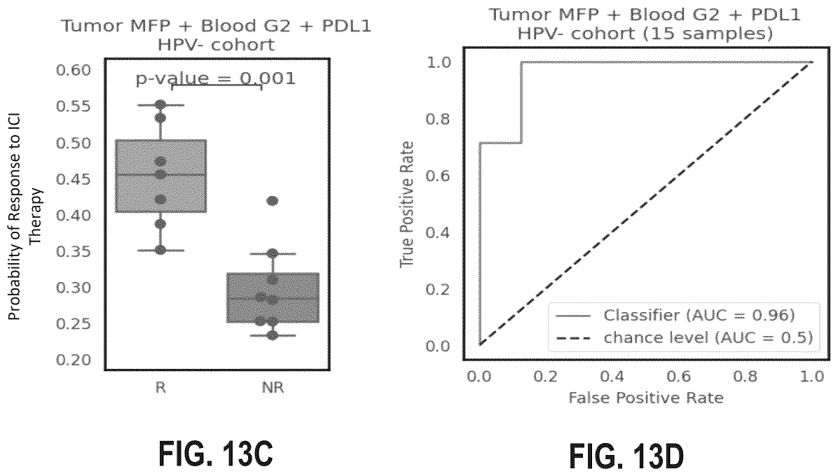

FIG. 13A, FIG. 13B, FIG. 13C and FIG. 13D are results showing that the G2 score, MF profile type, and PD-L1 expression accurately distinguished between subjects who were responsive and subjects who were non-responsive to treatment with nivolimumab.

### G2 Score: Data Preparation and Model Training

The G2 signature can be calculated from blood cell flow cytometry data or from blood cell RNA sequencing data after using RNA-seq-based deconvolution. When using flow cytometry data, cell composition percentages were obtained for the cell types listed in Table 2. When using RNA-seq-based deconvolution, cell composition percentages were obtained for the cell types listed in Table 3.

The training cohort included blood composition percentages from whole blood cells (WBC) of healthy donors and patients with solid tumors (BostonGene internal cohort). For a signature based on flow cytometry, flow cytometry data was used, and for a signature based on deconvolution, RNA sequencing data was used. Labels for immunoprofile types G1-G5 were used for training, G2 is encoded by 1, the rest of the immunoprofile types were encoded by 0.

Cell percentages of all the populations except granulocytes were normalized by the PBMC percentage.

The percentages of immune cell populations for each person from the cohort were normalized using 0.02 (q1) and 0.98 (q2) quantiles of percentages' distribution in the training dataset. (Equation 1).

ElasticNet linear regression was used to identify coefficients that linearly transform percentages of cell populations to score separating the G2 immunoprofile cluster from the other immunoprofile clusters. Normalized percentages of cell populations were used as features, labels of immune portraits in the form of 0 or 1 were used as target values for regression. Model parameters alpha and 11 ratio were selected by grid search. The optimization score for grid search was cross-validated ROC AUC for regression output value differentiating G2 from other clusters. Cross-validation was made with StratifiedShuffleSplit (n_splits=5, test_size=0.3).

The constructed regression model took normalized cellular percentages as input and as an output gave a value approximately in the range covering values from −0.25 to 1.25 (but there may be values below or above these numbers).

After model training, linear regression values (output of the model) were obtained for training cohort samples, and 0.01 (qp1) and 0.99 (qp2) quantiles of cohort predictions were calculated. These values were saved for the normalization of G2 score calculated for new patients to 0.1-10 range. Example coefficients of the linear regression model trained using cytometry data are shown in Table 2. Example coefficients of the linear regression model trained using the RNA-seq data are shown in Table 3.

### Example PBMC Signature Clusters Associated With Immunoprofile Types

Tables 13-18 describe a first set of example PBMC signature clusters. The first set of example PBMC signature clusters were obtained as follows:

Samples from 621 blood draws in total were collected: 299 being from healthy donors, 221 from patients with epithelial cancers, and 101 from sarcoma patients. These samples were subject to the crosslinking multipanel flow cytometry (FC) analysis, as well as a hematology analyzer. For most of the samples, RNA sequencing was also performed. As a result, a cohort with multiple cell populations' percentages in blood (e.g., cell types set forth in Table 5) was generated. For most of the blood samples from cancer patients, corresponding RNA-seq of a tumor biopsy was available. For RNA-seq data, there were expression values calculated in TPM format for approximately 20,000 genes.

At first, flow cytometry data were analyzed using classical dimensional reduction methods, such as PCA, tSNE and uMAP. Cluster map analysis was performed on the data. Different types of clustering algorithms were used: hierarchical (ward), Louvain clustering, Leiden clustering, k-nearest neighbors, HDBscan, and spectral clustering. The performance of these algorithms was evaluated on the data based on the stability of clusters obtained by each method, with bootstrapping the dataset. The best stability of clusters was observed with the spectral clustering algorithm with the number of clusters being equal to 5.

The clusters may be described statistically, as shown in Tables 13-15 below, which show, the 25%, 50% (median), and 75% quantiles for each of the five clusters for each of the cell types.

TABLE 13

| | | G2 | G3 | G4 | G5 |
|---|---|---|---|---|---|
| | G1 | (CD8 | (CD4 | (CD4/CD8 | (Naïve |
| | (Monocytes) | T cells) | T cells) | T cells) | T/B cells) |
| | 25% | 25% | 25% | 25% | 25% |
| HLA-DR-T cells | 0.106328 | 0.283683 | 0.5403 | 0.443324 | 0.596917 |
| CD4 T cells | 0.067698 | 0.267173 | 0.58441 | 0.374826 | 0.511845 |
| Th1 CD4 T cells | 0.036604 | 0.108995 | 0.26649 | 0.263428 | 0.152247 |
| Th2 CD4 T cells | 0.095003 | 0.142166 | 0.37654 | 0.185264 | 0.163 |
| Th17 CD4 T cells | 0.117065 | 0.153179 | 0.42137 | 0.230167 | 0.226913 |
| CD4 Naïve T cells | 0.034715 | 0.063445 | 0.23172 | 0.17332 | 0.439375 |
| CD4 Naïve Tregs | 0.032717 | 0.059123 | 0.19235 | 0.131932 | 0.289133 |
| CD4 Memory T helpers | 0.063865 | 0.375559 | 0.61633 | 0.382133 | 0.242293 |
| CD4 Effector Memory | 0.058556 | 0.456092 | 0.20736 | 0.229745 | 0.122493 |
| CD4 Central Memory | 0.123691 | 0.246346 | 0.60517 | 0.335551 | 0.291734 |
| CD4 TEMRA | 0.005659 | 0.11697 | 0.01225 | 0.013615 | 0.010809 |
| CD8 T cells | 0.039958 | 0.630845 | 0.19631 | 0.331358 | 0.295225 |
| CD8 Naïve T cells | 0.020406 | 0.046728 | 0.08689 | 0.108112 | 0.247128 |
| CD8 Memory T cells | 0.061191 | 0.659571 | 0.19163 | 0.284277 | 0.166347 |
| CD8 Transitional | 0.036002 | 0.093951 | 0.20409 | 0.313713 | 0.174481 |
| Memory PD-1+ | | | | | |
| CD8 Transitional | 0.058921 | 0.231584 | 0.23021 | 0.340671 | 0.190939 |
| Memory | | | | | |
| CD8 Central Memory | 0.036013 | 0.128753 | 0.20134 | 0.231671 | 0.160843 |
| CD8 Effector Memory | 0.010973 | 0.152987 | 0.05532 | 0.096939 | 0.041693 |
| Follicular T cells | 0.074380 | 0.192826 | 0.45744 | 0.326113 | 0.269109 |
| CD8 TEMRA | 0.009394 | 0.452182 | 0.02534 | 0.036895 | 0.02272 |
| CD8 TEMRA PD-1+ | 0.003883 | 0.069517 | 0.03595 | 0.052409 | 0.033727 |
| Non-switched Memory | 0.035967 | 0.026193 | 0.07142 | 0.101036 | 0.145749 |
| IgM B cells | | | | | |
| Class-switched Memory | 0.042619 | 0.050361 | 0.10672 | 0.133283 | 0.13904 |
| Naïve B cells | 0.093303 | 0.062922 | 0.11898 | 0.14919 | 0.221864 |
| Classical Monocytes | 0.458568 | 0.244505 | 0.24327 | 0.248523 | 0.232693 |
| Non-classical | 0.230355 | 0.156849 | 0.10795 | 0.143944 | 0.069608 |
| Monocytes | | | | | |
| Mature NK cells | 0.200234 | 0.087801 | 0.18181 | 0.180878 | 0.103363 |
| Immature NK cells | 0.139125 | 0.058377 | 0.12202 | 0.116228 | 0.091874 |
| Dendritic cells | 0.106136 | 0.086082 | 0.15349 | 0.216583 | 0.160171 |
| Plasmacytoid Dendritic | 0.162407 | 0.128447 | 0.19228 | 0.236386 | 0.218051 |
| cells | | | | | |
| cDC2 | 0.090028 | 0.104803 | 0.1491 | 0.260122 | 0.169601 |
| NKT cells | 0.022841 | 0.359754 | 0.05384 | 0.090443 | 0.054794 |
| Basophils | 0.087037 | 0.133056 | 0.18685 | 0.203315 | 0.153611 |
| Eosinophils | 0.045604 | 0.036612 | 0.07923 | 0.108351 | 0.073041 |
| Neutrophils | 0.65833 | 0.368159 | 0.38571 | 0.26889 | 0.346164 |
| Granulocytes | 0.666864 | 0.353082 | 0.36287 | 0.239295 | 0.324922 |

TABLE 14

| | | G2 | G3 | G4 | G5 |
|---|---|---|---|---|---|
| | G1 | (CD8 | (CD4 | (CD4/CD8 | (Naïve |
| | (Monocytes) | T cells) | T cells) | T cells) | T/B cells) |
| | Median | Median | Median | Median | Median |
| HLA-DR-Tcells | 0.268192 | 0.500976 | 0.648245 | 0.591865 | 0.758814 |
| CD4 T cells | 0.209573 | 0.343746 | 0.68407 | 0.486548 | 0.651823 |
| Th1 CD4 T cells | 0.104613 | 0.204741 | 0.402059 | 0.396065 | 0.291833 |
| Th2 CD4 T cells | 0.147361 | 0.214283 | 0.479981 | 0.244601 | 0.25253 |
| Th17 CD4 T cells | 0.217881 | 0.272969 | 0.552144 | 0.332609 | 0.312792 |
| CD4 Naïve T cells | 0.113725 | 0.125276 | 0.351845 | 0.278363 | 0.600005 |
| CD4 Naïve Tregs | 0.099969 | 0.100974 | 0.293101 | 0.216797 | 0.426822 |
| CD4 Memory T helpers | 0.195943 | 0.466783 | 0.709288 | 0.486498 | 0.375563 |
| CD4 Effector Memory | 0.139278 | 0.61348 | 0.320537 | 0.310928 | 0.198333 |
| CD4 Central Memory | 0.206858 | 0.310895 | 0.688435 | 0.422796 | 0.396236 |
| CD4 TEMRA | 0.016302 | 0.246496 | 0.031503 | 0.035689 | 0.028403 |
| CD8 T cells | 0.158047 | 0.75256 | 0.312818 | 0.482671 | 0.408883 |
| CD8 Naïve T cells | 0.061372 | 0.098375 | 0.171853 | 0.266962 | 0.480484 |
| CD8 Memory T cells | 0.143213 | 0.795461 | 0.275231 | 0.390962 | 0.212354 |
| CD8 Transitional | 0.108799 | 0.175897 | 0.302216 | 0.478071 | 0.277381 |
| Memory PD-1+ | | | | | |
| CD8 Transitional | 0.151472 | 0.385116 | 0.318796 | 0.534504 | 0.289327 |
| Memory | | | | | |
| CD8 Central Memory | 0.082439 | 0.201114 | 0.31736 | 0.318617 | 0.248858 |
| CD8 Effector Memory | 0.049550 | 0.401219 | 0.095028 | 0.148906 | 0.073248 |
| Follicular T cells | 0.166301 | 0.283402 | 0.654542 | 0.418471 | 0.398618 |
| CD8 TEMRA | 0.043413 | 0.668858 | 0.069213 | 0.122074 | 0.056611 |
| CD8 TEMRA PD-1+ | 0.033842 | 0.221638 | 0.072429 | 0.112941 | 0.071543 |
| Non-switched Memory | 0.091232 | 0.071776 | 0.151122 | 0.199213 | 0.234955 |
| IgM B cells | | | | | |
| Class-switched Memory | 0.114907 | 0.128994 | 0.212951 | 0.237468 | 0.24639 |
| Naïve B cells | 0.213448 | 0.192368 | 0.262771 | 0.272111 | 0.388314 |
| Classical Monocytes | 0.655329 | 0.306156 | 0.342229 | 0.368947 | 0.294646 |
| Non-classical | 0.389927 | 0.263495 | 0.202277 | 0.249826 | 0.128144 |
| Monocytes | | | | | |
| Mature NK cells | 0.371677 | 0.235421 | 0.265459 | 0.317649 | 0.160494 |
| Immature NK cells | 0.240692 | 0.113474 | 0.218086 | 0.201711 | 0.135861 |
| Dendritic cells | 0.205308 | 0.185447 | 0.243487 | 0.326594 | 0.23602 |
| Plasmacytoid Dendritic | 0.321997 | 0.20106 | 0.275889 | 0.384948 | 0.296866 |
| cells | | | | | |
| cDC2 | 0.206457 | 0.187459 | 0.270574 | 0.383004 | 0.251595 |
| NKT cells | 0.119644 | 0.441513 | 0.115239 | 0.173497 | 0.103974 |
| Basophils | 0.186265 | 0.235332 | 0.26546 | 0.294347 | 0.249749 |
| Eosinophils | 0.108559 | 0.111721 | 0.163279 | 0.196355 | 0.161185 |
| Neutrophils | 0.764971 | 0.571629 | 0.517067 | 0.401948 | 0.456711 |
| Granulocytes | 0.792903 | 0.553052 | 0.521678 | 0.395461 | 0.438848 |

TABLE 15

| | | G2 | G3 | G4 | G5 |
|---|---|---|---|---|---|
| | G1 | (CD8 | (CD4 | (CD4/CD8 | (Naïve |
| | (Monocytes) | T cells) | T cells) | T cells) | T/B cells) |
| | 75% | 75% | 75% | 75% | 75% |
| HLA-DR-Tcells | 0.410735 | 0.690284 | 0.797729 | 0.716651 | 0.878565 |
| CD4 T cells | 0.312610 | 0.499894 | 0.769481 | 0.601961 | 0.780222 |
| Th1 CD4 T cells | 0.206641 | 0.288377 | 0.577651 | 0.556095 | 0.445013 |
| Th2 CD4 T cells | 0.231139 | 0.321658 | 0.659031 | 0.342858 | 0.348253 |
| Th17 CD4 T cells | 0.300610 | 0.394415 | 0.736429 | 0.467016 | 0.397839 |
| CD4 Naïve T cells | 0.287645 | 0.252189 | 0.457705 | 0.415348 | 0.748118 |
| CD4 Naïve Tregs | 0.194028 | 0.172061 | 0.434128 | 0.322284 | 0.672625 |
| CD4 Memory T helpers | 0.303711 | 0.658291 | 0.825779 | 0.616362 | 0.515244 |
| CD4 Effector Memory | 0.264000 | 0.767647 | 0.467317 | 0.476475 | 0.296753 |
| CD4 Central Memory | 0.304972 | 0.399709 | 0.837447 | 0.558918 | 0.509379 |
| CD4 TEMRA | 0.065253 | 0.755316 | 0.16683 | 0.113046 | 0.098802 |
| CD8 T cells | 0.249499 | 0.953605 | 0.412224 | 0.619756 | 0.551059 |
| CD8 Naïve T cells | 0.138573 | 0.166204 | 0.302362 | 0.420946 | 0.734749 |
| CD8 Memory T cells | 0.239187 | 0.952402 | 0.370278 | 0.566457 | 0.310643 |
| CD8 Transitional | 0.291578 | 0.375086 | 0.426108 | 0.684213 | 0.401351 |
| Memory PD-1+ | | | | | |
| CD8 Transitional | 0.357580 | 0.538933 | 0.412406 | 0.731612 | 0.408541 |
| Memory | | | | | |
| CD8 Central Memory | 0.166129 | 0.328062 | 0.459847 | 0.489201 | 0.344726 |
| CD8 Effector Memory | 0.106249 | 0.86332 | 0.168937 | 0.247834 | 0.125704 |
| Follicular T cells | 0.274724 | 0.379509 | 0.821956 | 0.593423 | 0.509134 |
| CD8 TEMRA | 0.135954 | 0.973296 | 0.171905 | 0.276018 | 0.126378 |
| CD8 TEMRA PD-1+ | 0.111959 | 0.384143 | 0.140749 | 0.249695 | 0.167744 |
| Non-switched Memory | 0.161719 | 0.135083 | 0.269189 | 0.323938 | 0.378774 |
| IgM B cells | | | | | |
| Class-switched Memory | 0.208408 | 0.189463 | 0.420818 | 0.453476 | 0.417536 |
| Naïve B cells | 0.315093 | 0.300502 | 0.386124 | 0.377916 | 0.565337 |
| Classical Monocytes | 0.829032 | 0.492836 | 0.463403 | 0.490062 | 0.379943 |
| Non-classical | 0.636081 | 0.385348 | 0.318401 | 0.414433 | 0.224774 |
| Monocytes | | | | | |
| Mature NK cells | 0.554361 | 0.479733 | 0.425762 | 0.529321 | 0.265483 |
| Immature NK cells | 0.385627 | 0.237602 | 0.355379 | 0.294723 | 0.228888 |
| Dendritic cells | 0.363509 | 0.258012 | 0.307069 | 0.466837 | 0.33715 |
| Plasmacytoid Dendritic | 0.524641 | 0.298336 | 0.408698 | 0.57627 | 0.471078 |
| cells | | | | | |
| cDC2 | 0.408302 | 0.287179 | 0.376809 | 0.540043 | 0.384745 |
| NKT cells | 0.218663 | 0.775325 | 0.255804 | 0.322626 | 0.189051 |
| Basophils | 0.298585 | 0.301003 | 0.391303 | 0.448906 | 0.330341 |
| Eosinophils | 0.214113 | 0.257368 | 0.272007 | 0.355815 | 0.267526 |
| Neutrophils | 0.865122 | 0.728919 | 0.654019 | 0.529779 | 0.616451 |
| Granulocytes | 0.911527 | 0.73119 | 0.653567 | 0.543972 | 0.612533 |

Second, Tables 16-18 describe a second set of example PBMC signature clusters. The second set of example PBMC signature clusters were obtained as follows:

Peripheral blood samples of 442 cancer patients with differing diagnoses and of 408 healthy donors were collected from multiple centers. White blood cells (WBC) were isolated, stained with custom antibody panels in 96-well plates, and processed by multiparameter flow cytometry (n=850). Each panel was labeled manually to then determine the percentages of cell populations (e.g., cell types set forth in Table 6). A machine-learning model was developed to classify healthy and cancer groups, and refined to stratify immune profiles.

Supervised manual gating of flow cytometry data from a cohort of 50 healthy donors identified 415 cell types and immune activation states that were used to train and independently validate machine learning (ML) models to automatically identify immune cell subsets from raw cytometry data. Using the Boruta feature selection algorithm (see e.g., M Kursa and W. Rudnicki, “Feature Selection with the Boruta Package”, Journal of Statistical Software, vol. 36, issue 11, 2010), 78 significant features were selected from the flow cytometry data. The Random Forest model was further refined using spectral clustering with bootstrapping to identify immune profiles, and cluster stability was measured with Jaccard Index metrics.

The developed machine-learning classification model can differentiate between healthy individuals and cancer patients from flow cytometry analysis of peripheral blood samples. Immune cell heterogeneity in the peripheral blood of individuals was grouped into five (5) PBMC immunoprofile types, each characterized by specific physiological immune programs and supported by transcriptomic analysis.

The clusters may be described statistically, as shown in Tables 16-18 below, which show, the 25%, 50% (median), and 75% quantiles for each of the five clusters for each of the cell types.

TABLE 16

| | G1 | G2 | G3 | G4 | G5 |
|---|---|---|---|---|---|
| | (Naïve) | (Primed) | (Progressive) | (Chronic) | (Suppressive) |
| | 25% | 25% | 25% | 25% | 25% |
| CD4 T cells | 0.516809 | 0.587592 | 0.27119 | 0.261428 | 0.077586 |
| CD4 Naïve T cells | 0.461229 | 0.225648 | 0.122336 | 0.055161 | 0.063215 |
| CD4 Naïve Tregs | 0.315621 | 0.167623 | 0.094701 | 0.052802 | 0.021256 |
| CD4 Memory T | 0.240765 | 0.547057 | 0.262 | 0.330802 | 0.07266 |
| helpers | | | | | |
| CD4 Effector | 0.053214 | 0.131542 | 0.087894 | 0.287602 | 0.049874 |
| Memory | | | | | |
| CD4 Central | 0.246429 | 0.484443 | 0.221505 | 0.188642 | 0.064234 |
| Memory | | | | | |
| CD4 TEMRA | 0.014031 | 0.021267 | 0.010106 | 0.078115 | 0.011735 |
| CD8 T cells | 0.328793 | 0.223175 | 0.182001 | 0.583353 | 0.062078 |
| CD8 Naïve T cells | 0.384364 | 0.086982 | 0.075898 | 0.042453 | 0.044037 |
| CD8 Memory T | 0.13253 | 0.19558 | 0.170497 | 0.630207 | 0.054764 |
| cells | | | | | |
| CD8 Transitional | 0.138353 | 0.205539 | 0.214316 | 0.191516 | 0.051869 |
| Memory | | | | | |
| CD8 Central | 0.107956 | 0.175376 | 0.124022 | 0.122984 | 0.030678 |
| Memory | | | | | |
| CD8 Effector | 0.044591 | 0.064165 | 0.06174 | 0.205876 | 0.02561 |
| Memory | | | | | |
| CD8 TEMRA | 0.030362 | 0.033965 | 0.032092 | 0.45737 | 0.019798 |
| Non-switched | 0.124961 | 0.083798 | 0.040423 | 0.020677 | 0.021477 |
| Memory IgM B | | | | | |
| cells | | | | | |
| Class-switched | 0.145021 | 0.161367 | 0.071409 | 0.054709 | 0.065685 |
| Memory | | | | | |
| Naïve B cells | 0.230684 | 0.187741 | 0.125653 | 0.072578 | 0.103146 |
| Classical | 0.149244 | 0.1827 | 0.320377 | 0.154462 | 0.391395 |
| Monocytes | | | | | |
| Non-classical | 0.093421 | 0.125546 | 0.220434 | 0.132087 | 0.122624 |
| Monocytes | | | | | |
| Mature NK cells | 0.099844 | 0.142419 | 0.222068 | 0.162549 | 0.144145 |
| Immature NK cells | 0.10621 | 0.09467 | 0.1418 | 0.072917 | 0.075758 |
| Dendritic cells | 0.320098 | 0.220471 | 0.32289 | 0.183333 | 0.039343 |
| Plasmacytoid | 0.24047 | 0.157613 | 0.221469 | 0.126741 | 0.033319 |
| Dendritic cells | | | | | |
| NKT cells | 0.083531 | 0.076961 | 0.073639 | 0.387684 | 0.04147 |
| Granulocytes | 0.247181 | 0.303666 | 0.429831 | 0.239702 | 0.789608 |
| Neutrophils | 0.240015 | 0.310561 | 0.398834 | 0.25917 | 0.771303 |
| Basophils | 0.177694 | 0.170987 | 0.214673 | 0.165205 | 0.044676 |
| Eosinophils | 0.106514 | 0.113121 | 0.139433 | 0.085996 | 0.005973 |
| CD4 Tregs | 0.367483 | 0.377588 | 0.244801 | 0.119928 | 0.053491 |
| CD4 Transitional | 0.191683 | 0.352033 | 0.229838 | 0.160369 | 0.051402 |
| Memory | | | | | |
| HLA DR low | 0.02022 | 0.03144 | 0.049407 | 0.023268 | 0.23573 |
| Monocytes | | | | | |
| TIGIT+ PD1+ | 0.157494 | 0.207882 | 0.207871 | 0.186848 | 0.072178 |
| CD8 T cells | | | | | |
| CD39 CD4 Tregs | 0.220702 | 0.315876 | 0.194143 | 0.133377 | 0.124994 |
| gdT Vdelta2+ | 0.064997 | 0.034592 | 0.034595 | 0.022619 | 0.016564 |

TABLE 17

| | G1 | G2 | G3 | G4 | G5 |
|---|---|---|---|---|---|
| | (Naïve) | (Primed) | (Progressive) | (Chronic) | (Suppressive) |
| | Median | Median | Median | Median | Median |
| CD4 T cells | 0.662711 | 0.685517 | 0.366451 | 0.413697 | 0.177509 |
| CD4 Naïve T cells | 0.556319 | 0.35091 | 0.224878 | 0.13328 | 0.130569 |
| CD4 Naïve Tregs | 0.501201 | 0.266506 | 0.190085 | 0.119554 | 0.075814 |
| CD4 Memory T | 0.362402 | 0.648877 | 0.349596 | 0.488784 | 0.184368 |
| helpers | | | | | |
| CD4 Effector | 0.124962 | 0.243893 | 0.161168 | 0.460197 | 0.12102 |
| Memory | | | | | |
| CD4 Central | 0.335085 | 0.603169 | 0.323676 | 0.289721 | 0.147204 |
| Memory | | | | | |
| CD4 TEMRA | 0.040028 | 0.048572 | 0.02456 | 0.208867 | 0.034468 |
| CD8 T cells | 0.467289 | 0.302725 | 0.332053 | 0.696135 | 0.136891 |
| CD8 Naïve T cells | 0.577479 | 0.184101 | 0.182589 | 0.092848 | 0.085994 |
| CD8 Memory T | 0.212876 | 0.288699 | 0.276308 | 0.753472 | 0.147294 |
| cells | | | | | |
| CD8 Transitional | 0.256088 | 0.313295 | 0.340113 | 0.295304 | 0.135786 |
| Memory | | | | | |
| CD8 Central | 0.174808 | 0.296935 | 0.211254 | 0.204562 | 0.083402 |
| Memory | | | | | |
| CD8 Effector | 0.08312 | 0.108541 | 0.126585 | 0.463977 | 0.071121 |
| Memory | | | | | |
| CD8 TEMRA | 0.075175 | 0.09858 | 0.073416 | 0.6324 | 0.079485 |
| Non-switched | 0.195806 | 0.166557 | 0.125041 | 0.070817 | 0.056502 |
| Memory IgM B | | | | | |
| cells | | | | | |
| Class-switched | 0.256662 | 0.269578 | 0.173303 | 0.131577 | 0.135593 |
| Memory | | | | | |
| Naïve B cells | 0.370449 | 0.298478 | 0.245035 | 0.213552 | 0.163953 |
| Classical | 0.225279 | 0.252791 | 0.41498 | 0.269292 | 0.615564 |
| Monocytes | | | | | |
| Non-classical | 0.144156 | 0.204433 | 0.31591 | 0.238835 | 0.279489 |
| Monocytes | | | | | |
| Mature NK cells | 0.176443 | 0.233585 | 0.401355 | 0.254386 | 0.301891 |
| Immature NK cells | 0.17347 | 0.167108 | 0.22399 | 0.157168 | 0.186185 |
| Dendritic cells | 0.437941 | 0.330493 | 0.480443 | 0.316261 | 0.157078 |
| Plasmacytoid | 0.353953 | 0.254119 | 0.378514 | 0.234899 | 0.121252 |
| Dendritic cells | | | | | |
| NKT cells | 0.171432 | 0.175771 | 0.129615 | 0.539552 | 0.129261 |
| Granulocytes | 0.382618 | 0.449489 | 0.561927 | 0.4229 | 0.850685 |
| Neutrophils | 0.387406 | 0.433641 | 0.529458 | 0.40581 | 0.830025 |
| Basophils | 0.262712 | 0.270411 | 0.301113 | 0.248018 | 0.112651 |
| Eosinophils | 0.20403 | 0.215491 | 0.242424 | 0.192835 | 0.066675 |
| CD4 Tregs | 0.492833 | 0.525276 | 0.366742 | 0.218896 | 0.163226 |
| CD4 Transitional | 0.298263 | 0.497258 | 0.321826 | 0.255247 | 0.151531 |
| Memory | | | | | |
| HLA DR low | 0.065281 | 0.07846 | 0.125353 | 0.067239 | 0.477165 |
| Monocytes | | | | | |
| TIGIT+ PD1+ | 0.240903 | 0.306068 | 0.333351 | 0.342234 | 0.148581 |
| CD8 T cells | | | | | |
| CD39 CD4 Tregs | 0.371016 | 0.520242 | 0.372921 | 0.296799 | 0.200762 |
| gdT Vdelta2+ | 0.140606 | 0.083826 | 0.088897 | 0.054894 | 0.050277 |

TABLE 18

| | G1 | G2 | G3 | G4 | G5 |
|---|---|---|---|---|---|
| | (Naïve) | (Primed) | (Progressive) | (Chronic) | (Suppressive) |
| | 75% | 75% | 75% | 75% | 75% |
| CD4 T cells | 0.788032 | 0.786622 | 0.463021 | 0.564684 | 0.366608 |
| CD4 Naïve T cells | 0.741686 | 0.461062 | 0.327475 | 0.260051 | 0.375022 |
| CD4 Naïve Tregs | 0.764426 | 0.408182 | 0.288943 | 0.241674 | 0.185199 |
| CD4 Memory T | 0.465098 | 0.761053 | 0.45632 | 0.655063 | 0.295012 |
| helpers | | | | | |
| CD4 Effector | 0.208899 | 0.378299 | 0.251368 | 0.772331 | 0.27798 |
| Memory | | | | | |
| CD4 Central | 0.466527 | 0.746517 | 0.437682 | 0.411724 | 0.223683 |
| Memory | | | | | |
| CD4 TEMRA | 0.131756 | 0.220494 | 0.058863 | 0.639782 | 0.143112 |
| CD8 T cells | 0.589538 | 0.468197 | 0.48603 | 0.904461 | 0.352648 |
| CD8 Naïve T cells | 0.78544 | 0.323442 | 0.319262 | 0.170799 | 0.192921 |
| CD8 Memory T | 0.320078 | 0.409544 | 0.455537 | 0.915129 | 0.286708 |
| cells | | | | | |
| CD8 Transitional | 0.415027 | 0.441222 | 0.5326 | 0.450074 | 0.244854 |
| Memory | | | | | |
| CD8 Central | 0.263697 | 0.444168 | 0.354911 | 0.280339 | 0.16354 |
| Memory | | | | | |
| CD8 Effector | 0.160068 | 0.198665 | 0.227355 | 0.809943 | 0.127967 |
| Memory | | | | | |
| CD8 TEMRA | 0.176336 | 0.230469 | 0.21788 | 0.907577 | 0.221438 |
| Non-switched | 0.307385 | 0.267483 | 0.227953 | 0.149117 | 0.14205 |
| Memory IgM B | | | | | |
| cells | | | | | |
| Class-switched | 0.42331 | 0.464562 | 0.289808 | 0.246844 | 0.248892 |
| Memory | | | | | |
| Naïve B cells | 0.571189 | 0.43868 | 0.406178 | 0.360336 | 0.335013 |
| Classical | 0.303541 | 0.362069 | 0.559735 | 0.365677 | 0.863046 |
| Monocytes | | | | | |
| Non-classical | 0.252922 | 0.299484 | 0.495174 | 0.390367 | 0.575074 |
| Monocytes | | | | | |
| Mature NK cells | 0.326604 | 0.380774 | 0.617431 | 0.501855 | 0.440678 |
| Immature NK cells | 0.26615 | 0.274601 | 0.364978 | 0.262244 | 0.360084 |
| Dendritic cells | 0.551467 | 0.424051 | 0.646407 | 0.434698 | 0.306728 |
| Plasmacytoid | 0.521473 | 0.349518 | 0.568588 | 0.380915 | 0.2793 |
| Dendritic cells | | | | | |
| NKT cells | 0.264899 | 0.343661 | 0.256366 | 0.866944 | 0.344584 |
| Granulocytes | 0.517676 | 0.595496 | 0.685545 | 0.57367 | 0.991513 |
| Neutrophils | 0.52906 | 0.572942 | 0.656846 | 0.579071 | 0.988562 |
| Basophils | 0.396037 | 0.432851 | 0.431055 | 0.353198 | 0.207963 |
| Eosinophils | 0.333206 | 0.366476 | 0.423774 | 0.333555 | 0.155757 |
| CD4 Tregs | 0.663132 | 0.668141 | 0.478286 | 0.394052 | 0.306773 |
| CD4 Transitional | 0.453336 | 0.648222 | 0.476315 | 0.372905 | 0.282151 |
| Memory | | | | | |
| HLA DR low | 0.140713 | 0.16051 | 0.304512 | 0.217529 | 0.882418 |
| Monocytes | | | | | |
| TIGIT+ PD1+ | 0.351118 | 0.425046 | 0.545905 | 0.51332 | 0.273978 |
| CD8 T cells | | | | | |
| CD39 CD4 Tregs | 0.483579 | 0.682502 | 0.489588 | 0.389691 | 0.367768 |
| gdT Vdelta2+ | 0.28449 | 0.186779 | 0.200473 | 0.103883 | 0.126101 |

### Computer Implementation

An illustrative implementation of a computer system 1400 that may be used in connection with any of the embodiments of the technology described herein (e.g., such as the process 300 of FIG. 3A, process 350 of FIG. 3B, process 500 of FIG. 5A, process 600 of FIG. 6A, process 620 of FIG. 6B, process 640 of FIG. 6C, process 700 of FIG. 7, process 800 of FIG. 8A, and/or process 850 of FIG. 8B) is shown in FIG. 14. The computer system 1400 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage device 1430 in any suitable manner, as the aspects of the technology described herein are not limited to any particular techniques for writing or reading data. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing device 1400 may include a network input/output (I/O) interface 1440 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing device 1400 may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### Biological Samples

Any of the methods, systems, or other claimed elements may use or be used to analyze a biological sample from a subject. The biological sample may be any type of biological sample including, for example, a biological sample of a bodily fluid (e.g., blood, urine or cerebrospinal fluid), one or more cells (e.g., from a scraping or brushing such as a cheek swab or tracheal brushing), a piece of tissue (cheek tissue, muscle tissue, lung tissue, heart tissue, brain tissue, or skin tissue), or some or all of an organ (e.g., brain, lung, liver, bladder, kidney, pancreas, intestines, or muscle), or other types of biological samples (e.g., feces or hair).

In some embodiments, the biological sample is a sample of a tumor from a subject. In some embodiments, the biological sample is a sample of blood from a subject. In some embodiments, the biological sample is a sample of tissue from a subject.

A sample of a tumor, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells.

Examples of tumors include, but are not limited to, adenomas, fibromas, hemangiomas, lipomas, cervical dysplasia, metaplasia of the lung, leukoplakia, carcinoma, sarcoma, germ cell tumors, sex cord-stromal tumors, neuroendocrine tumors, gastrointestinal stromal tumors, and blastoma.

A sample of blood, in some embodiments, refers to a sample comprising cells, e.g., cells from a blood sample. In some embodiments, the sample of blood comprises non-cancerous cells. In some embodiments, the sample of blood comprises precancerous cells. In some embodiments, the sample of blood comprises cancerous cells. In some embodiments, the sample of blood comprises blood cells. In some embodiments, the sample of blood comprises red blood cells. In some embodiments, the sample of blood comprises white blood cells. In some embodiments, the sample of blood comprises platelets. Examples of cancerous blood cells include, but are not limited to, leukemia, lymphoma, and myeloma. In some embodiments, a sample of blood is collected to obtain the cell-free nucleic acid (e.g., cell-free DNA) in the blood.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

A sample of a tissue, in some embodiments, refers to a sample comprising cells from a tissue. In some embodiments, the sample of the tumor comprises non-cancerous cells from a tissue. In some embodiments, the sample of the tumor comprises precancerous cells from a tissue.

Methods of the present disclosure encompass a variety of tissue including organ tissue or non-organ tissue, including but not limited to, muscle tissue, brain tissue, lung tissue, liver tissue, epithelial tissue, connective tissue, and nervous tissue. In some embodiments, the tissue may be normal tissue, or it may be diseased tissue, or it may be tissue suspected of being diseased. In some embodiments, the tissue may be sectioned tissue or whole intact tissue. In some embodiments, the tissue may be animal tissue or human tissue. Animal tissue includes, but is not limited to, tissues obtained from rodents (e.g., rats or mice), primates (e.g., monkeys), dogs, cats, and farm animals.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, breast, and/or any type of tissue (e.g., muscle tissue, epithelial tissue, connective tissue, or nervous tissue).

Any of the biological samples described herein may be obtained from the subject using any known technique. Sec, for example, the following publications on collecting, processing, and storing biological samples, each of which are incorporated by reference herein in its entirety: Biospecimens and biorepositories: from afterthought to science by Vaught et al. (Cancer Epidemiol Biomarkers Prev. 2012 February; 21 (2): 253-5), and Biological sample collection, processing, storage and information management by Vaught and Henderson (IARC Sci Publ. 2011; (163): 23-42).

In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

In some embodiments, one or more than one cell (i.e., a cell biological sample) may be obtained from a subject using a scrape or brush method. The cell biological sample may be obtained from any area in or from the body of a subject including, for example, from one or more of the following areas: the cervix, esophagus, stomach, bronchus, or oral cavity. In some embodiments, one or more than one piece of tissue (e.g., a tissue biopsy) from a subject may be used. In certain embodiments, the tissue biopsy may comprise one or more than one (e.g., 2, 3, 4, 5, 6, 7, 8, 9, 10, or more than 10) biological samples from one or more tumors or tissues known or suspected of having cancerous cells.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one from to another such that the first form is no longer detected at the same level as before degradation.

In some embodiments, a biological sample (e.g., tissue sample) is fixed. As used herein, a “fixed” sample relates to a sample that has been treated with one or more agents or processes in order to prevent or reduce decay or degradation, such as autolysis or putrefaction, of the sample. Examples of fixative processes include but are not limited to heat fixation, immersion fixation, and perfusion. In some embodiments a fixed sample is treated with one or more fixative agents. Examples of fixative agents include but are not limited to cross-linking agents (e.g., aldehydes, such as formaldehyde, formalin, glutaraldehyde, etc.), precipitating agents (e.g., alcohols, such as ethanol, methanol, acetone, xylene, etc.), mercurials (e.g., B-5, Zenker's fixative, etc.), picrates, and Hepes-glutamic acid buffer-mediated organic solvent protection effect (HOPE) fixative. In some embodiments, a biological sample (e.g., tissue sample) is treated with a cross-linking agent. In some embodiments, the cross-linking agent comprises formalin. In some embodiments, a formalin-fixed biological sample is embedded in a solid substrate, for example paraffin wax. In some embodiments, the biological sample is a formalin-fixed paraffin-embedded (FFPE) sample. Methods of preparing FFPE samples are known, for example as described by Li et al. JCO Precis Oncol. 2018; 2: PO.17.00091.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 40° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris·Cl; 0.5 mM EDTA, pH 9.0)) and other coagulants, and Acids Citrate Dextronse (e.g., for blood specimens). In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −80° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

Methods of the present disclosure encompass obtaining one or more biological samples from a subject for analysis. In some embodiments, one biological sample is collected from a subject for analysis. In some embodiments, more than one (e.g., 2, 3, 4, 5, 6, 7, 8, 9, 10, 11, 12, 13, 14, 15, 16, 17, 18, 19, 20, or more) biological samples are collected from a subject for analysis. In some embodiments, one biological sample from a subject will be analyzed. In some embodiments, more than one (e.g., 2, 3, 4, 5, 6, 7, 8, 9, 10, 11, 12, 13, 14, 15, 16, 17, 18, 19, 20, or more) biological samples may be analyzed. If more than one biological sample from a subject is analyzed, the biological samples may be procured at the same time (e.g., more than one biological sample may be taken in the same procedure), or the biological samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure).

A second or subsequent biological sample may be taken or obtained from the same region (e.g., from the same tumor or area of tissue) or a different region (including, e.g., a different tumor). A second or subsequent biological sample may be taken or obtained from the subject after one or more treatments and may be taken from the same region or a different region. As a non-limiting example, the second or subsequent biological sample may be useful in determining whether the cancer in each biological sample has different characteristics (e.g., in the case of biological samples taken from two physically separate tumors in a subject) or whether the cancer has responded to one or more treatments (e.g., in the case of two or more biological samples from the same tumor or different tumors prior to and subsequent to a treatment). In some embodiments, each of the at least one biological sample is a bodily fluid sample, a cell sample, or a tissue biopsy sample.

In some embodiments, one or more biological specimens are combined (e.g., placed in the same container for preservation) before further processing. For example, a first sample of a first tumor obtained from a subject may be combined with a second sample of a second tumor from the subject, wherein the first and second tumors may or may not be the same tumor. In some embodiments, a first tumor and a second tumor are similar but not the same (e.g., two tumors in the brain of a subject). In some embodiments, a first biological sample and a second biological sample from a subject are sample of different types of tumors (e.g., a tumor in muscle tissue and brain tissue).

In some embodiments, a sample from which RNA and/or DNA is extracted (e.g., a sample of tumor, or a blood sample) is sufficiently large such that at least 2 μg (e.g., at least 2 μg, at least 2.5 μg, at least 3 μg, at least 3.5 μg or more) of DNA can be extracted from it. In some embodiments, the sample from which RNA and/or DNA is extracted can be peripheral blood mononuclear cells (PBMCs). In some embodiments, the sample from which RNA and/or DNA is extracted can be any type of cell suspension. In some embodiments, a sample from which RNA and/or DNA is extracted (e.g., a sample of tumor, or a blood sample) is sufficiently large such that at least 1.8 μg DNA can be extracted from it. In some embodiments, at least 50 mg (e.g., at least 1 mg, at least 2 mg, at least 3 mg, at least 4 mg, at least 5 mg, at least 10 mg, at least 12 mg, at least 15 mg, at least 18 mg, at least 20 mg, at least 22 mg, at least 25 mg, at least 30 mg, at least 35 mg, at least 40 mg, at least 45 mg, or at least 50 mg) of tissue sample is collected from which RNA and/or DNA is extracted. In some embodiments, at least 20 mg of tissue sample is collected from which RNA and/or DNA is extracted. In some embodiments, at least 30 mg of tissue sample is collected. In some embodiments, at least 10-50 mg (e.g., 10-50 mg, 10-15 mg, 10-30 mg, 10-40 mg, 20-30 mg, 20-40 mg, 20-50 mg, or 30-50 mg) of tissue sample is collected from which RNA and/or DNA is extracted. In some embodiments, at least 30 mg of tissue sample is collected. In some embodiments, at least 20-30 mg of tissue sample is collected from which RNA and/or DNA is extracted. In some embodiments, a sample from which RNA and/or DNA is extracted (e.g., a sample of tumor, or a blood sample) is sufficiently large such that at least 0.2 μg (e.g., at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of DNA can be extracted from it. In some embodiments, a sample from which RNA and/or DNA is extracted (e.g., a sample of tumor, or a blood sample) is sufficiently large such that at least 0.1 μg (e.g., at least 100 ng, at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of DNA can be extracted from it.

### Subjects

Aspects of this disclosure relate to a tumor sample that has been obtained from one or more subjects. In some embodiments, a subject is a mammal (e.g., a human, a mouse, a cat, a dog, a horse, a hamster, a cow, a pig, or other domesticated animal, a farm animal (e.g., livestock), a sport animal, a laboratory animal, a pet, and a primate). In some embodiments, a subject is a human. In some embodiments, a subject is an adult human (e.g., of 18 years of age or older). In some embodiments, a subject is a child (e.g., less than 18 years of age).

### Sequencing Data

Aspects of the disclosure relate to predicting whether a subject will respond to a therapy (e.g., an immune checkpoint inhibitor therapy) based on sequencing data and/or RNA expression data obtained from a biological sample (e.g., a tumor sample and/or a blood sample).

The RNA expression data used in methods described herein typically is derived from sequencing data obtained from the biological sample.

The sequencing data may be obtained from the biological sample using any suitable sequencing technique and/or apparatus (e.g., sequencing platform 106 shown in FIG. 1A and/or sequencing platform 260 shown in FIG. 2). In some embodiments, the sequencing apparatus used to sequence the biological sample may be selected from any suitable sequencing apparatus known in the art including, but not limited to, Illumina™, SOLid™, Ion Torrent™, PacBio™, a nanopore-based sequencing apparatus, a Sanger sequencing apparatus, or a 454™ sequencing apparatus. In some embodiments, sequencing apparatus used to sequence the biological sample is an Illumina sequencing (e.g., NovaSeq™, NextSeq™, HiSeq™, MiSeq™, or MiniSeq™) apparatus.

After the sequencing data is obtained, it is processed in order to obtain the RNA expression data. RNA expression data may be acquired using any method known in the art including, but not limited to whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, RNA expression data may be obtained using a microarray assay.

In some embodiments, the sequencing data is processed to produce RNA expression data. In some embodiments, RNA sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Páll Melsted and Lior Pachter, Near-optimal probabilistic RNA-seq quantification, Nature Biotechnology 34, 525-527 (2016), doi: 10.1038/nbt.3519, which is incorporated by reference in its entirety herein.

In some embodiments, microarray expression data is processed using a bioinformatics R package, such as “affy” or “limma,” in order to produce expression data. The “affy” software is described in Bioinformatics. 2004 Feb. 12; 20 (3): 307-15. doi: 10.1093/bioinformatics/btg405. “affy--analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law C W, Shi W, Smyth G K “limma powers differential expression analyses for RNA-sequencing and microarray studies.” Nucleic Acids Res. 2015 Apr. 20; 43 (7):e47. 20. doi.org/10.1093/nar/gkv007PMID: 25605792, PMCID: PMC4402510, which is incorporated by reference herein its entirety.

In some embodiments, sequencing data and/or expression data comprises more than 5 kilobases (kb). In some embodiments, the size of the obtained RNA data is at least 10 kb. In some embodiments, the size of the obtained RNA sequencing data is at least 100 kb. In some embodiments, the size of the obtained RNA sequencing data is at least 500 kb. In some embodiments, the size of the obtained RNA sequencing data is at least 1 megabase (Mb). In some embodiments, the size of the obtained RNA sequencing data is at least 10 Mb. In some embodiments, the size of the obtained RNA sequencing data is at least 100 Mb. In some embodiments, the size of the obtained RNA sequencing data is at least 500 Mb. In some embodiments, the size of the obtained RNA sequencing data is at least 1 gigabase (Gb). In some embodiments, the size of the obtained RNA sequencing data is at least 10 Gb. In some embodiments, the size of the obtained RNA sequencing data is at least 100 Gb. In some embodiments, the size of the obtained RNA sequencing data is at least 500 Gb.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the expression data comprises next-generation sequencing (NGS) data. In some embodiments, the expression data comprises microarray data.

In some embodiments, the sequencing data comprises cellular indexing of transcriptomes and epitopes by sequencing (CITE-seq) data. In some embodiments, the sequencing data comprises DNA methylation data.

Expression data (e.g., indicating expression levels) for a plurality of genes may be used for any of the methods or compositions described herein. The number of genes which may be examined may be up to and inclusive of all the genes of the subject. In some embodiments, expression levels may be determined for all of the genes of a subject. As a non-limiting example, In some embodiments, expression levels may be obtained for at least 25 genes, at least 50 genes, at least 75 genes, at least 100 genes, at least 150 genes, at least 200 genes, at least 250 genes, at least 500 genes, at least 1,000 genes, at least 1,500 genes, at least 2,000 genes, at least 2,500 genes, at least 3,000 genes, at least 3,500 genes, at least 4,000 genes, at least 4,500 genes, at least 5,000 genes, at least 6000 genes, at least 7,000 genes, at least 8,000 genes, at least 9,000 genes, at least 10,000 genes, at least 15,000 genes, at least 20,000 genes, or at least any other suitable number of genes, as aspects of the technology described herein are not limited in this respect. In some embodiments, expression levels may be obtained for at most 25 genes, at most 50 genes, at most 75 genes, at most 100 genes, at most 150 genes, at most 200 genes, at most 250 genes, at most 500 genes, at most 1,000 genes, at most 1,500 genes, at most 2,000 genes, at most 2,500 genes, at most 3,000 genes, at most 3,500 genes, at most 4,000 genes, at most 4,500 genes, at most 5,000 genes, at most 6000 genes, at most 7,000 genes, at most 8,000 genes, at most 9,000 genes, at most 10,000 genes, at most 15,000 genes, at most 20,000 genes, or at most any other suitable number of genes, as aspects of the technology described herein are not limited in this respect. It should be appreciated that any of the above-listed upper bounds may be coupled with any of the above-listed lower bounds. In some embodiments, As another set of non-limiting examples, the expression data may include, for each set of genes listed in Table 1, expression data for at least some (e.g., all) of the genes included in the particular set of genes.

In some embodiments, RNA expression data is obtained by accessing the RNA expression data from at least one computer storage medium on which the RNA expression data is stored. Additionally or alternatively, in some embodiments, RNA expression data may be received from one or more sources via a communication network of any suitable type. For example, in some embodiment, the RNA expression data may be received from a server (e.g., a SFTP server, or Illumina BaseSpace).

The RNA expression data obtained may be in any suitable format, as aspects of the technology described herein are not limited in this respect. For example, in some embodiments, the RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

In some embodiments, sequencing data is processed to obtain RNA expression data from the sequencing data. For example, the sequencing data may be processed using any suitable computing device or devices, as aspects of the technology described herein are not limited in this respect. For example, the processing may be performed by a computing device part of a sequencing apparatus. In other embodiments, the processing may be performed by one or more computing devices external to the sequencing apparatus.

In some embodiments, processing the sequencing data to obtain RNA expression data from the sequencing data includes expressing the sequencing data in TPM units. This may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM conversion may be performed using a software package, such as, for example, the germa package. Aspects of the germa package are described in Wu J, Gentry RIwcfJMJ (2021). “germa: Background Adjustment Using Sequence Information. R package version 2.66.0,” which is incorporated by reference in its entirety herein. In some embodiments, RNA expression level in TPM units for a particular gene may be calculated according to the following formula:

$$A \cdot \frac{1}{\sum ( A )} \cdot {10}^{6} where A = \frac{total reads mapped to gene \cdot {10}^{3}}{gene length in bp}$$

Next, in some embodiments, the RNA expression levels in TPM units may be log transformed.

In some embodiments, the RNA expression levels may not be expressed in TPM units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

In some embodiments, the RNA expression data is obtained by processing sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by processing the sequence data can include information included in a FASTA file, a description and/or quality scores included in a FASTQ file, an aligned position included in a BAM file, and/or any other suitable information obtained from any suitable file.

In some embodiments, enrichment scores for genes in one or more sets of genes (e.g., gene groups) are determined. For example, an enrichment score may be determined for at least some genes listed for one or more of the gene groups in Table 8. In some embodiments, an enrichment score is generated using a gene set enrichment analysis (GSEA) technique, using RNA expression levels of at least some genes in a set of genes. In some embodiments, using a GSEA technique comprises using single-sample GSEA. Aspects of single sample GSEA (ssGSEA) are described in Barbie et al. Nature. 2009 Nov. 5; 462 (7269): 108-112, the entire contents of which are incorporated by reference herein. In some embodiments, ssGSEA is performed according to the following formula:

$$ssGSEA score = \frac{{\sum }_{i}^{N} {r}_{i}^{1.25}}{{\sum }_{i}^{N} {r}_{i}^{0.25}} - \frac{( M - N + 1 )}{2}$$

where rᵢ represents the rank of the ith gene in expression matrix, where N represents the number of genes in the gene set, and where M represents total number of genes in expression matrix. Additional, suitable techniques of performing GSEA are known in the art and are contemplated for use in the methods described herein without limitation.

### Flow Cytometry

Aspects of the disclosure relate to predicting whether a subject will respond to a therapy based on cytometry data obtained from a blood sample. In some embodiments, the cytometry data is flow cytometry data.

In some embodiments, a flow cytometry platform may be used to perform flow cytometry investigation of a fluid sample. The fluid sample may include target particles with particular particle attributes. The flow cytometry investigation of the fluid sample may provide a flow cytometry result for the fluid sample.

In some embodiments, the fluid sample may be exposed to a stain or dye that provides response radiation when exposed to investigation excitation radiation that may be measured by the radiation detection system of the flow cytometry platform. In some embodiments, a multiplicity of photodetectors are included in the flow cytometry platform. When a particle passes through the laser beam, time correlated pulses on forward scatter (FSC) and side scatter (SSC) detectors, and possibly also fluorescent emission detectors will occur. This is an “event,” and for each event the magnitude of the detector output for each detector, FSC, SSC and fluorescence detectors is stored. The data obtained comprise the signals measured for each of the light scatter parameters and the fluorescence emissions.

Flow cytometry platforms may further comprise components for storing the detector outputs and analyzing the data. For example, data storage and analysis may be carried out using a computer connected to the detection electronics. For example, the data can be stored logically in tabular form, where each row corresponds to data for one particle (or one event), and the columns correspond to each of the measured parameters. The use of standard file formats, such as an “FCS” file format, for storing data from a flow cytometer facilitates analyzing data using separate programs and/or machines. In some embodiments, the data may be displayed in 2-dimensional (2D) plots for ease of visualization, but other methods may be used to visualize multidimensional data.

In some embodiments, the parameters measured using a flow cytometer may include FSC, which refers to the excitation light that is scattered by the particle along a generally forward direction, SSC, which refers to the excitation light that is scattered by the particle in a generally sideways direction, and the light emitted from fluorescent molecules in one or more channels (frequency bands) of the spectrum, referred to as FL1, FL2, etc., or by the name of the fluorescent dye that emits primarily in that channel.

Both flow and scanning cytometers are commercially available from, for example, BD Biosciences (San Jose, Calif.). Flow cytometry is described in, for example, Landy et al. (eds.), Clinical Flow Cytometry, Annals of the New York Academy of Sciences Volume 677 (1993); Bauer et al. (eds.), Clinical Flow Cytometry: Principles and Applications, Williams & Wilkins (1993); Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); and Practical Shapiro, Flow Cytometry, 4th ed., Wiley-Liss (2003); all incorporated herein by reference. Fluorescence imaging microscopy is described in, for example, Pawley (ed.), Handbook of Biological Confocal Microscopy, 2nd Edition, Plenum Press (1989), incorporated herein by reference.

### Mass Cytometry

Aspects of the disclosure relate to predicting whether a subject will respond to a therapy based on cytometry data obtained from a blood sample. In some embodiments, the cytometry data is mass cytometry data.

In some embodiments, a mass cytometry platform may be used to perform mass cytometry investigation of a fluid sample. The fluid sample may include target particles with particular particle attributes. The mass cytometry investigation of the fluid sample may provide a mass cytometry result for the fluid sample.

In some embodiments, the fluid sample may be exposed to target-specific antibodies labeled with metal isotopes. In some embodiments, elemental mass spectrometry (e.g., inductively coupled plasma mass spectrometry (ICP-MS) and time of flight mass spectrometry (TOF-MS)) is used to detect the conjugated antibodies. For example, elemental mass spectrometry can discriminate isotopes of different atomic weights and measure electrical signals for isotopes associated with each particle or cell. Data obtained for a single cell or particle is considered an “event.”

Mass cytometry platforms may further comprise components for storing the detector outputs and analyzing the data. For example, data storage and analysis may be carried out using a computer connected to the detection elements. The use of standard file formats, such as an “FCS” file format, for storing data from a mass cytometry platform facilitates analyzing data using separate programs and/or machines.

Mass cytometry platforms are commercially available from, for example, Fluidigm (San Francisco, CA). Mass cytometry is described in, for example, Bendall et al., A deep profiler's guide to cytometry, Trends in Immunology, 33 (7), 323-332 (2012) and Spitzer et al., Mass Cytometry: Single Cells, Many Features, Cell, 165 (4), 780-791 (2016), both of which are incorporated by reference herein in their entirety.

### Spectral Cytometry

Aspects of the disclosure relate to predicting whether a subject will respond to a therapy based on cytometry data obtained from a blood sample. In some embodiments, the cytometry data is spectral cytometry data.

In some embodiments, a spectral cytometry platform may be used to perform spectral cytometry investigation of a fluid sample. The fluid sample may include target particles with particular particle attributes. The spectral cytometry investigation of the fluid sample may provide a spectral cytometry result for the fluid sample.

In some embodiments, the fluid sample may be exposed to a stain or dye that provides response radiation when exposed to investigation excitation radiation that may be measured by the radiation detection system of the spectral cytometry platform. In some embodiments, a multiplicity of photodetectors are included in the spectral cytometry platform. When a particle passes through the laser beam, time correlated pulses on forward scatter (FSC) and side scatter (SSC) detectors, and possibly also fluorescent emission detectors will occur. This is an “event,” and for each event the magnitude of the detector output for each detector, FSC, SSC and fluorescence detectors is stored. The data obtained comprise the signals measured for each of the light scatter parameters and the fluorescence emissions.

Compared to conventional spectral cytometry, spectral cytometry may utilize a full spectrum of light to distinguish one fluorophore from another. For example, spectral cytometry may utilize multiple (e.g., all) detectors for all fluorophores.

Spectral cytometry platforms may further comprise components for storing the detector outputs and analyzing the data. For example, data storage and analysis may be carried out using a computer connected to the detection electronics. For example, the data can be stored logically in tabular form, where each row corresponds to data for one particle (or one event), and the columns correspond to each of the measured parameters. The use of standard file formats, such as an “FCS” file format, for storing data from a spectral cytometer facilitates analyzing data using separate programs and/or machines. In some embodiments, the data may be displayed in 2-dimensional (2D) plots for ease of visualization, but other methods may be used to visualize multidimensional data.

### Therapies

Aspects of the disclosure relate to methods of identifying or selecting a therapy agent (e.g., an immune checkpoint inhibitor (ICI)) for a subject based on RNA expression data from a tumor sample and cell population data from a blood sample. The disclosure is based, in part, on the recognition that subjects may have an increased likelihood of responding to certain therapies based on one or more characteristics of the tumor sample and one or more characteristics of the blood sample.

In some embodiments, the therapeutic agents are immune checkpoint inhibitors. Examples of immune checkpoint inhibitors include pembrolizumab, ipilimumab, nivolumab, cemiplimab, dostarlimab, atezolizumab, durvalumab, and avelumab.

In some embodiments, methods described by the disclosure further comprise a step of administering one or more therapeutic agents to the subject based upon a prediction of therapeutic response. In some embodiments, a subject is administered one or more (e.g., 1, 2, 3, 4, 5, or more) immune checkpoint inhibitors.

Aspects of the disclosure relate to methods of treating a subject having (or suspected or at risk of having) cancer based upon a prediction of therapeutic response. In some embodiments, the methods comprise administering one or more (e.g., 1, 2, 3, 4, 5, or more) therapeutic agents to the subject.

The subject to be treated by the methods described herein may be a human subject having, suspected of having, or at risk for a cancer. Examples of a cancer include, but are not limited to, melanoma, lung cancer, brain cancer, breast cancer, colorectal cancer, pancreatic cancer, liver cancer, skin cancer, kidney cancer, bladder cancer, ovarian cancer, cervical cancer, or prostate cancer. At the time of diagnosis, the cancer may be cancer of unknown primary.

A subject having a cancer may be identified by routine medical examination, e.g., laboratory tests, biopsy, PET scans, CT scans, or ultrasounds. A subject suspected of having a cancer might show one or more symptoms of the disorder, e.g., unexplained weight loss, fever, fatigue, cough, pain, skin changes, unusual bleeding or discharge, and/or thickening or lumps in parts of the body. A subject at risk for a cancer may be a subject having one or more of the risk factors for that disorder. For example, risk factors associated with cancer include, but are not limited to, (a) viral infection (e.g., herpes virus infection), (b) age, (c) family history, (d) heavy alcohol consumption, (e) obesity, and (f) tobacco use.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor formation, tumor growth, molecular category identified for the cancer using the techniques described herein) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays. The dosing regimen (including the therapeutic used) may vary over time.

When the anti-cancer therapeutic agent is not an antibody, it may be administered at the rate of about 0.1 to 300 mg/kg of the weight of the subject divided into one to three doses, or as disclosed herein. In some embodiments, for an adult subject of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known in the art).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the subject's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of the cancer, or the predisposition toward a cancer.

Alleviating a cancer includes delaying the development or progression of the disease or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given period and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. Alternatively, or in addition to the clinical techniques known in the art, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

In some embodiments, the anti-cancer therapeutic agent described herein is administered to a subject in need of the treatment at an amount sufficient to reduce cancer (e.g., tumor) growth by at least 10% (e.g., 20%, 30%, 40%, 50%, 60%, 70%, 80%, 90% or greater). In some embodiments, the anti-cancer therapeutic agent described herein is administered to a subject in need of the treatment at an amount sufficient to reduce cancer cell number or tumor size by at least 10% (e.g., 20%, 30%, 40%, 50%, 60%, 70%, 80%, 90% or more). In other embodiments, the anti-cancer therapeutic agent is administered in an amount effective in altering cancer type. Alternatively, the anti-cancer therapeutic agent is administered in an amount effective in reducing tumor formation or metastasis.

Conventional methods, known to those of ordinary skill in the art of medicine, may be used to administer the anti-cancer therapeutic agent to the subject, depending upon the type of disease to be treated or the site of the disease. The anti-cancer therapeutic agent can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, an anti-cancer therapeutic agent may be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (e.g., glycerol, propylene glycol, liquid polyethylene glycol, and the like). For intravenous injection, water soluble anti-cancer therapeutic agents can be administered by the drip method, whereby a pharmaceutical formulation containing the antibody and a physiologically acceptable excipients is infused. Physiologically acceptable excipients may include, for example, 5% dextrose, 0.9% saline, Ringer's solution, and/or other suitable excipients. Intramuscular preparations, e.g., a sterile formulation of a suitable soluble salt form of the anti-cancer therapeutic agent, can be dissolved and administered in a pharmaceutical excipient such as Water-for-Injection, 0.9% saline, and/or 5% glucose solution.

In one embodiment, an anti-cancer therapeutic agent is administered via site-specific or targeted local delivery techniques. Examples of site-specific or targeted local delivery techniques include various implantable depot sources of the agent or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568, the contents of each of which are incorporated by reference herein for this purpose.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. The contents of each of the foregoing are incorporated by reference herein for this purpose.

Therapeutic compositions containing a polynucleotide may be administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

Therapeutic polynucleotides and polypeptides can be delivered using gene delivery vehicles. The gene delivery vehicle can be of viral or non-viral origin (e.g., Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). The contents of each of the foregoing are incorporated by reference herein for this purpose. Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed. The contents of each of the foregoing are incorporated by reference herein for this purpose.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP U.S. Pat. No. 524,968. Additional approaches are described in Philip, Mol. Cell. Biol. (1994) 14:2411, and in Woffendin, Proc. Natl. Acad. Sci. (1994) 91:1581. The contents of each of the foregoing are incorporated by reference herein for this purpose.

It is also apparent that an expression vector can be used to direct expression of any of the protein-based anti-cancer therapeutic agents (e.g., anti-cancer antibody). For example, peptide inhibitors that are capable of blocking (from partial to complete blocking) a cancer-causing biological activity are known in the art.

In some embodiments, more than one anti-cancer therapeutic agent, such as an antibody and a small molecule inhibitory compound, may be administered to a subject in need of the treatment. The agents may be of the same type or different types from each other. At least one, at least two, at least three, at least four, or at least five different agents may be co-administered. Generally anti-cancer agents for administration have complementary activities that do not adversely affect each other. Anti-cancer therapeutic agents may also be used in conjunction with other agents that serve to enhance and/or complement the effectiveness of the agents.

Treatment efficacy can be assessed by methods well-known in the art, e.g., monitoring tumor growth or formation in a subject subjected to the treatment. Alternatively, or in addition to, treatment efficacy can be assessed by monitoring tumor type over the course of treatment (e.g., before, during, and after treatment).

A subject having cancer may be treated using any combination of anti-cancer therapeutic agents or one or more anti-cancer therapeutic agents and one or more additional therapies (e.g., surgery and/or radiotherapy). The term combination therapy, as used herein, embraces administration of more than one treatment (e.g., an antibody and a small molecule or an antibody and radiotherapy) in a sequential manner, that is, wherein each therapeutic agent is administered at a different time, as well as administration of these therapeutic agents, or at least two of the agents or therapies, in a substantially simultaneous manner.

Sequential or substantially simultaneous administration of each agent or therapy can be affected by any appropriate route including, but not limited to, oral routes, intravenous routes, intramuscular, subcutaneous routes, and direct absorption through mucous membrane tissues. The agents or therapies can be administered by the same route or by different routes. For example, a first agent (e.g., a small molecule) can be administered orally, and a second agent (e.g., an antibody) can be administered intravenously.

As used herein, the term “sequential” means, unless otherwise specified, characterized by a regular sequence or order, e.g., if a dosage regimen includes the administration of an antibody and a small molecule, a sequential dosage regimen could include administration of the antibody before, simultaneously, substantially simultaneously, or after administration of the small molecule, but both agents will be administered in a regular sequence or order. The term “separate” means, unless otherwise specified, to keep apart one from the other. The term “simultaneously” means, unless otherwise specified, happening or done at the same time, i.e., the agents are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents or therapies described herein.

Combination therapy can also embrace the administration of the anti-cancer therapeutic agent (e.g., an antibody) in further combination with other biologically active ingredients (e.g., a vitamin) and non-drug therapies (e.g., surgery or radiotherapy).

It should be appreciated that any combination of anti-cancer therapeutic agents may be used in any sequence for treating a cancer. The combinations described herein may be selected on the basis of a number of factors, which include but are not limited to reducing tumor formation or tumor growth, and/or alleviating at least one symptom associated with the cancer, or the effectiveness for mitigating the side effects of another agent of the combination. For example, a combined therapy as provided herein may reduce any of the side effects associated with each individual members of the combination, for example, a side effect associated with an administered anti-cancer agent.

In some embodiments, an anti-cancer therapeutic agent is an antibody, an immunotherapy, a radiation therapy, a surgical therapy, and/or a chemotherapy.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor, a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, Carboplatin or Cisplatin, Docetaxel, Gemcitabine, Nab-Paclitaxel, Paclitaxel, Pemetrexed, and Vinorelbine.

Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurca, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately.” “substantially.” and “about” may include the target value.

1. A method for predicting whether a subject will respond to an immune checkpoint inhibitor (ICI) therapy based on RNA expression data and cytometry data obtained for the subject, the method comprising:
using at least one computer hardware processor to perform:
obtaining the RNA expression data, the RNA expression data having been previously obtained from a tumor sample from the subject;
selecting, from among multiple molecular-functional (MF) profile types and using the RNA expression data, an MF profile type for the tumor sample;
obtaining the cytometry data, the cytometry data having been previously obtained from a blood sample from the subject;
determining, using the cytometry data, a G2 score for the blood sample, wherein the G2 score is indicative a likelihood that the blood sample is of a Primed (G2) immunoprofile type of multiple immunoprofile types; and
predicting, using a statistical model and based on the selected MF profile type and the G2 score, whether the subject will respond to the ICI therapy.

2. The method of claim 1, further comprising:
after predicting that the subject will respond to the ICI therapy, recommending the ICI therapy for the subject or selecting the subject for treatment with the ICI therapy.

3. The method of claim 2, further comprising:
administering the ICI therapy to the subject.

4. The method of claim 1, wherein the ICI therapy comprises anti-PD-1 antibodies, anti-CTLA4 antibodies, and/or anti-PD-L1 antibodies.

5. The method of claim 1, wherein predicting whether the subject will respond to the ICI therapy comprises processing the selected MF profile type and the G2 score with the statistical model.

6. The method of claim 1, wherein the statistical model is a generalized linear model.

7. The method of claim 1, further comprising:
determining, based on the RNA expression data, an expression of PD-L1 in the tumor sample,
wherein determining whether the subject will respond to the ICI therapy comprises processing the selected MF profile type, the G2 score, and the expression of PD-L1 in the tumor sample using the statistical model.

8. The method of claim 1, wherein selecting the MF profile type for the tumor sample comprises:
determining, using the RNA expression data, an MF profile for the tumor sample at least in part by determining a gene group expression level for each gene group in a set of gene groups; and
selecting, using the MF profile, the MF profile type for the tumor sample.

9. The method of claim 1, further comprising:
encoding the MF profile type selected for the tumor sample to obtain an encoded MF profile type, the encoding comprising:
assigning a first value to the MF profile type when the MF profile type is a first MF profile type or a second MF profile type of the multiple MF profile types; and
assigning a second value to the MF profile type when the MF profile type is a third MF profile type or a fourth MF profile type of the multiple MF profile types, wherein the second value is different from the first value; and
determining whether the subject will respond to the ICI therapy based on the encoded MF profile type and the G2 score.

10. The method of claim 9, wherein:
the first MF profile type is associated with inflamed and vascularized tumor samples and/or inflamed and fibroblast-enriched tumor samples,
the second MF profile type is associated with inflamed and non-vascularized tumor samples and/or inflamed and non-fibroblast-enriched tumor samples,
the third MF profile type is associated with non-inflamed and vascularized tumor samples and/or non-inflamed and fibroblast-enriched tumor samples, and
the fourth MF profile type is associated with non-inflamed and non-vascularized tumor samples and/or non-inflamed and non-fibroblast-enriched tumor samples.

11. The method of claim 1, wherein determining the G2 score using the cytometry data comprises:
processing the cytometry data to determine cytometry-based cell composition percentages for a plurality of types of cells in the blood sample; and
determining the G2 score using the cytometry-based cell composition percentages.

12. The method of claim 11, wherein determining the G2 score using the cytometry-based cell composition percentages comprises processing the cytometry-based cell composition percentages using a G2 score statistical model trained to predict the G2 score.

13. The method of claim 11, wherein processing the cytometry data to determine the cytometry-based cell composition percentages comprises:
processing the cytometry data using one or more machine learning models to identify the types of the cells in the blood sample; and
determining the cytometry-based cell composition percentages based on the identified types of the cells in the blood sample.

14. The method of claim 1, wherein the RNA expression data for the tumor sample is first RNA expression data, and wherein the method further comprises:
obtaining second RNA expression data, the second RNA expression data having been previously obtained from the blood sample from the subject, and
wherein determining the G2 score comprises determining the G2 score using the cytometry data or the second RNA expression data.

15. The method of claim 14, wherein determining the G2 score using the second RNA expression data comprises:
processing the second RNA expression data to determine RNA-based cell composition percentages for types of cells in the blood sample; and
determining the G2 score using the RNA-based cell composition percentages.

16. The method of claim 15, wherein determining the G2 score using the RNA-based cell composition percentages comprises:
processing the RNA-based cell composition percentages using a G2 score statistical model trained to predict the G2 score.

17. The method of claim 1, wherein the multiple immunoprofile types comprise:
a Naive (G1) immunoprofile type, the Primed (G2) immunoprofile type, a Progressive (G3) immunoprofile type, a Chronic (G4) immunoprofile type, and a Suppressive (G5) immunoprofile type.

18. The method of claim 1, wherein the subject has, is suspected of having, or is at risk of having carcinoma.

19. At least one non-transitory, computer-readable storage medium storing processor-executable instructions that, when executed by at least one computer hardware processor, cause the at least one computer hardware processor to perform a method for predicting whether a subject will respond to an immune checkpoint inhibitor (ICI) therapy based on RNA expression data and cytometry data obtained for the subject, the method comprising:
obtaining the RNA expression data, the RNA expression data having been previously obtained from a tumor sample from the subject;
selecting, from among multiple molecular-functional (MF) profile types and using the RNA expression data, an MF profile type for the tumor sample;
obtaining the cytometry data, the cytometry data having been previously obtained from a blood sample from the subject;
determining, using the cytometry data, a G2 score for the blood sample, wherein the G2 score is indicative a likelihood that the blood sample is of a Primed (G2) immunoprofile type of multiple immunoprofile types; and
predicting, using a statistical model and based on the selected MF profile type and the G2 score, whether the subject will respond to the ICI therapy.

20. A system, comprising:
at least one computer hardware processor; and
at least one non-transitory computer-readable storage medium storing processor-executable instructions that, when executed by the at least one computer hardware processor, cause the at least one computer hardware processor to perform method for predicting whether a subject will respond to an immune checkpoint inhibitor (ICI) therapy based on RNA expression data and cytometry data obtained for the subject, the method comprising:
obtaining the RNA expression data, the RNA expression data having been previously obtained from a tumor sample from the subject;
selecting, from among multiple molecular-functional (MF) profile types and using the RNA expression data, an MF profile type for the tumor sample;
obtaining the cytometry data, the cytometry data having been previously obtained from a blood sample from the subject;
determining, using the cytometry data, a G2 score for the blood sample, wherein the G2 score is indicative a likelihood that the blood sample is of a Primed (G2) immunoprofile type of multiple immunoprofile types; and
predicting, using a statistical model and based on the selected MF profile type and the G2 score, whether the subject will respond to the ICI therapy.
